(19) United States
(12) Patent Application Publication
(10) Pub. No.: US 2016/0159914 A1
(43) Pub. Date: Jun. 9, 2016

(54) IL-17 RECEPTOR A IS REQUIRED FOR IL-17C BIOLOGY

(71) Applicant: AMGEN INC., Thousand Oaks, CA (US)

(72) Inventors: Alison L. Budelsky, Seattle, WA (US); Jennifer E. Towne, Seattle, WA (US); Yu Zhang, Shoreline, WA (US); Lori Siegel, Kingston, WA (US); Antony Symons, Seattle, WA (US)

(21) Appl. No.: 14/886,565

(22) Filed: Oct. 19, 2015

Related U.S. Application Data
(63) Continuation of application No. 14/234,064, filed on Apr. 21, 2014.
(60) Provisional application No. 61/620,888, filed on Apr. 5, 2012.
(60) Provisional application No. 61/510,930, filed on Jul. 22, 2011.

(51) Int. Cl. C07K 16/28

(52) U.S. Cl. CPC C07K 16/2866; C07K 2317/76; C07K 2317/24; C07K 2317/20; A61K 2039/505

(57) ABSTRACT

The present invention relates to Interleukin-17 ligand and receptor family members and the discovery that IL-17 receptor A and IL-17 receptor E form a heteromeric receptor complex that is biologically active, and that IL-17C activity requires the IL-17RA-IL-17RE heteromeric receptor complex. Antagonists of the IL-17RA-IL-17RE heteromeric receptor complex are disclosed, as well as various methods of use.

### CROSS REFERENCE TO RELATED APPLICATIONS

This application claims the benefit under 35 U.S.C. 119(e) of U.S. patent application No. 61/620,888, filed Apr. 5, 2012 and U.S. patent application No. 61/510,930, filed Jul. 22, 2011, which are incorporated herein by reference.

### FIELD OF THE INVENTION

The present invention relates to Interleukin-17 ligand and receptor family members and the discovery that IL-17 Receptor A (IL-17RA) is required in an IL-17RA-IL-17RE heteromeric receptor complex for the biological activity of IL-17C. Antagonists of the IL-17RA-IL-17RE heteromeric receptor complex that inhibit the biological activity of IL-17C and methods of use are described. Monoclonal antibodies that specifically bind IL-17RA that inhibit IL-17C activation of the IL-17RA-IL-17RE heteromeric receptor complex are encompassed by the invention.

### BACKGROUND OF THE INVENTION

The IL-17 family is composed of six cytokines and five receptors. The ligand-receptor paring is not completely resolved for all members (Ely, et al., 2009, Nat. Immunol. 10:121245-1251). IL-17A is expressed by a unique lineage of CD4 positive T cells (T_{H}-17) that develop in response to IL-23, in particular under conditions in which T_{H}1 and T_{H}2 development are suppressed (Hanington, et al., 2005, Nat. Immunol. 6:1123-1132; Langrish, et al., 2005, J. Exp. Afed. 201:233-240; Park, et al., 2005, Nat. Immunol. 6:1133-1141; Aggarwal, et al., 2003, J. Biol. Chem. 278:1910-1914). In addition to T_{H}-17 cells, many innate immune cells such as gamma delta (γδ) T cells, natural killer (NK) T cells, neutrophils, and lymphoid-tissue inducer (LTi) cells produce IL-17A and IL-17F (reviewed in Cua and Tato, 2010). Evidence shows that IL-17A is implicated in rheumatoid arthritis, psoriasis, inflammatory bowel disease, multiple sclerosis, and asthma, and plays a role in host defense (Matusevicius, et al., 1999, Multiple Sclerosis. 5:101-104; Molet, et al., 2001, J. Allergy Clin. Immunol. 108:430-438; Lock, et al., 2002, Nat. Med. 8(5):500-508; Barczyk, et al., 2003, Resp. Med. 97:726-733; Kolls, et al., 2004, Immunity 21:467-476; Koenders, et al., 2005, A. J. Pathol 167:141-149; Lubberts, et al., 2005, J. Immunol. 175:3360-3368; Koenders, et al., 2005, Arthritis Rheum. 52:3239-3247; Nakae, et al., 2003, J. Immunol. 171:6173-6177; Wang, et al., 2007, J. Exp. Med. 204: 1837-1847; Hata, et al., 2008, Inflamm Bowel Dis. 14:1175-1184; Al-Ramli et al., 2009, Journal Allergy Clinical Immunol. 123:1185-1187; Brand, 2009, Gut. 58:1152-1167; Durelli, et al., 2009, Ann. Neural. 65:499-509; Rovedatti, et al., 2009, Gut. 58:1629-1636; Axtell, et al., 2010, Nat. Med. 16:406-413; Babaloo, et al., 2010, Iran. J. Immunol., 7:202-209.)

IL-17C has been reported to bind IL-17RE and to activate NK-κB. Ectopic expression of IL-17C by CD4+ T-cells exacerbates collagen-induced arthritis, and intranasal administration of adnoviruses expressing IL-17C triggers comparable responses by neutrophils as does IL-17A and IL-17F, suggesting these cytokines mediate common biological effects (Hurst, et al., 2002, J. Immunol. 169:443-453; Yamaguchi, et al., 2007, J. Immunol. 179:7128-7136). Levels of IL-17A, IL-17C, and IL-17F mRNA are elevated in lesional skin from psoriasis patients (Johansen C, et al., Br J Dermatol 2009; 160(2):319-24).

IL-17 receptors form a family of related Type I transmembrane proteins. Five different members of this family have been identified (IL-17RA through IL-17RE), several of which also display alternative splicing including soluble forms that may act as decoy receptors. IL-17RA is a necessary receptor for multiple IL-17 family cytokines including IL-17A, IL-17F, IL-17A/F heterodimer and IL-25. IL-17RA can multimerize independent of ligand and has been shown to form a biologically active heteromeric receptor complex with IL-17RC (Toy et al., J Immunol. 177:36; 2007). In addition, IL-17RA forms a biologically active heteromeric receptor with IL-17RB (Rickel, et al., J Immunol, 181, 42-94310 (2008). For a review of the IL-17 ligands and receptors see Gaffen, Nat Rev Immun, vol. 9, pages 556-567, August 2009.

The present invention shows that IL-17RA forms a biologically functional heteromeric receptor complex with IL-17 Receptor E (IL-17RE) and that the biological activity of IL-17C is dependent on IL-17RA. We also demonstrate that unique antibodies against IL-17RA have the ability to inhibit the biological activity of IL-17C. This and other aspects of the various embodiments of the invention are described.

### BRIEF DESCRIPTION OF THE FIGURES

FIG. 1: IL-17A plus IL-17F inhibition is not as efficacious as IL-17RA inhibition (histological score in a psoriasis mouse model).

FIG. 2A-FIG. 2B: IL-17RA inhibits the expression of genes to a greater extent than IL-17A plus IL-17F inhibition (relative expression levels of two representative genes).

FIG. 3A-FIG. 3F: comparative expression levels of IL-6, GM-CSF, IL-22, IL-1β, IL-12p35, and TNF-α in relation to various IL-17 pathway inhibitors.

FIG. 4A-FIG. 4F: comparative expression levels of MIP-1-α, MIP-1β, IL-8, MMP13, NOS2, and OSM in relation to various IL-17 pathway inhibitors.

FIG. 5A-FIG. 5E: comparative expression levels of G-CSF, IL-23, IL-19, IL-24, and IL-33 in relation to various IL-17 pathway inhibitors.

FIG. 6A-FIG. 6D: comparative expression levels of IL-1F6, IL-1F8, IL-1F9 and IL-1-α in relation to various IL-17 pathway inhibitors.

FIG. 7A-FIG. 7F: comparative expression levels of IL-17A, IL-17B, IL-17C, IL-17D, IL-17E (IL-25), and IL-17F in relation to various IL-17 pathway inhibitors.

FIG. 8A-FIG. 8E: comparative expression levels of IL-17RA, IL-17RB, IL-17RC, IL-17RD, and IL-17RE in relation to various IL-17 pathway inhibitors.

FIG. 9A-FIG. 9F: comparative expression levels of IL-20, S100a8, S100a9, Defb14, Defb4, and Krta6 m relation to various IL-17 pathway inhibitors.

FIG. 10A-FIG. 10F: comparative expression levels of Areg, Btc, Tlr1, Hbegf, and TGFα in relation to various IL-17 pathway inhibitors.

FIG. 11: IL-17C is the only other IL-17 family member upregulated in the skin of mice in a psoriasis model (relative gene expression).

FIG. 12: IL-17RA, IL-17RC and IL-17RE are expressed in the skin of the transgenic mice used in the psoriasis mouse model and the expression level of these receptors is decreased by TP A treatment (relative gene expression).

FIG. 13A-FIG. 13F: IL-17A, IL-17F and IL-17C expression levels increased in the skin of the transgenic mice used in the psoriasis mouse model (relative gene expression).

FIG. 14: IL-17RA, IL-17RE and IL-17RC are expressed in a mouse colon tissue (relative gene expression).

FIG. 15: expression of IL-17A, IL-17F and IL-17C are increased in the colons of colitic mice compared to non-disease mice (relative gene expression).

FIG. 16: expression of IL-17 and IL-17R family members in NHEK cells (relative gene expression).

FIG. 17: IL-17C induced expression of some genes from NHEK cells and that this effect was small compared to IL-17A.

FIG. 18: IL-17C showed synergistic effect with TNF-α in inducing expression of DEFB4, LCN2, S100a8, and G-CSF.

FIG. 19A-FIG. 19B: IL-17C treatment resulted in the induction of G-CSF and LCN2 protein (ELISA results) from NHEK cells and that IL-17C showed an additive effect with TNF-α.

FIG. 20A-FIG. 20B: expression of either IL-17A or IL-17C in mice increased G-CSF in the serum and in cultured splenocytes (Luminex 22-plex data).

FIG. 21A-FIG. 21B: expression of either IL-17A or IL-17C in mice increased G-CSF in the serum and in cultured splenocytes (G-CSF ELISA data).

FIG. 22A-FIG. 22B: IL-17C, IL-17A and G-CSF expression are detectable one day after DNA injection and persist six days after injection.

FIG. 23: protocol for investigating the time course of G-CSF expression after DNA injections to express IL-17A and IL-17C in wild-type mice, IL-17RA deficient mice, and wild-type mice treated with an anti-mouse IL-17RA neutralizing antibody.

FIG. 24A-FIG. 24B: IL-17RA is required for IL-17C-induced G-CSF.

FIG. 25A-FIG. 25B: IL-17A and IL-17C over-expression significantly increased IgA concentrations.

FIG. 26A-FIG. 26B: IL-17A and IL-17C over-expression significantly increased IL-1α concentrations.

FIG. 27: protocol for evaluating IL-17 and IL-17 receptor family member antibodies in response to expression of IL-17A and IL-17C.

FIG. 28A-FIG. 28B: IL-17RA inhibition and IL-17A inhibition significantly reduced IL-17A-induced G-CSF and IL-17RA inhibition significantly reduced IL-17C-induced G-CSF.

FIG. 29: protocol for evaluating IL-17 and IL-17 receptor family member antibodies in response to expression of IL-17A and IL-17C (repeat experiment).

FIG. 30A-FIG. 30B: IL-17RA inhibition and IL-17A inhibition significantly reduced IL-17A-induced G-CSF and IL-17RA inhibition significantly reduced IL-17C-induced G-CSF.

FIG. 31: IL-17C, IL-17A, and IL-17F are highly over-expressed in human lesional psoriasis skin.

FIG. 32A-FIG. 32D: FIG. 32A: IL-17A bound to IL-17RA; FIG. 32B: IL-17C did not bind IL-17RA; FIG. 32C: IL-17A did not bind IL-17RE; FIG. 32D: IL-17C bound to IL-17RE.

FIG. 33A-FIG. 33B: FIG. 33A: IL-17C in the presence of TNF-alpha induced DEFB4 from NHEK cells (Normal Human Epidermal Keratinocytes) in a dose dependent manner; FIG. 33B: IL-17C-induced DEFB4 in NHEK cells was inhibited by IL-17RE-Fc, IL-17RA-Fc and an anti-IL-17RA monoclonal antibody.

FIG. 34: IL-17A induced DEFB4 from NHEK cells in a dose dependent manner.

FIG. 35: Shows the biological activity of IL-17A on NHEK cells, as determined by DFEB4 expression, was inhibited by antibodies against IL-17RA.

FIG. 36: IL-17C induced DEFB4 from NHEK cells in a dose dependent manner.

FIG. 37: Shows the biological activity of IL-17C on NHEK cells, as determined by DFEB4 expression, was inhibited by antibodies against IL-17RA.

### DETAILED DESCRIPTION OF THE INVENTION

The section headings used herein are for organizational purposes only and are not to be construed as limiting the subject matter described.

The present application extends the understanding of the IL-17 family of receptors and ligands and how they interact. The present application teaches that IL-17 Receptor “A” (referred to herein interchangeably as IL-17RA) is required for IL-17C activity. The present application provides evidence that suggests that IL-17RA forms a functional heteromeric receptor complex with IL-17 Receptor “E” (referred to herein interchangeably as IL-17RE), although it is understood that additional subunits or components may also form part of the heteromeric receptor complex. Of particular importance, the present application discloses monoclonal antibodies, preferably human, that specifically bind human IL-17RA and inhibit human IL-17C from activating the human IL-17RA-IL-17RE heteromeric receptor complex.

An art-recognized mouse skin inflammation model psoriasis model) was used to compare inhibition of IL-17RA with inhibition of IL-17A and/or IL-17F in order to better understand which IL-17 family cytokines are driving inflammation through IL-17RA in the skin. Mice with keratinocyte-driven expression of IL-36α (formerly known as IL-1F6), a member of the IL-1 ligand family, when treated with TPA have dramatic skin alterations exhibiting distinct histological similarities with human psoriasis. Importantly, TNF-α, IL-12/23p40 or IL-23-specific neutralizing agents inhibit the observed psoriasis-like skin pathology in this mouse model. IL-17RA inhibition was highly efficacious, comparable to IL-23 inhibition, while IL-17A-specific inhibition consistently provided a partial effect. See Example 1.

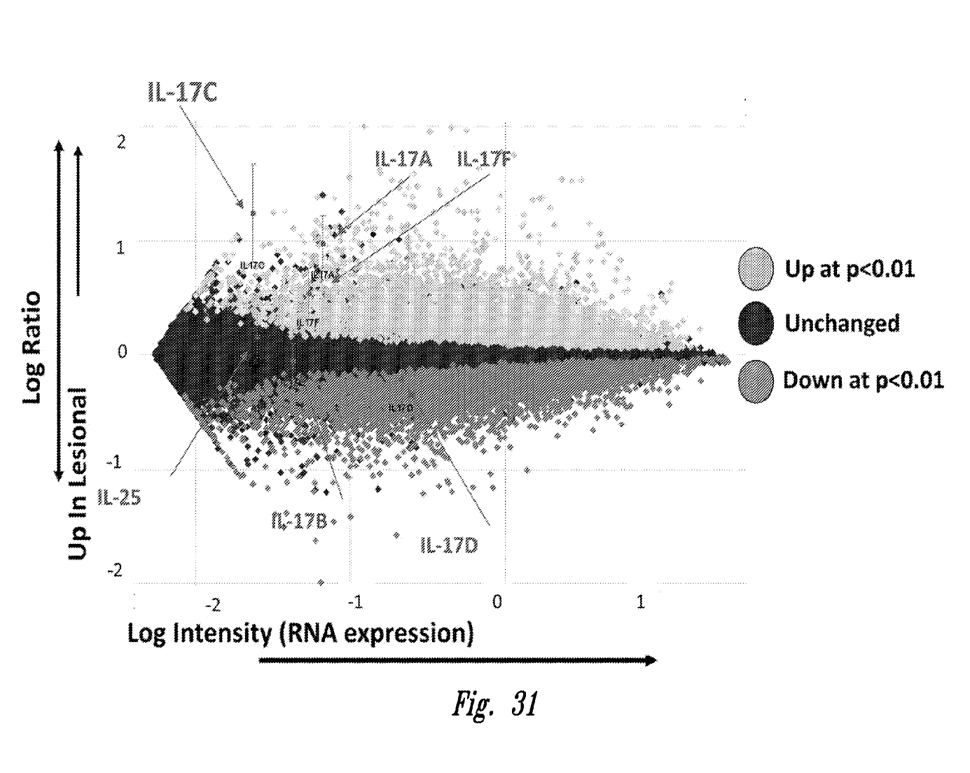

The difference in efficacy did not appear to be due to inhibition of IL-17F in that an IL-17F—specific inhibitor had no effect in this model. In addition, adding IL-17A and IL-17F antibodies at the same time was comparable to IL-17A inhibition alone. IL-25 is not expressed in the skin, however it is possible that another IL-17 family cytokine signals through IL-17RA and contributes to the phenotype. IL-17C is the only other IL-17 family cytokine expressed in the skin in this model, and similar to IL-17A and IL-17F, IL-17C is elevated in human psoriatic lesional tissue (FIG. 31). IL-17C is reported to bind to IL-17RE, but its biological activity is not well understood.

In order to explore the biological activities of IL-17C we over-expressed IL-17C in mice using a hydrodynamic DNA injection method. Mice over-expressing IL-17C exhibited elevated serum G-CSF concentrations. This IL-17C-induced G-CSF response was lost in mice lacking IL-17RA and could be inhibited with an IL-17RA antibody. Antibodies blocking IL-17A, IL-17F, or IL-25 did not significantly affect IL-17C-induced G-CSF. These data suggest that IL-17RA is necessary for IL-17C-induced responses and inhibition of IL-17C in addition to IL-17A could be key to the increased efficacy seen with IL-17RA inhibition compared with IL-17A inhibition in the mouse skin inflammation (psoriasis-like) model.

The present application teaches that IL-17C is elevated in human psoriatic lesional tissue as compared to non-lesional tissue and is also elevated in a mouse model of skin inflammation (i.e., psoriasis). The present application provides evidence showing IL-17C is elevated in a preclinical model of IBD, demonstrating that IL-17C expression is regulated under conditions of excess inflammation. The present application provides evidence showing IL-17C stimulates IL-6, G-CSF, lipocalin-2, DEFB4, S 100a8 and S 100a9 from human epidermal keratinocytes, as well as other genes disclosed in the Examples. This, coupled with what is known in the art, provides a sound basis to predict that blockade of IL-17RA interactions with other IL-17R family members could be therapeutically beneficial to a very wide range of inflammatory diseases including but not limited to rheumatoid arthritis, multiple sclerosis, inflammatory bowel disease, psoriasis, asthma, chronic obstructive pulmonary disease (COPD), and atopic dermatitis.

The present application teaches for the first time that there is sound basis for the proposition that IL-17RA is required for all IL-17 family cytokine activities and that disruption of IL-17RA interactions with other IL-17R subunits inhibits all IL-17 family cytokine activities. Without being bound by theory, we envision an “IL-17RA interacting domain” of IL-17RA that interacts with IL-17RB, IL-17RC, IL-17RD, and IL-17RE.

The present application teaches for the first time that there is sound basis for the proposition that inhibiting human IL-17RA with select IL-17RA-specific neutralizing antibodies, such as those in Table 1 below, inhibits IL-17A, IL-17B, IL-17C, IL-17D, IL-17E (IL-25), IL-17F, IL-17A/F dimer activity. The data presented herein, coupled with what is known in the art, suggests that that IL-17RA-specific neutralizing antibodies, such as those in Table 1 below, bind the “IL-17RA interacting domain” of IL-17RA and prevent the IL-17 receptor subunits from forming a biologically active complex, and thereby unable to become activated upon ligand binding (i.e., any and all IL-17 ligands).

Blocking IL-17RA interactions with other IL-17R family members may be accomplished using antibodies, avimers, peptibodies, or any other molecule (nucleic acid, etc) that inhibits the IL-17RA interacting domain from interacting with other IL-17R family members in the presence or absence of bound IL-17 ligand More specifically, IL-17C may be bound to IL-17RA or IL-17RE and contribute to the formation of a biologically functional IL-17RA-IL-17RE heteromeric receptor complex, and the IL-17RA-IL-17RE antagonists described herein may inhibit this process. Preferred antagonists comprise monoclonal antibodies that specifically bind human IL-17RA. Especially preferred antagonists comprise human monoclonal antibodies that specifically bind human IL-17RA, preferably of the IgG isotype. Specific embodiments are provided in Table 1 below.

The characterization, cloning, and preparation of IL-17RA are described for example in U.S. Pat. No. 6,072,033, issued Jun. 6, 2000, which is incorporated herein by reference in its entirety. The amino acid sequence of the human IL-17RA is shown in SEQ ID NO:10 of U.S. Pat. No. 6,072,033 (GenBank accession number NM_014339). The human IL-17RA has an N-terminal signal peptide with a predicted cleavage site approximately between amino acid 27 and 28. The signal peptide is followed by a 293 amino acid extracellular domain, a 21 amino acid transmembrane domain, and a 525 amino acid cytoplasmic tail. Soluble forms of human IL-17RA (huIL-17RA) that are useful in the methods of the present invention include the extracellular domain (residues 1-320 or residues 28-320 which excludes the signal peptide) or a fragment of the extracellular domain that retains the capacity to bind IL-17A.

IL-17 Receptor E (IL-17RE) is known in the art, such as those disclosed and described in public databases, such as, but not limited to NCBI accession no. Q8NFR9.

In certain embodiments of the invention, it has been discovered that IL-17RA associates with IL-17RE to form a heteromeric receptor complex that is biologically active. Thus, certain aspects of the invention are drawn to agents (e.g., antigen binding proteins, preferably antibodies, as described below) and methods for blocking the association of IL-17RA and IL-17RE, in the presence or absence of bound IL-17 ligand, and thereby preventing a functional receptor complex from being formed and capable of being activated. By preventing a functional receptor complex from being formed, or having an antagonist that binds the IL-17RA-IL-17RE heteromeric receptor complex, this would reduce or prevent receptor activation and reduce the downstream proinflammatory effects of IL-17RA/IL-17RE activation through IL-17 ligands, specifically IL-17C. Such methods and antigen binding proteins would be useful in the treatment of various inflammation and autoimmune disorders that are influenced by the IL-17C/IL-17RE pathway.

Embodiments of the invention are useful for in vitro assays to screen for antagonists or agonists of the IL-17RA-IL-17RE heteromeric receptor complex. Embodiments of the invention are useful for in vitro assays to identify cells expressing the IL-17RA-IL-17RE heteromeric receptor complex. Embodiments of the invention are useful for in vitro assays to identify antagonists of the IL-17C-IL-17RA-IL-17RE heteromeric receptor complex. These are but a few of the many aspects of the various embodiments of the invention described herein.

### 1. IL-17RA-IL-17RE Antagonists

It has been discovered that IL-17RA associates with IL-17RE to form a heteromeric receptor complex that is biologically active. An IL-17RA-IL-17RE heteromeric receptor complex is defined as a physical association (such as, but not limited to, protein-protein interactions) of IL-17RA and IL-17RE proteins and displayed as a heteromeric receptor complex on the extracellular membrane of cells. This heteromeric receptor complex, at a minimum, is required for IL-17RE activation. It is understood that the IL-17RA-IL-17RE heteromeric receptor complex may further comprise additional accessory proteins. IL-17RA-IL-17RE heteromeric receptor complex activation is effectuated through binding of IL-17 ligand family members, specifically IL-17C. IL-17RA-IL-17RE heteromeric receptor complex activation includes, but is not limited to, initiation of intracellular signaling cascade(s) and downstream events such as gene transcription and translation.

Embodiments are directed to antigen binding proteins that inhibit the association of IL-17RA and IL-17RE in forming an IL-17RA-IL-17RE heteromeric receptor complex. An antigen binding protein is preferably an antibody, or fragment thereof, that specifically binds an IL-17RA-IL-17RE heteromeric receptor complex, as variously defined herein. An antigen binding protein may be a peptide or polypeptide that specifically binds the IL-17RA-IL-17RE heteromeric receptor complex. Antigen binding proteins that inhibit the association of IL-17RA and IL-17RE in forming an IL-17RA-IL-17RE biologically functional heteromeric receptor complex are referred to herein as IL-17RA-IL-17RE antagonists. Embodiments of IL-17RA-IL-17RE antagonists may also bind to any part of the IL-17RA-IL-17RE heteromeric receptor complex and inhibit receptor activation by IL-17C. A preferred specific embodiment of an IL-17RA-IL-17RE antagonist is a human monoclonal antibody that specifically binds human IL-17RA and inhibits human IL-17C-mediated activation of the human IL-17RA-IL-17RE heteromeric receptor complex.

“Antigen binding protein” as used herein is a protein that specifically binds an identified target protein, preferably a monoclonal antibody that specifically binds an IL-17RA-IL-17RE heteromeric receptor complex, and more preferably a human monoclonal antibody that specifically binds human IL-17RA and inhibits human IL-17C-mediated activation of the human IL-17RA-IL-17RE heteromeric receptor complex. “Specifically binds” means that the antigen binding protein has higher affinity for the identified target protein than for any other protein. Typically, “specifically binds” mean that the equilibrium dissociation constant is <10⁻⁷ to 10⁻¹¹M, or <10⁻⁸ to <10⁻¹⁰ M, or <10⁻⁹ to <10⁻¹⁰ M.

Activating or activation of a receptor is defined herein as the engagement of one or more intracellular signaling pathway(s) and the transduction of intracellular signaling (i.e., signal transduction) in response to a molecule binding to a membrane-bound receptor, such as but not limited to, a receptor: ligand interaction. Signal transduction, as used herein, is the relaying of a signal by conversion from one physical or chemical form to another; for example, in cell biology, the process by which a cell converts an extracellular signal into a response. Preferred subgenera of the genus of IL-17RA-IL-17RE antagonists comprise antibodies, as variously defined herein; as well as peptides and polypeptides.

“Inhibition” may be measured as a decrease in the association of IL-17RA and IL-17RE proteins in forming a heteromeric receptor complex by at least 10%, 15%, 20%, 25%, 30%, 35%, 40%, 45%, 50%, 55%, 60%, 65%, 70%, 75%, 80%, 85%, 90%, 95%, or 100%. The inhibition of forming a heteromeric receptor complex may be measured by any means known in the art, such as but not limited to the co-immunoprecipitation methods described herein. Other examples include Forster Resonance Energy Transfer (FRET) analysis. In addition, “inhibition” may be measured as a loss of IL-17C activation of an IL-17RA-IL-17RE heteromeric receptor complex as measured by biologically relevant readouts, such as but not limited to upregulated gene transcription and/or gene translation, and/or release of various factors associated with activation of the IL-17RA-IL-17RE heteromeric receptor complex, which includes, but is not limited to: IL-6, IL-8, G-CSF, GM-CSF, TNFα, lipoclin-2, DEFB4, S100a8, and S100a9, as well as any other pathogenic mediator known in the art to be released from human cells expressing IL-17RA-IL-17RE heteromeric receptor complex and activated by IL-17C.

Other embodiments of an IL-17RA-IL-17RE antagonist are directed to IL-17RA-IL-17RE antagonists that bind to IL-17RA, and partially inhibit or fully inhibit association of IL-17RA with IL-17RE and thereby prevent IL-17RA-IL-17RE heteromeric receptor complex formation and activation through binding of IL-17 ligand family members, specifically IL-17C. In one embodiment, the IL-17RA-IL-17RE antagonist need not block the binding of IL-17C from binding to the IL-17RA-IL-17RE heteromeric receptor complex. In alternative embodiments, the IL-17RA-IL-17RE antagonist may block the binding of IL-17C to the IL-17RA-IL-17RE heteromeric receptor complex.

Embodiments of an IL-17RA-IL-17RE antagonist are directed to IL-17RA-IL-17RE antagonists that bind to IL-17RE and partially inhibit or fully inhibit association of IL-17RE with IL-17RA and thereby prevent IL-17RA-IL-17RE heteromeric receptor complex formation and activation through binding of IL-17 ligand family members, specifically IL-17C. In one embodiment, the IL-17RA-IL-17RE antagonist need not block the binding of IL-17C from binding to the IL-17RA-IL-17RE heteromeric receptor complex. In alternative embodiments, the IL-17RA-IL-17RE antagonist may block the binding of IL-17C to the IL-17RA-IL-17RE heteromeric receptor complex.

Additional embodiments of an IL-17RA-IL-17RE antagonist are directed to IL-17RA-IL-17RE antagonists that specifically bind to both IL-17RE and IL-17RA, and partially inhibit or fully inhibit association of IL-17RA with IL-17RE and thereby prevent IL-17RA-IL-17RE heteromeric receptor complex formation and activation through binding of IL-17 ligand family members, specifically IL-17C. In one embodiment, the IL-17RA-IL-17RE antagonist need not block the binding of IL-17C from binding to the IL-17RA-IL-17RE heteromeric receptor. In alternative embodiments, the IL-17RA-IL-17RE antagonists may block the binding of IL-17C to the IL-17RA-IL-17RE heteromeric receptor.

The various embodiments of IL-17RA-IL-17RE antagonists described above include IL-17RA-IL-17RE antagonists that specifically bind to IL-17RA, or IL-17RE, or preferably, both IL-17RA and IL-17RE and sterically inhibit the association of IL-17RA with IL-17RE and thereby prevent IL-17RA-IL-17RE heteromeric receptor complex formation.

Alternatively, the various embodiments of IL-17RA-IL-17RE antagonists described above include IL-17RA-IL-17RE antagonists that bind to IL-17RA, or IL-17RE, or preferably, both IL-17RA and IL-17RE and induce a conformational alteration in IL-17RA, or IL-17RE, or both IL-17RA and IL-17RE and thereby inhibit the association of IL-17RA with IL-17RE and consequently prevent IL-17RA-IL-17RE heteromeric receptor complex formation.

A particularly preferred embodiment of an IL-17RA-IL-17RE antagonist are human monoclonal antibodies that specifically bind human IL-17RA and partially or fully inhibit activation of a human IL-17RA-IL-17RE heteromeric receptor complex through binding of human IL-17C.

### 1.1 IL-17RA-IL-17RE Antagonists: Antibodies

Embodiments of IL-17RA-IL-17RE antagonists comprise antibodies, or fragments thereof, as variously defined herein. Accordingly, the IL-17RA-IL-17RE antagonists include polyclonal antibodies, monoclonal antibodies, bispecific antibodies, diabodies, minibodies, domain antibodies, synthetic antibodies (sometimes referred to herein as “antibody mimetics”), chimeric antibodies, humanized antibodies, fully human antibodies, antibody fusions (sometimes referred to as “antibody conjugates”), as well as fragments thereof.

Particular embodiments of human antibodies that specifically bind human IL-17RA and inhibit IL-17C biological activity include AM12, AM14, AM16, AM17, AM19 and AM22, as well as antibodies, as variously defined herein comprising the respective CDRs of these antibodies, as well as antibodies, as variously defined herein comprising the respective variable heavy and light domains One preferred human antibody is AM14. These antibodies are IL-17RA-IL-17RE antagonists.

TABLE 1

| | | |
|---|---|---|
| Amino acid | SEQ ID NO: 1 | QVQLVQSGAEVKKPGASVKVSCKASGYTLT |
| sequence | | SYGISWVRQAPGQGLEWMGWISTYKGNTNY |
| AMH12Vh | | AQKLQGRVTMTTDTSTSTAYMELRSLRSDD |
| | | TAVYYCARKQLVFDYWGQGTLVTVSS |
| Amino acid | SEQ ID NO: 2 | EIVMTQSPATLSVSPGERATLSCRASQSISSSL |
| sequence | | AWYQQKPGQAPRLLIYGASTRATGIPARFSG |
| AML12 Vl | | SGSGTEFTLTISSLQSENFAVYYCQQYDNWP |
| | | LTFGGGTKVEIK |
| Amino acid | SEQ ID NO: 3 | QVQLVQSGAEVKKPGASVKVSCKASGYTFT |
| sequence | | RYGISWVRQAPGQGLEWMGWISTYSGNTNY |
| AMH14 Vh | | AQKLQGRVTMTTDTSTSTAYMELRSLRSDD |
| | | TAVYYCARRQLYFDYWGQGTLVTVSS |
| Amino acid | SEQ ID NO: 4 | EIVMTQSPATLSVSPGERATLSCRASQSVSSN |
| sequence | | LAWFQQKPGQAPRPLIYDASTRATGVPARFS |
| AML14 Vl | | GSGSGTDFTLTISSLQSEDFAVYYCQQYDNW |
| | | PLTFGGGTKVEIK |
| Amino acid | SEQ ID NO: 5 | QVQLVQSGAEVKKPGASVKVSCKASGYTFT |
| sequence | | SYGISWVRQAPGQGLEWMGWISAYNGNTK |
| AMH16 Vh | | YAQKLQGRVTMTTDTSTSTVYMELRSLRSD |
| | | DTAVYYCARKQLVFDYWGQGTLVTVSS |
| Amino acid | SEQ ID NO: 6 | EIVMTQSPATLSVSPGERATLSCRASQSISTSL |
| sequence | | AWYQQKPGQAPRLLIYGTSTRATGIPARARFSG |
| AML16 Vl | | SGSGTEFTLTISSLQSEDFAVYFCQQYDIWPL |
| | | TFGGGTKVEIK |
| Amino acid | SEQ ID NO: 7 | QVQLVQSGAEVKKPGAAVKVSCKATGYTLT |
| sequence | | SYGISWVRQAPGQGLEWMGWISAYSGNTKY |
| AMH17Vh | | AQKLQGRVTMTTDTSTSTAYMELRSLRSDD |
| | | TAVYYCARKQLVFDYWGQGTLVTVSS |
| Amino acid | SEQ ID NO: 8 | EIVMTQSPATLSVSPGERATLSCRASQSVSSN |
| sequence | | LAWYQQKPGQAPRLLIYGASTRATGIPARFS |
| AML 17Vl | | GSGSGTEFTLTISSLQSEDFAVYSCQQYDNW |
| | | PLTFGGGTKVEIK |
| Amino acid | SEQ ID NO: 9 | QVQLVQSGAEVKKPGASVKVSCKASGYTLT |
| sequence | | SYGISWVRQAPGQGLEWMGWISAYSGNTKY |
| AMH19Vh | | AQKFQGRVTMTTDTSTSTAYMELRSLRSDD |
| | | TAVYYCARRQLALDYWGQGTLVTVSS |
| Amino acid | SEQ ID NO: 10 | EIVMTQSPATLSVSPGERATLSCRASQSISSNL |
| sequence | | AWYQQKPGQAPRLLIYGASTRATGIPARFSD |
| AML 19Vl | | NGSGTEFTLTISSLQSEDFAVYFCQQYDTWPL |
| | | TFGGGTKVEIK |
| Amino acid | SEQ ID NO: 11 | QVQLVQSGAEVKKPGASVKVSCKASGYTFT |
| sequence | | RYGISWVRQAPGQGLEWMGWISAYSGNTN |
| AMH22Vh | | YAQKLQGRVTMTTDTSTSTAYMELRSLRSD |
| | | DTAVYYCARRQLYFDYWGQGTLVTVSS |
| Amino acid | SEQ ID NO: 12 | EIVMTQSPATLSVSPGERVTLSCRASQSVSSN |
| sequence | | LAWFQQKPGQAPRPLIYDASTRAAGIPARFS |
| AML22Vl | | GSGSGTDFTLTISSLQSEDFAVYYCQQYDNW |
| | | PLTFGGGTKVEIK |
| Amino acid | SEQ ID NO: 13 | SYGIS |
| sequence of | | |
| CDR 1 of | | |
| AM_{H}12Vh | | |
| Amino acid | SEQ ID NO: 14 | WISTYKGNTNYAQKLQG |
| sequence of | | |
| CDR 2 of | | |
| AM_{H}12Vh | | |
| Amino acid | SEQ ID NO: 15 | KQLVFDY |
| sequence of | | |
| CDR 3 of | | |
| AM_{H}12Vh | | |
| Amino acid | SEQ ID NO: 16 | RYGIS |
| sequence of | | |
| CDR 1 of | | |
| AM_{H}14 Vh | | |
| Amino acid | SEQ ID NO: 17 | WISTYSGNTNYAQKLQG |
| sequence of | | |
| CDR 2of | | |
| AM_{H}14 Vh | | |
| Amino acid | SEQ ID NO: 18 | RQLYFDY |
| sequence of | | |
| CDR 3 of | | |
| AM_{H}14 Vh | | |
| Amino acid | SEQ ID NO: 19 | SYGIS |
| sequence of | | |
| CDR 1 of | | |
| AM_{H}16Vh | | |
| Amino acid | SEQ ID NO: 20 | WISAYNGNTKYAQKLQG |
| sequence of | | |
| CDR 2 of | | |
| AM_{H}16 Vh | | |
| Amino acid | SEQ ID NO: 21 | KQLVFDY |
| sequence of | | |
| CDR 3 of | | |
| AM_{H}16Vh | | |
| Amino acid | SEQ ID NO: 22 | SYGIS |
| sequence of | | |
| CDR 1 of | | |
| AM_{H}17Vh | | |
| Amino acid | SEQ ID NO: 23 | WISAYSGNTKYAQKLQG |
| sequence of | | |
| CDR 2of | | |
| AM_{H}17V | | |
| Amino acid | SEQ ID NO: 24 | KQLVFDY |
| sequence of | | |
| CDR3 of | | |
| AM_{H}17Vh | | |
| Amino acid | SEQ ID NO: 25 | SYGIS |
| sequence of | | |
| CDR 1 of | | |
| AM_{H}19 Vh | | |
| Amino acid | SEQ ID NO: 26 | WISAYSGNTKYAQKLQG |
| sequence of | | |
| CDR 2of | | |
| AM_{H}19Vh | | |
| Amino acid | SEQ ID NO: 27 | RQLALDY |
| sequence of | | |
| CDR3 of | | |
| AM_{H}19 Vh | | |
| Amino acid | SEQ ID NO: 28 | WISAYSGNTNYAQKLQG |
| sequence of | | |
| CDR 2 of | | |
| AM_{H}22Vh | | |
| Amino acid | SEQ ID NO: 29 | RQLYFDY |
| sequence of | | |
| CDR 3 of | | |
| AM_{H}22Vh | | |
| Amino acid | SEQ ID NO: 30 | RASQSISSSLA |
| sequence of | | |
| CDR 1 of | | |
| AM_{L}12Vl | | |
| Amino acid | SEQ ID NO: 31 | GASTRAT |
| sequence of | | |
| CDR 2 of | | |
| AM_{L}12 Vl | | |
| Amino acid | SEQ ID NO: 32 | QQYDNWPLT |
| sequence of | | |
| CDR 3 of | | |
| AM_{L}12 Vl | | |
| Amino acid | SEQ ID NO: 33 | RASQSVSSNLA |
| sequence of | | |
| CDR 1 of | | |
| AM_{L}14 Vl | | |
| Amino acid | SEQ ID NO: 34 | DASTRAT |
| sequence of | | |
| CDR 2of | | |
| AM_{L}14 Vl | | |
| Amino acid | SEQ ID NO: 35 | QQYDNWPLT |
| sequence of | | |
| CDR 3 of | | |
| AM_{L}14 Vl | | |
| Amino acid | SEQ ID NO: 36 | RASQSISTSLA |
| sequence of | | |
| CDR 1 of | | |
| AM_{L}16Vl | | |
| Amino acid | SEQ ID NO: 37 | GTSTRAT |
| sequence of | | |
| CDR 2 of | | |
| AM_{L}16 Vl | | |
| Amino acid | SEQ ID NO: 38 | QQYDIWPLT |
| sequence of | | |
| CDR 3 of | | |
| AM_{L}16 Vl | | |
| Amino acid | SEQ ID NO: 39 | RASQSVSSNLA |
| sequence of | | |
| CDR 1 of | | |
| AM_{L}17 Vl | | |
| Amino acid | SEQ ID NO: 40 | GASTRAT |
| sequence of | | |
| CDR2of | | |
| AM_{L}17 Vl | | |
| Amino acid | SEQ ID NO: 41 | QQYDNWPLT |
| sequence of | | |
| CDR 3 of | | |
| AM_{L}17 Vl | | |
| Amino acid | SEQ ID NO: 42 | RASQSISSNLA |
| sequence of | | |
| CDR 1 of | | |
| AM_{L}19 Vl | | |
| Amino acid | SEQ ID NO: 43 | GASTRAT |
| sequence of | | |
| CDR 2 of | | |
| AM_{L}19 Vl | | |
| Amino acid | SEQ ID NO: 44 | QQYDTWPLT |
| sequence of | | |
| CDR 3 of | | |
| AM_{L}19 Vl | | |
| Amino acid | SEQ ID NO: 45 | RASQSVSSNLA |
| sequence of | | |
| CDR 1 of | | |
| AM_{L}22 Vl | | |
| Amino acid | SEQ ID NO: 46 | DASTRAA |
| sequence of | | |
| CDR2 of | | |
| AM_{L}22 Vl | | |
| Amino acid | SEQ ID NO: 47 | QQYDNWPLT |
| sequence of | | |
| CDR 3 of | | |
| AM_{L}22 Vl | | |
| Amino acid | SEQ ID NO: 48 | MEWTWRVLFLVAAATGAHSQVQLVQSGAE |
| sequence | | VKKPGASVKVSCKASGYTFTRYGISWVRQA |
| AM_{H}14 | | PGQGLEWMGWISTYSGNTNYAQKLQGRVT |
| full- | | MTTDTSTSTAYMELRSLRSDDTAVYYCARR |
| length | | QLYFDYWGQGTLVTVSSASTKGPSVFPLAPC |
| heavy | | SRSTSESTAALGCLVKDYFPEPVTVSWNSGA |
| chain | | LTSGVHTFPAVLQSSGLYSLSSVVTVPSSNFG |
| | | TQTYTCNVDHKPSNTKVDKTVERKCCVECP |
| | | PCPAPPVAGPSVFLFPPKPKDTLMISRTPEVT |
| | | CVVVDVSHEDPEVQFNWYVDGVEVHNAKT |
| | | KPREEQFNSTFRVVSVLTVVHQDWLNGKEY |
| | | KCKVSNKGLPAPIEKTISKTKGQPREPQVYTL |
| | | PPSREEMTKNQVSLTCLVKGFYPSDIAVEWE |
| | | SNGQPENNYKTTPPMLDSDGSFFLYSKLTVD |
| | | KSRWQQGNVFSCSVMHEALHNHYTQKSLSL |
| | | SPGK |
| Amino acid | SEQ ID NO: 49 | EIVMTQSPATLSVSPGERATLSCRASQSVSSN |
| sequence | | LAWFQQKPGQAPRPLIYDASTRATGVPARFS |
| AM_{L}14 | | GSGSGTDFTLTISSLQSEDFAVYYCQQYDNW |
| full- | | PLTFGGGTKVEIKRTVAAPSVFIFPPSDEQLKS |
| length | | GTASVVCLLNNFYPREAKVQWKVDNALQSG |
| light | | NSQESVTEQDSKDSTYSLSSTLTLSKADYEK |
| chain | | HKVYACEVTHQGLSSPVTKSFNRGEC |

IL-17RA-IL-17RE antagonist antibodies may also comprise single-domain antibodies that comprise dimers of two heavy chains and include no light chains, such as those found in camels and llamas (see, for example Muldermans, et al., 2001, J. Biotechnol. 74:277-302; Desmyter, et al., 2001, J. Biol. Chem. 276:26285-26290).

IL-17RA-IL-17RE antagonist antibodies may comprise a tetramer, or fragments thereof. Each tetramer is typically composed of two identical pairs of polypeptide chains, each pair having one “light” (typically having a molecular weight of about 25 kDa) and one “heavy” chain (typically having a molecular weight of about 50-70 kDa). The amino-terminal portion of each chain includes a variable region is primarily responsible for antigen recognition. The carboxy-terminal position of each chain defines a constant region primarily responsible for effector function Human light chains are classified as kappa and lambda light chains Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. IL-17RA-IL-17RE antagonist antibodies include all such isotypes. For exemplary purposes, antibody fragments include but are not limited to F(ab), F(ab′), F(ab′)2, Fv, and single chain Fv fragments (scfv), as well as single-chain antibodies. IL-17RA-IL-17RE antagonist antibodies may comprise any of the foregoing examples.

The structure of antibodies is well known in the art and need not be reproduced here, but by way of example, the variable regions of the heavy and light chains typically exhibit the same general structure of relatively conserved framework regions (FR) joined by three hypervariable regions, also called complementarity determining regions or CDRs. The CDRs are the hypervariable regions of an antibody (or antigen binding protein, as outlined herein), that are responsible for antigen recognition and binding. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. In some embodiments, the assignment of amino acids to each domain may be in accordance with the definitions of Kabat Sequences of Proteins of Immunological Interest. See, Chothia, et al., 1987, J. Mol. Biol. 196:901-917; Chothia, et al., 1989, Nature 342:878-883.

A “complementary determining region” or “CDR,” as used herein, refers to a binding protein region that constitutes the major surface contact points for antigen binding. A binding protein of the invention may have six CDRs, for example one heavy chain CDR1 (“CDRH1”), one heavy chain CDR1 (“CDRH1”), one heavy chain CDR2 (“CDRH2”), one heavy chain CDR3 (“CDRH3”), one light chain CDR1 (“CDRL1”), one light chain CDR2 (“CDRL2”), one light chain CDR3 (“CDRL3”). CDRH1 typically comprises about five (5) to about seven (7) amino acids, CDRH2 typically comprises about sixteen (16) to about nineteen (19) amino acids, and CDRH3 typically comprises about three (3) to about twenty five (25) amino acids. CDRL1 typically comprises about ten (10) to about seventeen (17) amino acids, CDRL2 typically comprises about seven (7) amino acids, and CDRL3 typically comprises about seven (7) to about ten (10) amino acids

At a minimum, an IL-17RA-IL-17RE antagonist antibody comprises all or part of a light or heavy chain variable region, or all or part of both a light and heavy chain variable region that specifically binds to IL-17RA, or IL-17RE, or both IL-17RA and IL-17RE. Examples of fragments (i.e., “part”) of variable regions comprise the CDRs. Stated differently, at a minimum, an IL-17RA-IL-17RE antagonist antibody comprises at least one CDR of a variable region, wherein the CDR specifically binds IL-17RA, or IL-17RE, or both IL-17RA and IL-17RE. In alternative embodiments, an IL-17RA-IL-17RE antagonist antibody comprises at least two, or at least three, or at least four, or at least five, or at least all six CDRs of a/the variable region(s), wherein at least one of the CDRs specifically binds IL-17RA, or IL-17RE, or both IL-17RA and IL-17RE. The CDR may be from a heavy or light chain, and may be one of any of the three CDRs within each chain, that is, the CDRs are each independently selected from CDRH1, CDRH2, CDRH3, CDRL1, CDRL2 and CDRL3. Embodiments of the IL-17RA-IL-17RE antagonist antibodies may comprise a scaffold structure into which useful CDR(s) are grafted. Some embodiments include human scaffold components for humanized antibodies. In one embodiment, the scaffold structure is a traditional, tetrameric antibody structure. Thus, embodiments of the IL-17RA-IL-17RE antagonist antibodies may include the additional components such as framework, J and D regions, constant regions, etc. that make up a heavy or light chain Embodiments of the IL-17RA-IL-17RE antagonist antibodies may comprise antibodies that have a modified Fc domain, referred to as an Fc variant. An “Fc variant” refers to a molecule or sequence that is modified from a native Fc but still comprises a binding site for the salvage receptor, FcRn. Other examples of an “Fc variant” includes a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Embodiments of IL-17RA-IL-17RE antagonist antibodies comprise human monoclonal antibodies Human monoclonal antibodies directed against human IL-17RA, or IL-17RE, or both IL-17RA and IL-17RE may be made using any known methods known in the art, such as but not limited to XenoMouse™ technology (see, for example U.S. Pat. Nos. 6,114,598; 6,162,963; 6,833,268; 7,049,426; 7,064,244; Green et al, 1994, Nature Genetics 7:13-21; Mendez et al., 1997, Nature Genetics 15:146-156; Green and Jakobovitis, 1998, J. Ex. Med. 188:483-495). Other examples of making fully human antibodies include UltiMab Human Antibody Development System™ and Trans-Phage Technology™ (Medarex Corp., Princeton, N.J.), phage-display technologies, ribosome-display technologies (see for example Cambridge Antibody Technology, Cambridge, UK), as well as any other method known in the art. Preferred embodiments include human monoclonal antibodies that specifically bind to both IL-17RE and IL-17RA that partially or fully inhibit activation and/or binding by IL-17C.

Certain embodiments of IL-17RA-IL-17RE antagonist antibodies comprise chimeric and humanized antibodies, or fragments thereof. In general, both chimeric antibodies and humanized antibodies refer to antibodies that combine regions from more than one species. For example, chimeric antibodies traditionally comprise variable region(s) from a non-human species and the constant region(s) from a human Humanized antibodies generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is well known in the art (see, for example Jones, 1986, Nature 321:522-525; Verhoeyen et al., 1988, Science 239: 1534-1536) Humanized antibodies can also be generated using mice with a genetically engineered immune system or by any other method or technology known in the art (see for example Roque, et al., 2004, Biotechnol. Prog. 20:639-654). In some embodiments, the CDRs are human, and thus both humanized and chimeric antibodies in this context can include some non-human CDRs; for example, humanized antibodies may be generated that comprise the CDRH3 and CDRL3 regions, with one or more of the other CDR regions being of a different special origin.

In one embodiment, the IL-17RA-IL-17RE antagonist antibodies comprise a multispecific antibody. These are antibodies that bind to two (or more) different antigens. An example of a bispecific antibody known in the art are “diabodies”. Diabodies can be manufactured in a variety of ways known in the art, e.g., prepared chemically or from hybrid hybridomas (Holliger and Winter, 1993, Current Opinion Biotechnol. 4:446-449). A specific embodiment of a multispecific IL-17RA-IL-17RE antagonist antibody is an antibody that has the capacity to bind to both IL-17RA and IL-17RE.

In alternative embodiments, the IL-17RA-IL-17RE antagonist antibodies comprise a minibody. Minibodies are minimized antibody-like proteins comprising a scFv joined to a CH3 domain (see, for example Hu, et al., 1996, Cancer Res. 56:3055-3061).

In alternative embodiments, the IL-17RA-IL-17RE antagonist antibodies comprise a domain antibody; for example those described in U.S. Pat. No. 6,248,516. Domain antibodies (dAbs) are functional binding domains of antibodies, corresponding to the variable regions of either the heavy (VH) or light (VL) chains of human antibodies. dAbs have a molecular weight of approximately 13 kDa, or less than one-tenth the size of a full antibody. dAbs are well expressed in a variety of hosts including bacterial, yeast, and mammalian cell systems. In addition, dAbs are highly stable and retain activity even after being subjected to harsh conditions, such as freeze-drying or heat denaturation. See, for example, U.S. Pat. Nos. 6,291,158; 6,582,915; 6,593,081; 6,172,197; US Serial No. 2004/0110941; European Patent 0368684; U.S. Pat. No. 6,696,245, WO04/058821, WO04/003019 and WO03/002609.

As mentioned previously, the IL-17RA-IL-17RE antagonist antibodies may comprise an antibody fragment, i.e., a fragment of any of the antibodies mentioned herein that retain binding specificity to IL-17RA, or IL-17RE, or both IL-17RA and IL-17RE. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (see for example Ward, et al., 1989, Nature 341:544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (see, for example Bird, et al., 1988 Science 242:423-426; Huston, et al., 1988, Proc. Natl. Acad. Sci. 85:5879-5883), (viii) bispecific single chain Fv dimers, and (ix) “diabodies” or “triabodies”, multivalent or multispecific fragments constructed by gene fusion (see, for example, Tomlinson, et. al., 2000, Methods Enzymol. 326:461-479; WO94/13804; Holliger, et al., 1993, Proc. Natl. Acad. Sci. 90:6444-6448). The antibody fragments may be modified. For example, the molecules may be stabilized by the incorporation of disulphide bridges linking the VH and VL domains (see, for example, Reiter, et al., 1996, Nature Biotech. 14:1239-1245) Again, as outlined herein, the non-CDR components of these fragments are preferably human sequences.

In further embodiments, the IL-17RA-IL-17RE antagonist antibodies comprise an antibody fusion protein (sometimes referred to herein as an “antibody conjugate”). The conjugate partner can be proteinaceous or non-proteinaceous; the latter generally being generated using functional groups on the antigen binding protein (see the discussion on covalent modifications of the antigen binding proteins) and on the conjugate partner. For example linkers are known in the art; for example, homo- or hetero-bifunctional linkers as are well known (see, for example, 1994 Pierce Chemical Company catalog, technical section on cross-linkers, pages 155-200, incorporated herein by reference). Suitable conjugates include, but are not limited to, labels as described below, drugs and cytotoxic agents including, but not limited to, cytotoxic drugs (e.g., chemotherapeutic agents) or toxins or active fragments of such toxins. Suitable toxins and their corresponding fragments include diptheria A chain, exotoxin A chain, ricin A chain, abrin A chain, curcin, crotin, phenomycin, enomycin and the like. Cytotoxic agents also include radiochemicals made by conjugating radioisotopes to antigen binding proteins, or binding of a radionuclide to a chelating agent that has been covalently attached to the antigen binding protein. Additional embodiments utilize calicheamicin, auristatins, geldanamycin and maytansine.

In one embodiment, the IL-17RA-IL-17RE antagonist antibodies comprise an antibody analog, sometimes referred to as “synthetic antibodies.” For example, a variety of alternative protein scaffolds or artificial scaffolds may be grafted with CDRs from IL-17RA-IL-17RE antagonist antibodies. Such scaffolds include, but are not limited to, mutations introduced to stabilize the three-dimensional structure of the binding protein as well as wholly synthetic scaffolds consisting for example of biocompatible polymers. See, for example, Korndorfer, et al., 2003, Proteins: Structure, Function, and Bioinformatics, Volume 53, Issue 1: 121-129; Roque, et al., 2004, Biotechnol. Prag. 20:639-654. In alternative embodiments the IL-17RA-IL-17RE antagonist antibodies may comprise peptide antibody mimetics, or “PAMs”, as well as antibody mimetics utilizing fibronection components as a scaffold.

### 1.2 IL-17RA-IL-17RE Antagonists: Peptides/Polypeptides

Embodiments of IL-17RA-IL-17RE antagonists comprise proteins in the form of peptides and polypeptides that specifically bind to IL-17RA, or IL-17RE, or both IL-17RA and IL-17RE that inhibit the association of IL-17RA and IL-17RE in forming an IL-17RA-IL-17RE heteromeric receptor complex. Embodiments include recombinant IL-17RA-IL-17RE antagonists. A “recombinant protein” is a protein made using recombinant techniques, i.e., through the expression of a recombinant nucleic acid using methods known in the art.

A “peptide,” as used herein refers to molecules of 1 to 100 amino acids. Exemplary peptides that bind to IL-17RA, or IL-17RE, or both IL-17RA and IL-17RE that inhibit the association of IL-17RA and IL-17RE in forming an IL-17RA-IL-17RE heteromeric receptor complex or inhibit IL-17RA-IL-17RE heteromeric receptor complex signaling may comprise those generated from randomized libraries. For example, peptide sequences from fully random sequences (e.g., selected by phage display methods or RNA-peptide screening) and sequences in which one or more residues of a naturally occurring molecule is replaced by an amino acid residue not appearing in that position in the naturally occurring molecule. Exemplary methods for identifying peptide sequences include phage display, E. coli display, ribosome display, RNA-peptide screening, chemical screening, and the like.

By “protein,” as used herein, is meant at least two covalently attached amino acids, which includes proteins, polypeptides, oligopeptides and peptides. In some embodiments, the two or more covalently attached amino acids are attached by a peptide bond. The protein may be made up of naturally occurring amino acids and peptide bonds, for example when the protein is made recombinantly using expression systems and host cells, as outlined below. Alternatively, in some embodiments (for example when proteinaceous candidate agents are screened for the ability to inhibit IL-17RA and IL-17RE association) the protein may include synthetic amino acids (e.g., homophenylalanine, citrulline, ornithine, and norleucine), or peptidomimetic structures, i.e., “peptide or protein analogs”, such as peptoids (see, Simon et al., 1992, Proc. Natl. Acad. Sci. U.S.A. 89:9367, incorporated by reference herein), which can be resistant to proteases or other physiological and/or storage conditions. Such synthetic amino acids may be incorporated in particular when the antigen binding protein is synthesized in vitro by conventional methods well known in the art. In addition, any combination of peptidomimetic, synthetic and naturally occurring residues/structures can be used. “Amino acid” also includes imino acid residues such as proline and hydroxyproline. The amino acid “R group” or “side chain” may be in either the (L)- or the (S)-configuration. In a specific embodiment, the amino acids are in the (L)- or (S)-configuration.

In some embodiments, the antigen binding proteins of the invention are isolated proteins or substantially pure proteins. An “isolated” protein is unaccompanied by at least some of the material with which it is normally associated in its natural state, preferably constituting at least about 5%, more preferably at least about 50% by weight of the total protein in a given sample. A “substantially pure” protein comprises at least about 75% by weight of the total protein, with at least about 80% being specific, and at least about 90% being particularly specific. The definition includes the production of an antigen binding protein from one organism in a different organism or host cell. Alternatively, the protein may be made at a significantly higher concentration than is normally seen, through the use of an inducible promoter or high expression promoter, such that the protein is made at increased concentration levels.

### 2.0 IL-17RA-IL-17RE Antigen Binding Proteins: Modifications

As mentioned above, IL-17RA-IL-17RE antigen binding proteins include IL-17RA-IL-17RE antagonists, which includes, but is not limited to, antibodies, peptides, and polypeptides. Alternative embodiments of IL-17RA-IL-17RE antigen binding proteins (e.g., IL-17RA-IL-17RE antagonists) comprise covalent modifications of IL-17RA-IL-17RE antigen binding proteins. The antibodies in Table 1 are embodiments of IL-17RA-IL-17RE antagonistic antibodies and may be modified as described in this section. Such modifications may be done post-translationally. For example, several types of covalent modifications of the IL-17RA-IL-17RE antigen binding proteins are introduced into the molecule by reacting specific amino acid residues of the antigen binding protein with an organic derivatizing agent that is capable of reacting with selected side chains or the N- or C-terminal residues. The following represent examples of such modifications to the IL-17RA-IL-17RE antigen binding proteins.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in IL-17RAdioimmunoassay, the chloramine T method described above being suitable. Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for cross linking IL-17RA-IL-17RE antagonists to a water-insoluble support matrix or surface for use in a variety of methods. Commonly used crosslinking agents include, e.g., 1,1-bis(diazoacetyl)-2-phenylethane, glutaraldehyde, Nhydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4, 195, 128; 4,247,642; 4,229,537; and 4,330,440 are employed for protein immobilization Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of praline and lysine, phosphorylation of hydroxyl groups of seryl or threonyl residues, methylation of the a-amino groups of lysine, arginine, and histidine side chains (T. E. Creighton, Proteins: Structure and Molecular Properties, W. H. Freeman & Co., San Francisco, pp. 79-86 [1983]), acetylation of the N-terminal amine, and amidation of any C-terminal carboxyl group. Another type of covalent modification of the IL-17RA-IL-17RE antagonists included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Glycosylation of polypeptides is typically either N-linked or O-linked N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used. Addition of glycosylation sites to the IL-17RA-IL-17RE antagonists is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the IL-17RA-IL-17RE antagonists is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston, 1981, CRC Crit. Rev. Biochem., pp. 259-306.

Removal of carbohydrate moieties present on the starting IL-17RA-IL-17RE antagonists may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118: 131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Another type of covalent modification of the IL-17RA-IL-17RE antagonists comprises linking the antigen binding protein to various nonproteinaceous polymers, including, but not limited to, various polyols such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antigen binding protein to facilitate the addition of polymers such as PEG.

In some embodiments, the covalent modification of the IL-17RA-IL-17RE antagonists of the invention comprises the addition of one or more labels. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antigen binding protein via spacer arms of various lengths to reduce potential steric hindrance. Various methods for labelling proteins are known in the art and may be used in performing the present invention. Specific labels include optical dyes, including, but not limited to, chromophores, phosphors and fluorophores, with the latter being specific in many instances. Fluorophores can be either “small molecule” fluores, or proteinaceous fluores.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described m Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

Covalent modifications of IL-17RA-IL-17RE antagonists are included within the scope of this invention, and are generally, but not always, done post-translationally. For example, several types of covalent modifications of the IL-17RA-IL-17RE antagonists are introduced into the molecule by reacting specific amino acid residues of the IL-17RA-IL-17RE antagonists with an organic derivatizing agent that is capable of reacting with selected side chains or the N- or C-terminal residues.

In some embodiments, the covalent modification of the antigen binding proteins of the invention comprises the addition of one or more labels. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antigen binding protein via spacer arms of various lengths to reduce potential steric hindrance. Various methods for labelling proteins are known in the art and may be used in performing the present invention.

Specific labels include optical dyes, including, but not limited to, chromophores, phosphors and fluorophores, with the latter being specific in many instances. Fluorophores can be either “small molecule” fluores, or proteinaceous fluores. By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methylcoumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and Rphycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 119; Stauber, 1998, Biotechniques 24:462-471; Heim et al., 25 1996, Curr. Biol. Q.: 178-182), enhanced yellow fluorescent protein (EYFP, Clontech Laboratories, Inc.), luciferase (Ichiki et al., 1993, J. Immunol. 150:5408-5417), galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

### 3.0 Methods of Use

Select IL-17RA-specific neutralizing antibodies, specifically those in Table 1, can be used in a method of inhibiting IL-17A, IL-17B, IL-17C, IL-17D, IL-17E (IL-25), IL-17F, and IL-17A/F dimer activity.

Additional embodiments include methods of inhibiting IL-17RA and/or IL-17RE activation in cells expressing IL-17RA and IL-17RE using one or more of the IL-17RA-IL-17RE antagonists described herein. For example, a method of inhibiting IL-17RA and/or IL-17RE activation in cells expressing IL-17RA and IL-17RE comprises exposing said cells to an IL-17RA-IL-17RE antagonist, wherein the IL-17RA-IL-17RE antagonist binds IL-17RA and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein.

Additional embodiments include methods of inhibiting IL-17RA and/or IL-17RE activation in cells expressing IL-17RA and IL-17RE using one or more of the IL-17RA-IL-17RE antagonists described herein. For example, a method of inhibiting IL-17RA and/or IL-17RE activation in cells expressing IL-17RA and IL-17RE comprises exposing said cells to an IL-17RA-IL-17RE antagonist, wherein the IL-17RA-IL-17RE antagonist binds IL-17RE and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C. In one embodiment, the IL-17RA-IL-17RE antagonist need not block the binding of IL-17C from binding to IL-17RE. In alternative embodiments, the IL IL-17RA-IL-17RE antagonist may block the binding of IL-17C to IL-17RE.

Additional embodiments comprise a method wherein said IL-17RA-IL-17RE antagonist is an antibody, as defined herein. Additional embodiments include methods of inhibiting IL-17RA and/or IL-17RE activation in cells expressing IL-17RA and IL-17RE using one or more of the IL-17RA-IL-17RE antagonists described herein. For example, a method of inhibiting IL-17RA and/or IL-17RE activation in cells expressing IL-17RA and IL-17RE comprises exposing said cells to an IL-17RA-IL-17RE antagonist, wherein the IL-17RA-IL-17RE antagonist binds both IL-17RA and IL-17RE and partially inhibit or fully inhibits association of IL-17RE with IL-17RA and thereby preventing IL-17RA-IL-17RE heteromeric receptor complex formation and activation through binding of IL-17 ligand family members, such as but not limited to IL-17C. In one embodiment, the IL-17RA-IL-17RE antagonist need not block the binding of IL-17C from binding IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein.

Additional embodiments include methods of inhibiting IL-17RA and/or IL-17RE activation in cells expressing IL-17RA and IL-17RE in vivo using one or more of the IL-17RA-IL-17RE antagonists described herein. For example, a method of inhibiting IL-17RA and/or IL-17RE activation in cells expressing IL-17RA and IL-17RE in vivo comprises exposing said cells to an IL-17RA-IL-17RE antagonist, wherein the IL-17RA-IL-17RE antagonist binds IL-17RA and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition.

Additional embodiments include methods of inhibiting IL-17RA and/or IL-17RE activation in cells expressing IL-17RA and IL-17RE in vivo using one or more of the IL-17RA-IL-17RE antagonists described herein. For example, a method of inhibiting IL-17RA and/or IL-17RE activation in cells expressing IL-17RA and IL-17RE in vivo comprises exposing said cells to an IL-17RA-IL-17RE antagonist, wherein the IL-17RA-IL-17RE antagonist binds IL-17RE and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-1 ?RE antagonist is an antibody, as defined herein. Additional embodiments comprise a method wherein said IL-17RA-IL-17RE antagonist is an antibody, as defined herein, and the antibody is in the form of a pharmaceutical composition.

Additional embodiments include methods of inhibiting IL-17RA and/or IL-17RE activation in cells expressing IL-17RA and IL-17RE in vivo using one or more of the IL-17RA-IL-17RE antagonists described herein. For example, a method of inhibiting IL-17RA and/or IL-17RE activation in cells expressing IL-17RA and IL-17RE in vivo comprises exposing said cells to an IL-17RA-IL-17RE antagonist, wherein the IL-17RA-IL-17RE antagonist binds both IL-17RA and IL-17RE and partially inhibit or fully inhibits association of IL-17RE with IL-17RA and thereby preventing IL-17RA-IL-17RE heteromeric receptor complex formation and activation through binding of IL-17 ligand family members, such as but not limited to IL-17C. In one embodiment, the IL-17RA-IL-17RE antagonist need not block the binding of IL-17C from binding to either IL-17RA or IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition.

Additional embodiments include methods of reducing pathogenic mediators released after IL-17RA-IL-17RE heteromeric receptor complex activation in cells expressing said complex in vivo using one or more of the IL-17RA-IL-17RE antagonists described herein. For example, a method of reducing pathogenic mediators released after IL-17RA-IL-17RE heteromeric receptor complex activation in cells expressing said complex in vivo comprises exposing said cells to an IL-17RA-IL-17RE antagonist, wherein the IL-17RA-IL-17RE antagonist binds IL-17RA and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RA. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition.

Additional embodiments include methods of reducing pathogenic mediators released after IL-17RA-IL-17RE heteromeric receptor complex activation in cells expressing said complex in vivo using one or more of the IL-17RA-IL-17RE antagonists described herein. For example, a method of reducing pathogenic mediators released after IL-17RA-IL-17RE heteromeric receptor complex activation in cells expressing said complex in vivo comprises exposing said cells to an IL-17RA-IL-17RE antagonist, wherein the IL-17RA-IL-17RE antagonist binds IL-17RE and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition.

Additional embodiments include methods of reducing pathogenic mediators released after IL-17RA-IL-17RE heteromeric receptor complex activation in cells expressing said complex in vivo using one or more of the IL-17RA-IL-17RE antagonists described herein. For example, a method of reducing pathogenic mediators released after IL-17RA-IL-17RE heteromeric receptor complex activation in cells expressing said complex in vivo comprises exposing said cells to an IL-17RA-IL-17RE antagonist, wherein the IL-17RA-IL-17RE antagonist binds both IL-17RA and IL-17RE and partially inhibit or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to either IL-17RA or IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition.

Additional embodiments comprise methods, as described above, wherein the pathogenic mediator is at least one of the following: IL-6, IL-8, G-CSF, GM-CSF, TNF-α, lipoclin-2, DEFB4, S100a8, and S100a9, as well as any other pathogenic mediator known in the art to be released from human cells expressing IL-17RA-IL-17RE heteromeric receptor complex and activated by IL-17C. Specific embodiments of IL-17RA-IL-17RE antagonists in the form of antibodies that can inhibit IL-17C from activating IL-17RA-IL-17RE receptor complex and thereby inhibit the pathogenic mediator are provided in Table 1.

Further embodiments include methods of treating IL-17 family member-associated disorders, such as but not limited to, inflammatory and autoimmune disorders with the IL-17RA-IL-17RE antagonists. Specific embodiments of IL-17RA-IL-17RE antagonists in the form of antibodies that can inhibit IL-17C from activating IL-17RA-IL-17RE receptor complex and thereby be used to treat such disorders are provided in Table 1.

Additional embodiments include methods of treating inflammation, wherein the IL-17RA-IL-17RE heteromeric receptor complex is partially or fully blocked from being activated by administering one or more of the IL-17RA-IL-17RE antagonists described herein. For example, a method of treating inflammation in a patient in need thereof comprises administering to said patient an IL-17RA-IL-17RE antagonist, wherein the IL-17RA-IL-17RE antagonist binds IL-17RA and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition. Specific embodiments include the antibodies provided in Table 1.

Additional embodiments include methods of treating inflammation, wherein the IL-17RA-IL-17RE heteromeric receptor complex is partially or fully blocked from being activated by administering one or more of the IL-17RA-IL-17RE antagonists described herein. For example, a method of treating inflammation in a patient in need thereof comprises administering to said patient an IL-17RA-IL-17RE antagonist, wherein the IL-17RA-IL-17RE antagonist binds IL-17RE and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition. Specific embodiments include the antibodies provided in Table 1.

Additional embodiments include methods of treating inflammation, wherein the IL-17RA-IL-17RE heteromeric receptor complex is partially or fully blocked from being activated by administering one or more of the IL-17RA-IL-17RE antagonists described herein. For example, a method of treating inflammation in a patient in need thereof comprises administering to said patient an IL-17RA-IL-17RE antagonist, wherein the IL-17RA-IL-17RE antagonist binds both IL-17RA and IL-17RE and partially inhibit or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition. Specific embodiments include the antibodies provided in Table 1.

Additional embodiments include methods of treating an autoimmune disorder, wherein the IL-17RA-IL-17RE heteromeric receptor complex is partially or fully blocked from being activated by administering one or more of the IL-17RA-IL-17RE antagonists described herein. For example, a method of treating an autoimmune disorder in a patient in need thereof comprises administering to said patient an IL-17RA-IL-17RE antagonist, wherein the IL-17RA-IL-17RE antagonist binds IL-17RA and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition. Specific embodiments include the antibodies provided in Table 1.

Additional embodiments include methods of treating an autoimmune disorder, wherein the IL-17RA-IL-17RE heteromeric receptor complex is partially or fully blocked from being activated by administering one or more of the IL-17RA-IL-17RE antagonists described herein. For example, a method of treating an autoimmune disorder in a patient in need thereof comprises administering to said patient an IL-17RA-IL-17RE antagonist, wherein the IL-17RA-IL-17RE antagonist binds IL-17RE and partially inhibits or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition. Specific embodiments include the antibodies provided in Table 1.

Additional embodiments include methods of treating an autoimmune disorder, wherein the IL-17RA-IL-17RE heteromeric receptor complex is partially or fully blocked from being activated by administering one or more of the IL-17RA-IL-17RE antagonists described herein. For example, a method of treating an autoimmune disorder in a patient in need thereof comprises administering to said patient an IL-17RA-IL-17RE antagonist, wherein the IL-17RA-IL-17RE antagonist binds both IL-17RA and IL-17RE and partially inhibit or fully inhibits association of IL-17RE with IL-17RA and thereby preventing IL-17RA-IL-17RE heteromeric receptor complex formation and activation through binding of IL-17 ligand family members, such as but not limited to IL-17C, and consequent release of pathogenic mediators. In one embodiment, the IL-17RA-IL-17RE antagonist need not block the binding of IL-17C from binding to IL-17RE. In alternative embodiments, the IL-17RA-IL-17RE antagonist may block the binding of IL-17C to either IL-17RA or IL-17RE. Additional embodiments comprise a method wherein said IL-17RA-IL-17RE antagonist is an antibody, as defined herein. Additional embodiments comprise a method wherein said IL-17RA-IL-17RE antagonist is an antibody, as defined herein, and the antibody is in the form of a pharmaceutical composition. Specific embodiments include the antibodies provided in Table 1.

Further embodiments include methods of treating inflammation and autoimmune disorders using IL-17RA-IL-17RE antagonists, as described above, and preferably the antibodies in Table 1, wherein the disorders include, but are not limited to, cartilage inflammation, and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, Graves' disease, Addison's disease, Raynaud's phenomenon, autoimmune hepatitis, GVHD, uveitis, cystic fibrosis, Beh9et's disease, pemphigus vulgaris, autoimmune hepatitis, heart failure, atherosclerosis, chronic urticaria, Type I diabetes, cancer, transplantation, and the like.

Further embodiments include methods of treating inflammation and autoimmune disorders by inhibiting IL-17A, IL-17B, IL-17C, IL-17D, IL-17E (IL-25), IL-17F, and IL-17A/F dimer activity using the antibodies in Table 1, wherein the disorders include, but are not limited to, cartilage inflammation, and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, Graves' disease, Addison's disease, Raynaud's phenomenon, autoimmune hepatitis, GVHD, uveitis, cystic fibrosis, Beh9et's disease, pemphigus vulgaris, autoimmune hepatitis, heart failure, atherosclerosis, chronic urticaria, Type I diabetes, cancer, transplantation, and the like.

Additional embodiments include pharmaceutical compositions comprising a therapeutically effective amount of one or more of an IL-17RA-IL-17RE antagonist together with a pharmaceutically acceptable diluent, carrier, solubilizer, emulsifier, preservative, and/or adjuvant. In addition, the invention provides methods of treating a patient by administering such pharmaceutical composition. Acceptable formulation materials are nontoxic to recipients at the dosages and concentrations employed. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the IL-17RA-IL-17RE antagonist. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute. In certain embodiments, IL-17RA-IL-17RE antagonist compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the IL-17RA-IL-17RE antagonist product may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the IL-17RA-IL-17RE antagonists may be provided in the form of a pyrogen-free, parenterally acceptable aqueous solution comprising the desired IL-17 receptor antigen binding protein in a pharmaceutically acceptable vehicle. A particularly suitable vehicle for parenteral injection is sterile distilled water in which the IL-17RA-IL-17RE antagonist is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Pharmaceutical compositions of the invention can be formulated for inhalation. In these embodiments, IL-17RA-IL17RE antagonist may be formulated as a dry, inhalable powder. Inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins.

It is also contemplated that formulations can be administered orally. IL-17RA-IL-17RE antagonists that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized Additional agents can be included to facilitate absorption of the IL-17RA-IL-17RE antagonist. Diluents, flavorings, low melting point waxes, vegetable oils, lubricants, suspending agents, tablet disintegrating agents, and binders may also be employed.

A pharmaceutical composition of the invention is preferably provided to comprise an effective quantity of one or more IL-17RA-IL-17RE antagonists in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving IL-17RA-IL-17RE antagonists in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 2:547-556), poly(2-hydroxyethyl-inethaciylate) (Langer, et al., 1981, J. Biomed. Mater. Res. 15:167-277 and Langer, 1982, Chem. Tech. 12:98-105), ethylene vinyl acetate (Langer, et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein, et al., 1985, Proc. Natl. Acad. Sci. U.S.A. 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The therapeutically effective amount of an IL-17RA-IL-17RE antagonist-containing pharmaceutical composition to be employed will depend, for example, upon the therapeutic context and objectives. One skilled in the art will appreciate that the appropriate dosage levels for treatment will vary depending, in part, upon the molecule delivered, the indication for which the IL-17RA-IL-17RE antagonist is being used, the route of administration, and the size (body weight, body surface or organ size) and/or condition (the age and general health) of the patient. In certain embodiments, the clinician may titer the dosage and modify the route of administration to obtain the optimal therapeutic effect. A typical dosage may range from about 0.1 μg/kg to up to about 30 mg/kg or more, depending on the factors mentioned above. In specific embodiments, the dosage may range from 0.1 mg/kg up to about 30 mg/kg, optionally from 1 mg/kg up to about 30 mg/kg or from 10 mg/kg up to about 5 mg/kg. Of course, it is understood that this is to be determined by qualified physicians and that these doses are merely exemplary. Dosing frequency will depend upon the pharmacokinetic parameters of the particular IL-17RA-IL-17RE antagonist in the formulation used. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In certain embodiments, the IL-17RA-IL-17RE antagonists can be administered to patients throughout an extended time period. Chronic administration of an IL-17RA-IL-17RE antagonist may minimize the adverse immune or allergic response commonly associated with IL-17RA-IL-17RE antagonist that are not fully human, for example an antibody raised against a human antigen in a non-human animal, for example, a non-fully human antibody or non-human antibody produced in a non-human species.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

The composition also may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

The IL-17RA-IL-17RE antagonists described herein may be used in combination (pretreatment, post-treatment, or concurrent treatment) with any of one or more TNF inhibitors for the treatment or prevention of the diseases and disorders recited herein, such as but not limited to, all forms of soluble TNF receptors including Etanercept (such as ENBREL®), as well as all forms of monomeric or multimeric p75 and/or p55 TNF receptor molecules and fragments thereof; anti-human TNF antibodies, such as but not limited to, Infliximab (such as REMICADE®), and D2E7 (such as HUMIRA®), and the like. Such TNF inhibitors include compounds and proteins which block in vivo synthesis or extracellular release of TNF. In a specific embodiment, the present invention is directed to the use of an IL-17RA-IL-17RE antagonist in combination (pre-treatment, post-treatment, or concurrent treatment) with any of one or more of the following TNF inhibitors: TNF binding proteins (soluble TNF receptor type-I and soluble TNF receptor type-II (“sTNFRs”), as defined herein), antiTNF antibodies, granulocyte colony stimulating factor; thalidomide; BN 50730; tenidap; E 5531; tiapafant PCA 4248; nimesulide; panavir; rolipram; RP 73401; peptide T; MDL 201,449A; (1R,3S)Cis-1-[9-(2,6-diaminopurinyl)]-3-hydroxy-4-cyclopentene hydrochloride; (1R,3R)-trans-1-(9-(2,6-diamino)purine]-3-acetoxycyclopentane; (1R,3R)-trans-1-[9-adenyl)-3-azidocyclopentane hydrochloride and (1R,3R)-trans-1-(6-hydroxy-purin-9-yl)-3-azidocyclo-pentane. TNF binding proteins are disclosed in the art (EP 308 378, EP 422 339, GB 2 218 101, EP 393 438, WO 90/13575, EP 398 327, EP 412 486, WO 91/03553, EP 418 014, JP 127,800/1991, EP 433 900, U.S. Pat. No. 5,136,021, GB 2 246 569, EP 464 533, WO 92/01002, WO 92/13095, WO 92/16221, EP 512 528, EP 526 905, WO 93/07863, EP 568 928, WO 93/21946, WO 93/19777, EP 417 563, WO 94/06476, and PCT International Application No. PCT/US97/12244). For example, EP 393 438 and EP 422 339 teach the amino acid and nucleic acid sequences of a soluble TNF receptor type I (also known as “sTNFR-I” or “30 kDa TNF inhibitor”) and a soluble TNF receptor type II (also known as “sTNFR-II” or “40 kDa TNF inhibitor”), collectively termed “sTNFRs”, as well as modified forms thereof (e.g., fragments, functional derivatives and variants). EP 393 438 and EP 422 339 also disclose methods for isolating the genes responsible for coding the inhibitors, cloning the gene in suitable vectors and cell types and expressing the gene to produce the inhibitors. Additionally, polyvalent forms (i.e., molecules comprising more than one active moiety) of sTNFR-I and sTNFR-II have also been disclosed. In one embodiment, the polyvalent form may be constructed by chemically coupling at least one TNF inhibitor and another moiety with any clinically acceptable linker, for example polyethylene glycol (WO 92/16221 and WO 95/34326), by a peptide linker (Neve et al. (1996), Cytokine, 8(5):365-370, by chemically coupling to biotin and then binding to avidin (WO 91/03553) and, finally, by combining chimeric antibody molecules (U.S. Pat. No. 5,116,964, WO 89/09622, WO 91/16437 and EP 315062. Anti-TNF antibodies include the MAK 195F Fab antibody (Holler et al. (1993), 1st International Symposium on Cytokines in Bone Marrow Transplantation, 147); CDP 571 anti-TNF monoclonal antibody (Rankin et al. (1995), British Journal of Rheumatology, 34:334-342); BAY X 1351 murine anti-tumor necrosis factor monoclonal antibody (Kieft et al. (1995), 7th European Congress of Clinical Microbiology and Infectious Diseases, page 9); CenTNF cA2 antiTNF monoclonal antibody (Elliott et al. (1994), Lancet, 344:1125-1127 and Elliott et al. (1994), Lancet, 344:1105-1110).

The IL-17RA-IL-17RE antagonists described herein may be used in combination with all forms of IL-1 inhibitors, such as but not limited to, kiniret (for example ANAKINRA®) (pretreatment, post-treatment, or concurrent treatment). Interleukin-1 receptor antagonist (IL-Ira) is a human protein that acts as a natural inhibitor of interleukin-1 Interleukin-1 receptor antagonists, as well as the methods of making and methods of using thereof, are described in U.S. Pat. No. 5,075,222; WO 91/08285; WO 91/17184; AU 9173636; WO 92/16221; WO 93/21946; WO 94/06457; WO 94/21275; FR 2706772; WO 94/21235; DE 4219626; WO 94/20517; WO 96/22793 and WO 97/28828. The proteins include glycosylated as well as non-glycosylated IL-1 receptor antagonists. Specifically, three preferred forms of IL-1ra (IL-1raα, IL-1raβ and IL-1rax), each being encoded by the same DNA coding sequence and variants thereof, are disclosed and described in U.S. Pat. No. 5,075,222. Methods for producing IL-1 inhibitors, particularly IL-1ras, are also disclosed in the U.S. Pat. No. 5,075,222 patent. An additional class of interleukin-I inhibitors includes compounds capable of specifically preventing activation of cellular receptors to IL-1. Such compounds include IL-1 binding proteins, such as soluble receptors and monoclonal antibodies. Such compounds also include monoclonal antibodies to the receptors. A further class of interleukin-1 inhibitors includes compounds and proteins that block in vivo synthesis and/or extracellular release of IL-1. Such compounds include agents that affect transcription of IL-1 genes or processing of IL-1 preproteins.

The IL-17RA-IL-17RE antagonists described herein may be used in combination with all forms of CD28 inhibitors, such as but not limited to, abatacept (for example ORENCIA®) (pretreatment, post-treatment, or concurrent treatment). The IL-17RA-IL-17RE antagonists may be used in combination with one or more cytokines, lymphokines, hematopoietic factor(s), and/or an anti-inflammatory agent (pretreatment, post-treatment, or concurrent treatment).

Treatment of the diseases and disorders recited herein can include the use of first line drugs for control of pain and inflammation in combination (pretreatment, post-treatment, or concurrent treatment) with treatment with one or more of the IL-17RA-IL-17RE antagonists provided herein. These drugs are classified as non-steroidal, anti-inflammatory drugs (NSAIDs). Secondary treatments include corticosteroids, slow acting antirheumatic dmgs (SAARDs), or disease modifying (DM) drugs. Information regarding the following compounds can be found in The Merck Manual of Diagnosis and Therapy, Sixteenth Edition, Merck, Sharp & Dohme Research Laboratories, Merck & Co., Rahway, N.J. (1992) and in Pharmaprojects, PJB Publications Ltd.

In a specific embodiment, the present invention is directed to the use of an IL-17RA-IL-17RE antagonist and any of one or more NSAIDs for the treatment of the diseases and disorders recited herein (pretreatment, post-treatment, or concurrent treatment). NSAIDs owe their anti-inflammatory action, at least in part, to the inhibition of prostaglandin synthesis (Goodman and Gilman in “The Pharmacological Basis of Therapeutics,” MacMillan 7th Edition (1985)). NSAIDs can be characterized into at least nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

In another specific embodiment, the present invention is directed to the use of an IL-17RA-IL-17RE antagonist in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate, magnesium salicylate, choline salicylate, diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide 0-acetic acid, salsalate, sodium salicylate and sulfasalazine. Structurally related salicylic acid derivatives having similar analgesic and anti-inflammatory properties are also intended to be encompassed by this group.

In an additional specific embodiment, the present invention is directed to the use of IL-17RA-IL-17RE antagonist in combination (pretreatment, post-treatment, or concurrent treatment) with any of one or more propionic acid derivatives, prodrug esters or pharmaceutically acceptable salts thereof. The propionic acid derivatives, prodrug esters, and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, naproxen sodium, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group.

In yet another specific embodiment, the present invention is directed to the use of an IL-17RA-IL-17RE antagonist in combination (pretreatment, post-treatment, or concurrent treatment) with any of one or more acetic acid derivatives, prodrug esters or pharmaceutically acceptable salts thereof. The acetic acid derivatives, prodrug esters, and pharmaceutically acceptable salts thereof comprise: acemetacin, alclofenac, amfenac, bufexamac, cinmetacin, clopirac, delmetacin, diclofenac potassium, diclofenac sodium, etodolac, felbinac, fenclofenac, fenclozic, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, tolmetin sodium, zidometacin and zomepirac. Structurally related acetic acid derivatives having similar analgesic and anti-inflammatory properties are also intended to be encompassed by this group. In another specific embodiment, the present invention is directed to the use of an IL-17RA-IL-17RE antagonist in combination (pretreatment, post-treatment, or concurrent treatment) with any of one or more fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefenamic acid, niflumic acid, talniflumate, terofenamate, tolfenamic acid and ufenamate Structurally related fenamic acid derivatives having similar analgesic and anti-inflammatory properties are also intended to be encompassed by this group.

In an additional specific embodiment, the present invention is directed to the use of an IL-17RA-IL-17RE antagonist in combination (pretreatment, post-treatment, or concurrent treatment) with any of one or more carboxylic acid derivatives, prodrug esters or pharmaceutically acceptable salts thereof. The carboxylic acid derivatives, prodrug esters, and pharmaceutically acceptable salts thereof which can be used comprise: clidanac, diflunisal, flufenisal, inoridine, ketorolac and tinoridine. Structurally related carboxylic acid derivatives having similar analgesic and anti-inflammatory properties are also intended to be encompassed by this group.

In yet another specific embodiment, the present invention is directed to the use of an IL-17RA-IL-17RE antagonist in combination (pretreatment, post-treatment, or concurrent treatment) with any of one or more butyric acid derivatives, prodrug esters or pharmaceutically acceptable salts thereof. The butyric acid derivatives, prodrug esters, and pharmaceutically acceptable salts thereof comprise: bumadizon, butibufen, fenbufen and xenbucin. Structurally related butyric acid derivatives having similar analgesic and anti-inflammatory properties are also intended to be encompassed by this group.

In another specific embodiment, the present invention is directed to the use of an IL-17RA-IL-17RE antagonist in combination (pretreatment, post-treatment, or concurrent treatment) with any of one or more oxicams, prodrug esters, or pharmaceutically acceptable salts thereof. The oxicams, prodrug esters, and pharmaceutically acceptable salts thereof comprise: droxicam, enolicam, isoxicam, piroxicam, sudoxicam, tenoxicam and 4-hydroxyl-1,2-benzothiazine 1,1-dioxide 4-(Nphenyl)-carboxamide. Structurally related oxicams having similar analgesic and anti-inflammatory properties are also intended to be encompassed by this group.

In still another specific embodiment, the present invention is directed to the use of an IL-17RA-IL-17RE antagonist in combination (pretreatment, post-treatment, or concurrent treatment) with any of one or more pyrazoles, prodrug esters, or pharmaceutically acceptable salts thereof. The pyrazoles, prodrug esters, and pharmaceutically acceptable salts thereof which may be used comprise: difenamizole and epirizole. Structurally related pyrazoles having similar analgesic and anti-inflammatory properties are also intended to be encompassed by this group.

In an additional specific embodiment, the present invention is directed to the use of an IL-17RA-IL-17RE antagonist in combination (pretreatment, post-treatment or, concurrent treatment) with any of one or more pyrazolones, prodrug esters, or pharmaceutically acceptable salts thereof. The pyrazolones, prodrug esters and pharmaceutically acceptable salts thereof which may be used comprise: apazone, azapropazone, benzpiperylon, feprazone, mofebutazone, morazone, oxyphenbutazone, phenylbutazone, pipebuzone, propylphenazone, ramifenazone, suxibuzone and thiazolinobutazone. Structurally related pyrazalones having similar analgesic and anti-inflammatory properties are also intended to be encompassed by this group.

In another specific embodiment, the present invention is directed to the use of an IL-17RA-IL-17RE antagonist in combination (pretreatment, post-treatment, or concurrent treatment) with any of one or more of the following NSAIDs: s-acetamidocaproic acid, S-adenosyl-methionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopilioline, fosfosal, guaimesal, guaiazolene, isonixirn, lefetamine HCl, leflunomide, lofemizole, lotifazole, lysin clonixinate, meseclazone, nabumetone, nictindole, nimesulide, orgotein, orpanoxin, oxaceprol, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, AI77B, AP504, AU8001, BPPC, BW540C, CHINOIN 127, CNIOO, EB382, EL508, F1044, FK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770. Structurally related NSAIDs having similar analgesic and anti-inflammatory properties to the NSAIDs are also intended to be encompassed by this group.

In still another specific embodiment, the present invention is directed to the use of an IL-17RA-IL-17RE antagonist in combination (pretreatment, post-treatment or concurrent treatment) with any of one or more corticosteroids, prodrug esters or pharmaceutically acceptable salts thereof for the treatment of the diseases and disorders recited herein. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flucinolone acetonide, flunisolide, fluocinonide, fluorocinolone acetonide, fluocortin butyl, fluocortolone, fluocortolone hexanoate, diflucortolone valerate, fluorometholone, fluperolone acetate, fluprednidene acetate, fluprednisolone, flurandenolide, formocortal, halcinonide, halometasone, halopredone acetate, hydro-cortamate, hydrocortisone, hydrocortisone acetate, hydro-cortisone butyrate, hydrocortisone phosphate, hydrocortisone 21-sodium succinate, hydrocortisone tebutate, mazipredone, medrysone, meprednisone, methylprednisolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

In another specific embodiment, the present invention is directed to the use of an IL-17RA-IL-17RE antagonist in combination (pretreatment, post-treatment, or concurrent treatment) with any of one or more slow-acting antirheumatic drugs (SAARDs) or disease modifying antirheumatic drugs (DMARDS), prodrug esters, or pharmaceutically acceptable salts thereof for the treatment of the diseases and disorders recited herein. SAARDs or DMARDS, prodrug esters and pharmaceutically acceptable salts thereof comprise: allocupreide sodium, auranofin, aurothioglucose, aurothioglycanide, azathioprine, brequinar sodium, bucillamine, calcium 3-aurothio-2-propanol-1-sulfonate, chlorambucil, chloroquine, clobuzarit, cuproxoline, cyclo-phosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxychloroquine sulfate, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine. Structurally related SAARDs or DMARDs having similar analgesic and anti-inflammatory properties are also intended to be encompassed by this group.

In another specific embodiment, the present invention is directed to the use of an IL-17RA-IL-17RE antagonist (pretreatment, post-treatment, or concurrent treatment) with any of one or more COX2 inhibitors, prodmg esters or pharmaceutically acceptable salts thereof for the treatment of the diseases and disorders recited herein. Examples of COX2 inhibitors, prodrug esters or pharmaceutically acceptable salts thereof include, for example, celecoxib. Structurally related COX2 inhibitors having similar analgesic and anti-inflammatory properties are also intended to be encompassed by this group. Examples of COX-2 selective inhibitors include but not limited to etoricoxib, valdecoxib, celecoxib, licofelone, lumiracoxib, rofecoxib, and the like.

In still another specific embodiment, the present invention is directed to the use of an IL-17RA-IL-17RE antagonist in combination (pretreatment, post-treatment, or concurrent treatment) with any of one or more antimicrobials, prodrug esters or pharmaceutically acceptable salts thereof for the treatment of the diseases and disorders recited herein Antimicrobials include, for example, the broad classes of penicillins, cephalosporins and other beta-lactams, aminoglycosides, azoles, quinolones, macrolides, rifamycins, tetracyclines, sulfonamides, lincosamides and polymyxins. The penicillins include, but are not limited to penicillin G, penicillin V, methicillin, nafcillin, oxacillin, cloxacillin, dicloxacillin, floxacillin, ampicillin, ampicillin/sulbactam, amoxicillin, amoxicillin/clavulanate, hetacillin, cyclacillin, bacampicillin, carbenicillin, carbenicillin indanyl, ticarcillin, ticarcillin/clavulanate, azlocillin, mezlocillin, peperacillin, and mecillinam. The cephalosporins and other beta-lactams include, but are not limited to cephalothin, cephapirin, cephalexin, cephradine, cefazolin, cefadroxil, cefaclor, cefamandole, cefotetan, cefoxitin, ceruroxime, cefonicid, ceforadine, cefixime, cefotaxime, moxalactam, ceftizoxime, cetriaxone, cephoperazone, ceftazidime, imipenem and aztreonam. The aminoglycosides include, but are not limited to streptomycin, gentamicin, tobramycin, amikacin, netilmicin, kanamycin and neomycin. The azoles include, but are not limited to fluconazole. The quinolones include, but are not limited to nalidixic acid, norfloxacin, enoxacin, ciprofloxacin, ofloxacin, sparfloxacin and temafloxacin. The macrolides include, but are not limited to erythomycin, spiramycin and azithromycin. The rifamycins include, but are not limited to rifampin. The tetracyclines include, but are not limited to spicycline, chlortetracycline, clomocycline, demeclocycline, deoxycycline, guamecycline, lymecycline, meclocycline, methacycline, minocycline, oxytetracycline, penimepicycline, pipacycline, rolitetracycline, sancycline, senociclin and tetracycline. The sulfonamides include, but are not limited to sulfanilamide, sulfamethoxazole, sulfacetamide, sulfadiazine, sulfisoxazole and co-trimoxazole (trimethoprim/sulfamethoxazole). The lincosamides include, but are not limited to clindamycin and lincomycin. The polymyxins (polypeptides) include, but are not limited to polymyxin B and colistin.

### 4.0 Screening Assays

Additional embodiments include methods of screening for antagonists of the IL-17RA-IL-17RE heteromeric receptor complex. Screening assay formats that are known in the art and are adaptable to identifying antagonists of the IL-17RA-IL-17RE heteromeric receptor complex are contemplated. For example: a method of screening for an antagonist of an IL-17RA-IL-17RE heteromeric receptor complex, comprising providing an IL-17RA and an IL-17RE in an IL-17RA-IL-17RE heteromeric receptor complex; exposing the IL-17RA-IL-17RE heteromeric receptor complex to IL-17C; exposing a candidate agent to said receptor complex in the presence of IL-17C; and determining the amount of receptor complex formation relative to not having been exposed to the candidate agent. The step of exposing a candidate agent to the receptor complex may be before, during, or after IL-17RA and IL-17RE form an IL-17RA-IL-17RE heteromeric receptor complex.

Additional embodiments include a method of screening for an antagonist of IL-17RA-IL-17RE heteromeric receptor complex activation, comprising providing an IL-17RA and an IL-17RE in an IL-17RA-IL-17RE heteromeric receptor complex; exposing a candidate agent to said receptor complex; adding one or more IL-17 ligands, preferably IL-17C; and determining the amount of IL-17RA-IL-17RE heteromeric receptor complex activation relative to not having been exposed to the candidate agent. Candidate agents that decrease IL-17RA-IL-17RE heteromeric receptor complex activation in the presence of one or more IL-17 ligands, preferably IL-17C, as measured by a biologically relevant readout (see below), are considered positive. The IL-17 ligand may be IL-17C or any other IL-17 ligand that binds and activates the IL-17RA-IL-17RE heteromeric receptor complex. Activation is defined elsewhere in the specification. Relevant biological readouts include IL-6, IL-8, G-CSF, GM-CSF, TNFα, lipoclin-2, DEFB4, S100a8, and S100a9, as well as any other molecule known in the art to be released from any cells expressing IL-17RA-IL-17RE heteromeric receptor complex. The step of exposing a candidate agent to the receptor complex may be before, during, or after IL-17RA and IL-17RE form an IL-17RA-IL-17RE heteromeric receptor complex. It is understood that a candidate agent may partially inhibit IL-17RA-IL-17RE heteromeric receptor complex, i.e., less than 100% inhibition. Under certain assay conditions a candidate agent may completely inhibit IL-17RA-IL-17RE heteromeric receptor complex.

In one aspect, the invention provides for cell-based assays to detect the effect of candidate agents on the association of IL-17RA and IL-17RE, the IL-17RA-IL-17RE heteromeric receptor complex, as well as activation of the IL-17RA-IL-17RE heteromeric receptor complex. Thus the invention provides for the addition of candidate agents to cells to screen for IL-17RA-IL-17RE heteromeric receptor complex antagonists.

By “candidate agent” or “candidate drug” as used herein describes any molecule, such as but not limited to peptides, fusion proteins of peptides (e.g., peptides that bind IL-17RA, IL-17RE, or the IL-17RA-IL-17RE heteromeric receptor complex that are covalently or non-covalently bound to other proteins, such as fragments of antibodies or protein-based scaffolds known in the art), proteins, antibodies, small organic molecules including known drugs and drug candidates, polysaccharides, fatty acids, vaccines, nucleic acids, etc. that can be screened for activity as outlined herein. Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In alternative embodiments, the candidate bioactive agents may be proteins or fragments of proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In some embodiments, the candidate agents are peptides. In this embodiment, it can be useful to use peptide constructs that include a presentation structure. By “presentation structure” or grammatical equivalents herein is meant a sequence, which, when fused to candidate bioactive agents, causes the candidate agents to assume a conformationally restricted form. Proteins interact with each other largely through conformationally constrained domains Although small peptides with freely rotating amino and carboxyl termini can have potent functions as is known in the art, the conversion of such peptide structures into pharmacologic agents is difficult due to the inability to predict sidechain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures. Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc. See U.S. Pat. No. 6,153,380, incorporated by reference.

Of particular use in screening assays are phage display libraries; see e.g., U.S. Pat. Nos. 5,223,409; 5,403,484; 5,571,698; and 5,837,500, all of which are expressly incorporated by reference in their entirety for phage display methods and constructs. In general, phage display libraries can utilize synthetic protein (e.g. peptide) inserts, or can utilize genomic, cDNA, etc. digests.

Depending on the assay and desired outcome, a wide variety of cell types may be used, including eukaryotic and prokaryotic cells, with mammalian cells, and human cells, finding particular use in the invention. In one embodiment, the cells may be genetically engineered, for example they may contain exogenous nucleic acids, such as those encoding IL-17RA and IL-17RC. In some instances, the IL-17RA and IL-17RC proteins of the invention are engineered to include labels such as epitope tags, such as but not limited to those for use in immunoprecipitation assays or for other uses.

The candidate agents are added to the cells and allowed to incubate for a suitable period of time. The step of exposing a candidate agent to the receptor complex may be before, during, or after IL-17RA and IL-17RE form an IL-17RA-IL-17RE heteromelic receptor complex. In one embodiment, the association of IL-17RA and IL-17RE is evaluated in the presence and absence of the candidate agents. For example, by using tagged constructs and antibodies, immunoprecipitation experiments can be done. Candidate agents that interfere with IL-17RA and IL-17RE association are then tested for IL-17 ligand family member, preferably IL-17C signaling activity, such as by testing for expression of genes that are activated by the IL-17 ligand family member, as mentioned above.

In some embodiments, the IL-17RA and/or IL-17RE proteins are fusion proteins. For example, receptor proteins may be modified in a way to form chimeric molecules comprising an apoprotein fused to another, heterologous polypeptide or amino acid sequence. In one embodiment, such a chimeric molecule comprises a fusion of a receptor with a tag polypeptide which provides an epitope to which an anti-tag antibody can selectively bind. The epitope tag is generally placed at the amino- or carboxyl-terminus of the receptor protein. The presence of such epitope-tagged forms of the receptor can be detected using an antibody against the tag polypeptide. Also, provision of the epitope tag enables the receptor polypeptide to be readily purified by affinity purification using an anti-tag antibody or another type of affinity matrix that binds to the epitope tag. These epitope tags can be used for immobilization to a solid support, as outlined herein.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the FLAGG™-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

### 5.0 Making Antigen Binding Proteins

Suitable host cells for expression of ILIL-17RA-IL-17RE antagonists, specifically antigen binding protein and preferably the antibodies in Table 1, include prokaryotes, yeast, or higher eukaryotic cells. Appropriate cloning and expression vectors for use with bacterial, fungal, yeast, and mammalian cellular hosts are described, for example, in Pouwels et al. Cloning Vectors: A Laboratory; Manual, Elsevier, New York, (1985). Cell-free translation systems could also be employed to produce LDCAM polypeptides using RNAs derived from DNA constructs disclosed herein.

Prokaryotes include gram negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, an IL-17RA-IL-17RE heteromeric receptor complex antigen binding protein may include an N-terminal methionine residue to facilitate expression of the recombinant polypeptide in the prokaryotic host cell. The N-terminal Met may be cleaved from the expressed recombinant IL-17RA-IL-17RE heteromeric receptor complex antigen binding protein.

IL-17RA-IL-17RE heteromeric receptor complex antigen binding proteins may be expressed in yeast host cells, preferably from the Saccharomyces genus (e.g., S. cerevisiae). Other genera of yeast, such as Pichia, K. lactis or Kluyveromyces, may also be employed. Yeast vectors will often contain an origin of replication sequence from a yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase Other suitable vectors and promoters for use in yeast expression are further described in Hitzeman, EPA-73,657 or in Fleer et. al., Gene, 107:285-195 (1991); and van den Berg et. al., Bio/Technology, 8:135-139 (1990). Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli may be constructed by inserting DNA sequences from pBR322 for selection and replication in E. coli (Ampr gene and origin of replication) into the above-described yeast vectors.

The yeast α-factor leader sequence may be employed to direct secretion of the IL-17RA-IL-17RE heteromeric receptor complex antigen binding protein. The α-factor leader sequence is often inserted between the promoter sequence and the structural gene sequence. See, e.g., Kurjan et al., Cell 30:933, 1982; Bitter et al., Proc. Natl. Acad. Sci. USA 81:5330, 1984; U.S. Pat. No. 4,546,082; and EP 324,274. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp+ transformants in a selective medium, wherein the selective medium consists of 0.67% yeast nitrogen base, 0.5% casamino acids, 2% glucose, 10 ug/ml adenine and 20 ug/ml uracil. Yeast host cells transformed by vectors containing ADH2 promoter sequence may be grown for inducing expression in a “rich” medium. An example of a rich medium is one consisting of 1% yeast extract, 2% peptone, and 1% glucose supplemented with 80 ug/ml adenine and 80 ug/ml uracil. Derepression of the ADH2 promoter occurs when glucose is exhausted from the medium.

Mammalian or insect host cell culture systems could also be employed to express recombinant IL-17RA-IL-17RE heteromeric receptor complex antigen binding proteins. Baculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line derived from the African green monkey kidney cell line CVI (ATCC CCL 70) as described by McMahan et al. (EMBO J. 10: 2821, 1991).

Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide other genetic elements for expression of a structural gene sequence in a mammalian host cell. Viral early and late promoters are particularly useful because both are easily obtained from a viral genome as a fragment which may also contain a viral origin of replication (Fiers et al., Nature 273:113, 1978). Smaller or larger SV40 fragments may also be used, provided the approximately 250 bp sequence extending from the Hind III site toward the Bgl I site located in the SV 40 viral origin of replication site is included.

Exemplary expression vectors for use in mammalian host cells can be constructed as disclosed by Okayama and Berg (Mol. Cell. Biol. 3:280, 1983). A useful system for stable high level expression of mammalian cDNAs in C127 murine mammaiy epithelial cells can be constructed substantially as described by Cosman et al. (Mol. Immunol. 23:935, 1986). A useful high expression vector, PMLSV N1/N4, described by Cosman et al., Nature 312:768, 1984 has been deposited as ATCC 39890. Additional useful mammalian expression vectors are described in EP-A-0367566, and in U.S. patent application Ser. No. 07/701,415, filed May 16, 1991, incorporated by reference herein. The vectors may be derived from retroviruses. In place of the native signal sequence, and in addition to an initiator methionine, a heterologous signal sequence may be added, such as the signal sequence for IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type II IL-1 receptor signal peptide described in EP 460,846.

IL-17RA-IL-17RE heteromeric receptor complex antigen binding proteins, as an isolated, purified or homogeneous protein according to the invention, may be produced by recombinant expression systems as described above or purified from naturally occurring cells. IL-17RA-IL-17RE heteromeric receptor complex antigen binding proteins can be purified to substantial homogeneity, as indicated by a single protein band upon analysis by SDS-polyacrylamide gel electrophoresis (SD S-PAGE).

One process for producing IL-17RA-IL-17RE heteromeric receptor complex antigen binding proteins comprises culturing a host cell transformed with an expression vector comprising a DNA sequence that encodes at least one IL-17RA-IL-17RE heteromeric receptor complex antigen binding protein under conditions sufficient to promote expression of said IL-17RA-IL-17RE heteromeric receptor complex antigen binding protein. IL-17RA-IL-17RE heteromeric receptor complex antigen binding protein is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. For example, when expression systems that secrete the recombinant protein are employed, the culture medium first may be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred Finally, one or more reversed-phase high performance liquid chromatography (RP-HPLC) steps employing hydrophobic RP-HPLC media, (e.g., silica gel having pendant methyl or other aliphatic groups) can be employed to further purify IL-17RA-IL-17RE heteromeric receptor complex antigen binding proteins. Some or all of the foregoing purification steps, in various combinations, are well known and can be employed to provide a substantially homogeneous recombinant protein.

It is possible to utilize an affinity column comprising the IL-17RA, or IL-17RC, or both IL-17RA and IL-17RC, or a IL-17RA-IL-17RE heteromeric receptor complex proteins to affinity-purify expressed IL-17RA-IL-17RE heteromeric receptor complex antigen binding proteins. IL-17RA-IL-17RE heteromeric receptor complex antigen binding proteins can be removed from an affinity column using conventional techniques, e.g., in a high salt elution buffer and then dialyzed into a lower salt buffer for use or by changing pH or other components depending on the affinity matrix utilized. Alternatively, the affinity column may comprise an antibody that binds IL-17RA-IL-17RE heteromeric receptor complex antigen binding proteins.

Recombinant protein produced in bacterial culture can be isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps Finally, RP-HPLC can be employed for final purification steps. Microbial cells can be disrupted by any convenient method, including freeze-thaw cycling, sonication, mechanical disruption, or use of cell lysing agents.

Transformed yeast host cells may be employed to express IL-17RA-IL-17RE heteromeric receptor complex antigen binding proteins as a secreted polypeptide in order to simplify purification. Secreted recombinant polypeptide from a yeast host cell fermentation can be purified by methods analogous to those disclosed by Urdal et al. 1984, J. Chromatog. 296: 171. Urdal et al. describe two sequential, reversed-phase HPLC steps for purification of recombinant human IL-2 on a preparative HPLC column.

Standard techniques may be used for recombinant DNA, oligonucleotide synthesis, tissue culture and transformation, protein purification etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manual, 3rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytical chemistry, organic chemistry, and medicinal and pharmaceutical chemistry described herein are those well known and commonly used in the art. Standard techniques may be used for chemical synthesis, chemical analyses, pharmaceutical preparation, formulation, and delivery and treatment of patients.

### Examples

### 1. Evaluation of the IL-17 Pathway in a Psoriasis Model

### A. Histological Analysis of the TPA (12-O-Tetradecanoylphorbol-13-Acetate) Induced IL-36α Transgenic Mouse Skin Inflammation Model

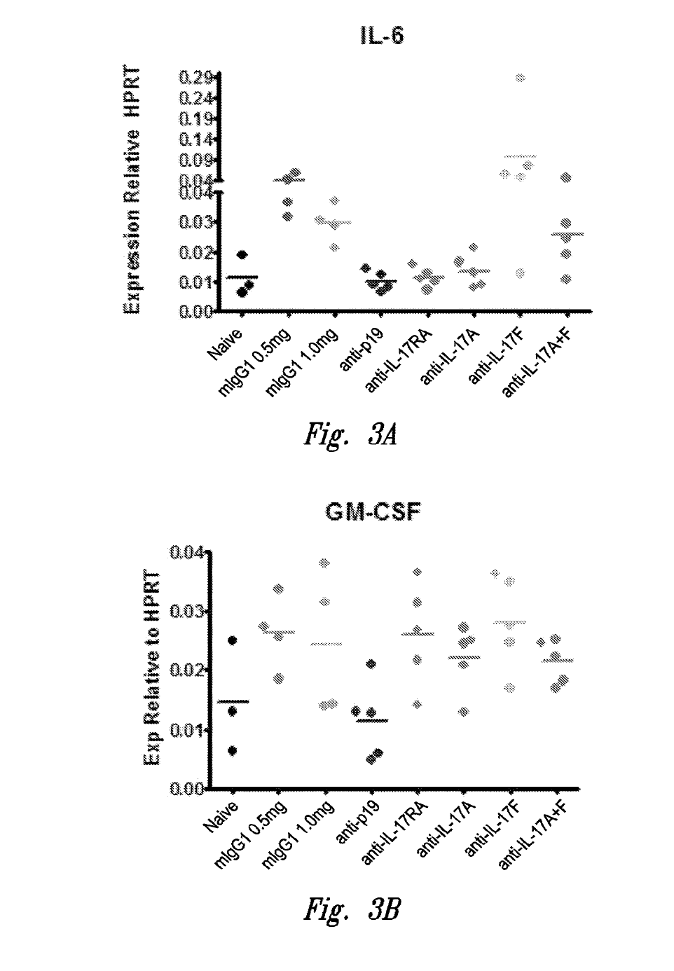

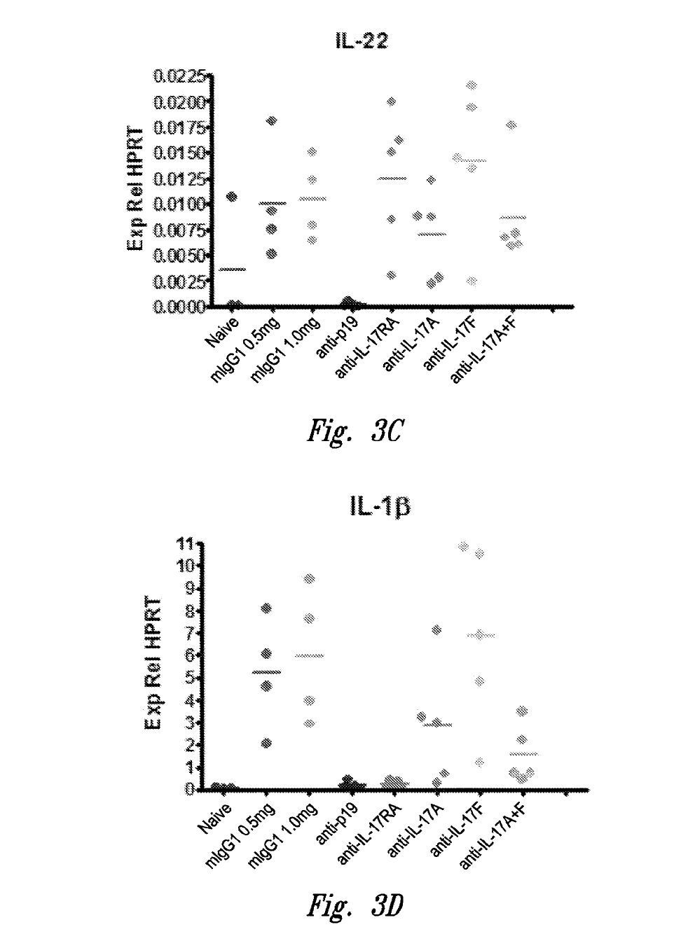

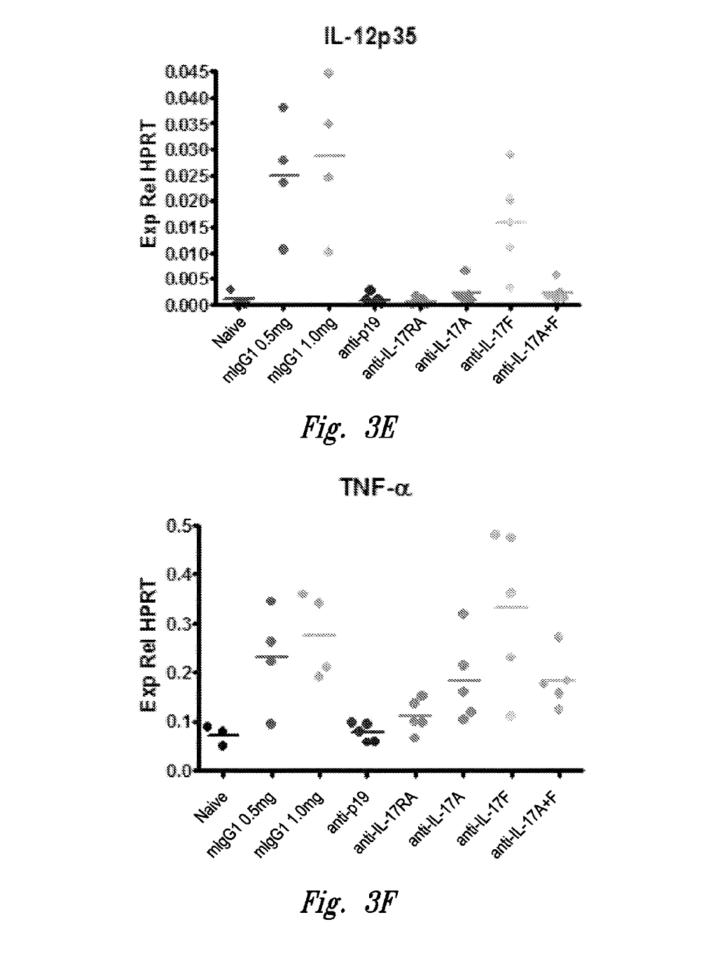

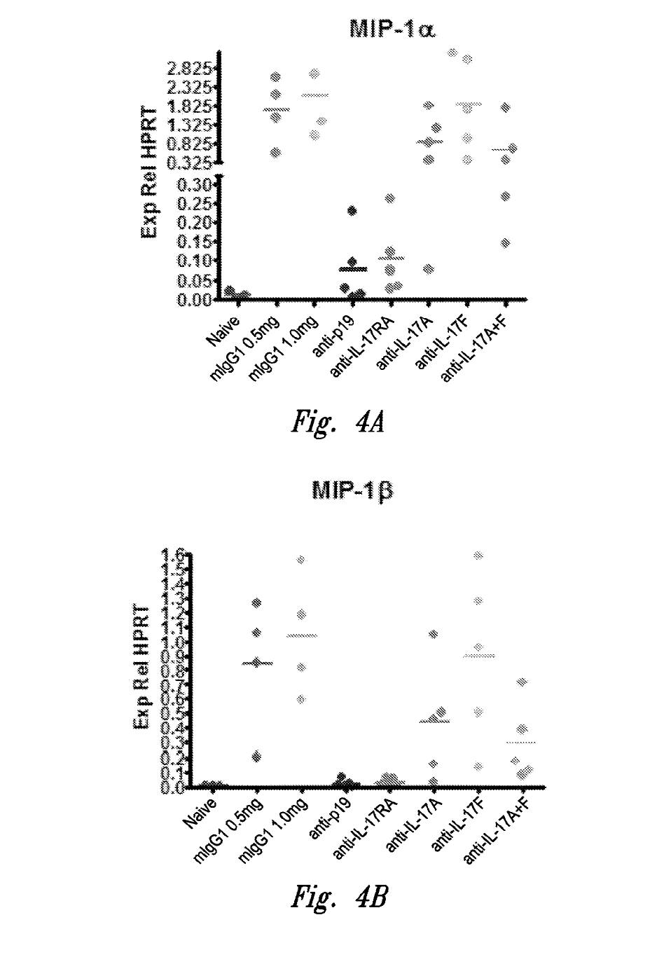

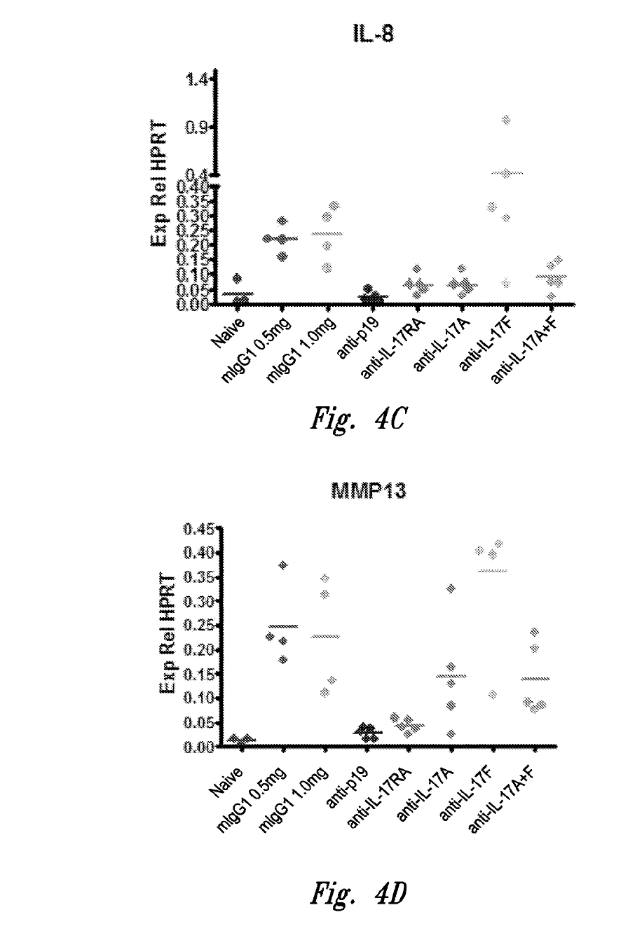

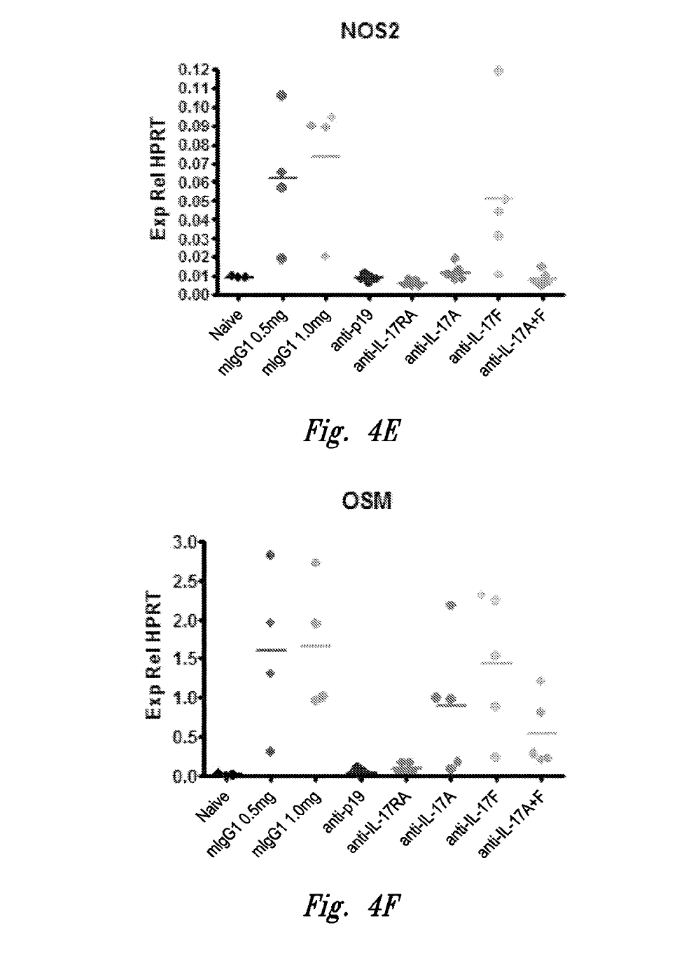

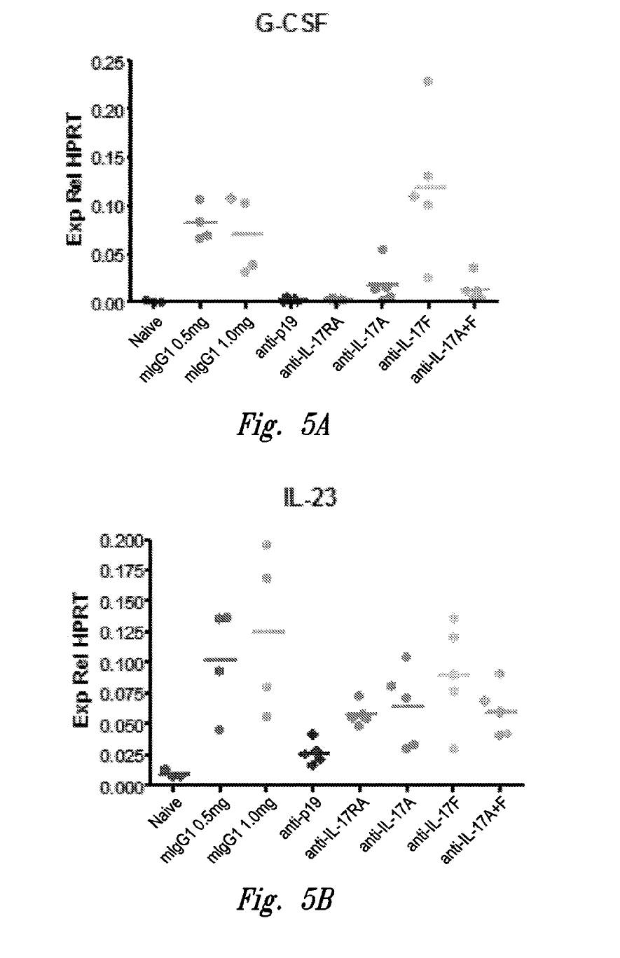

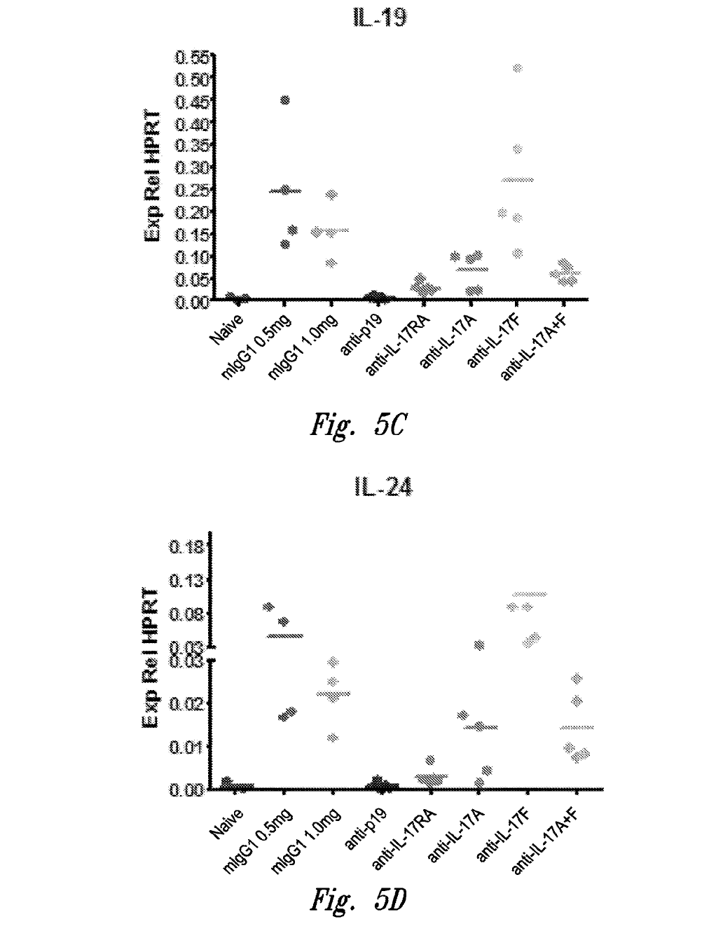

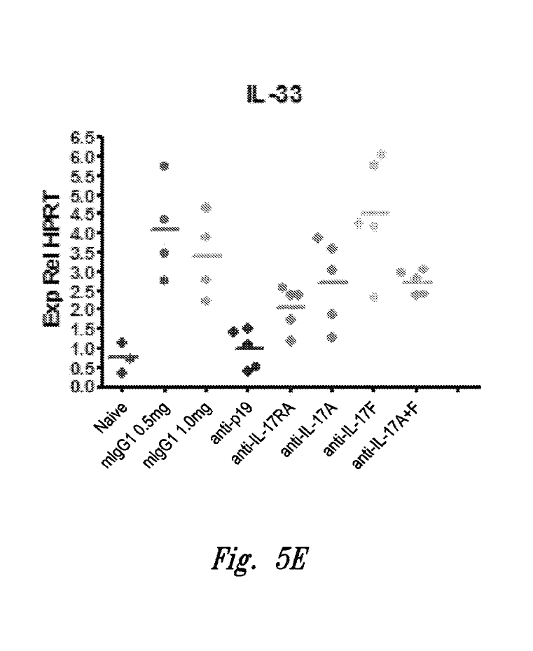

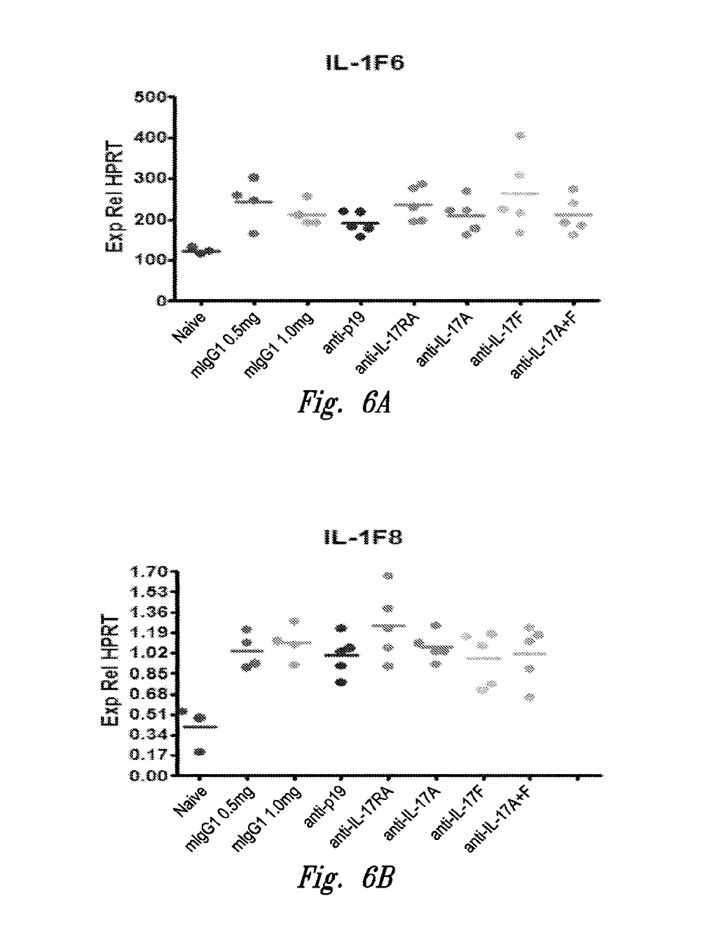

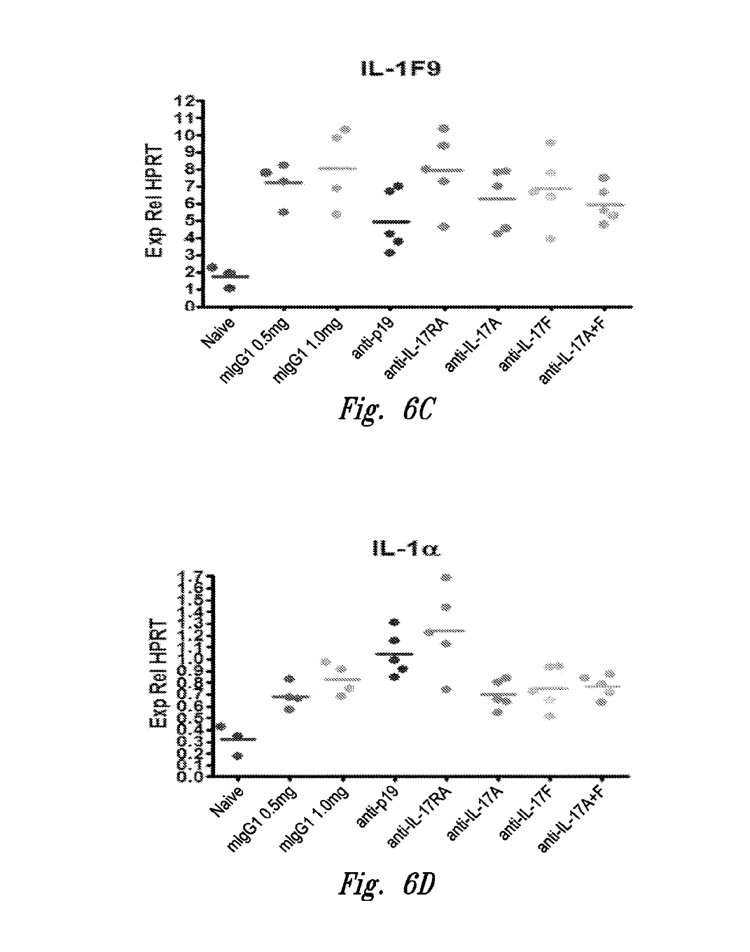

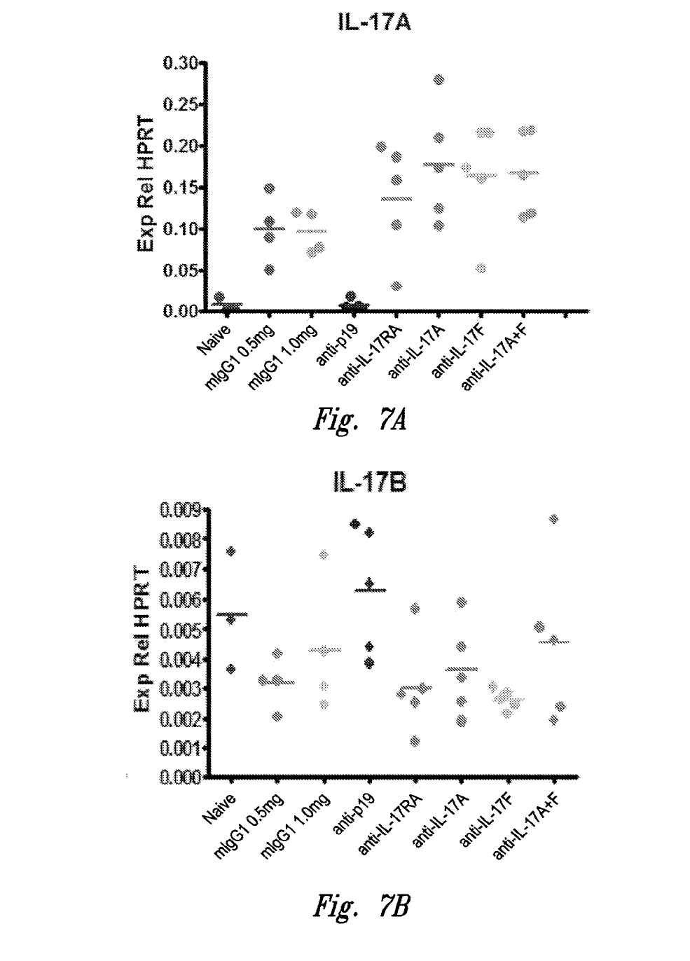

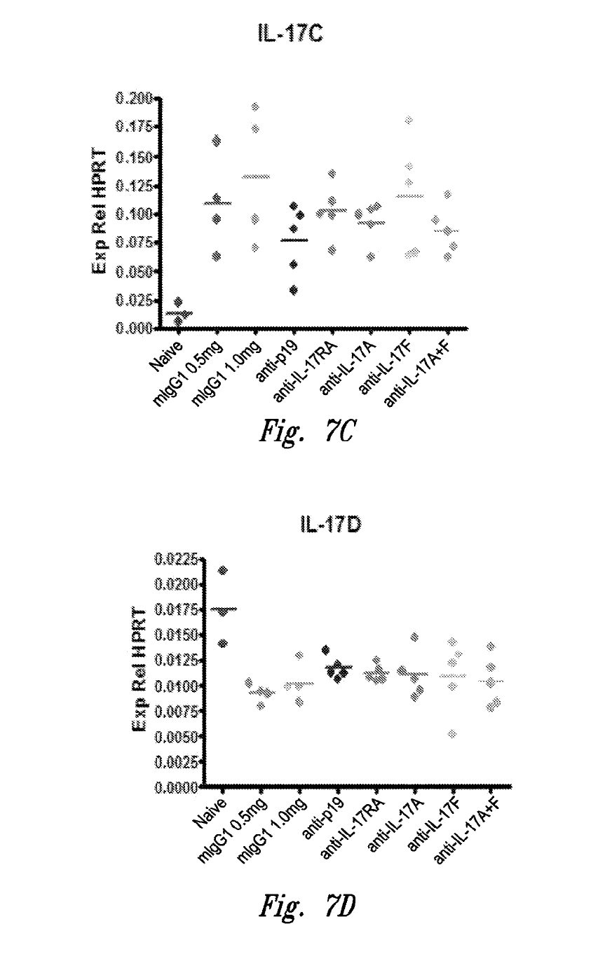

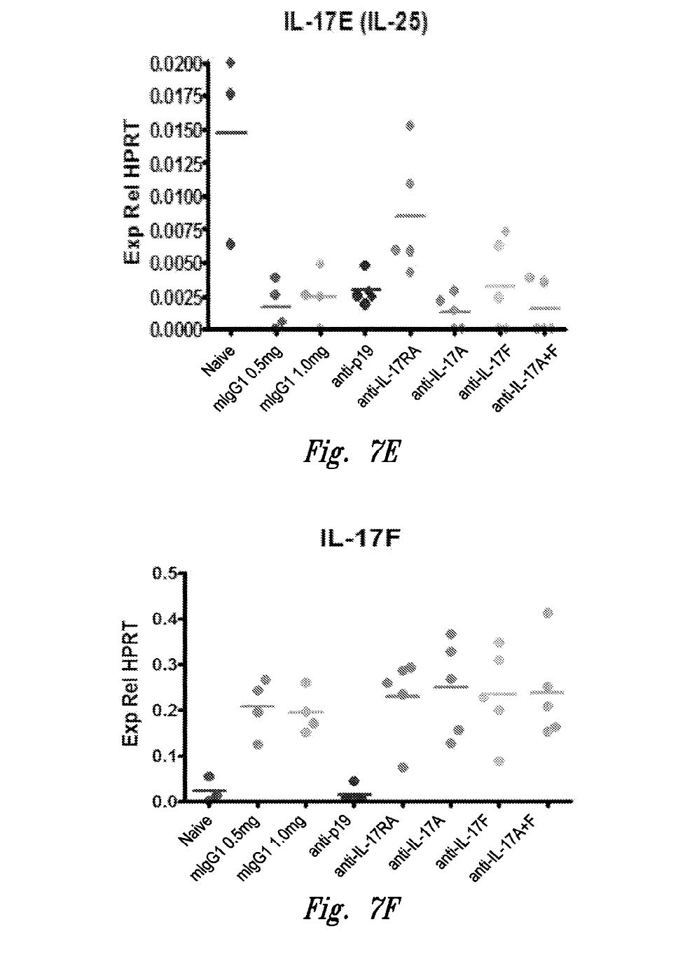

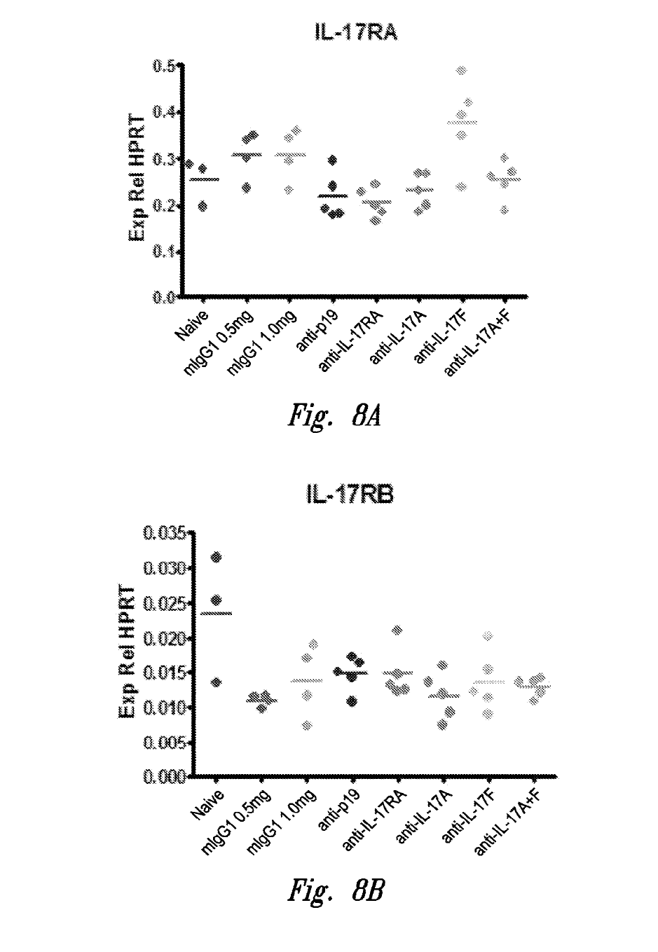

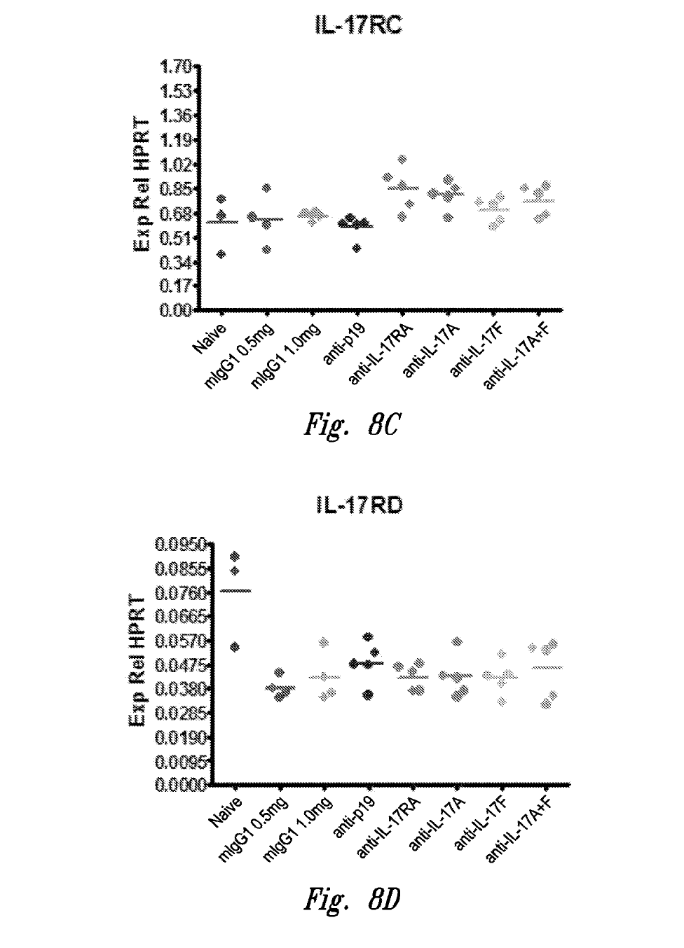

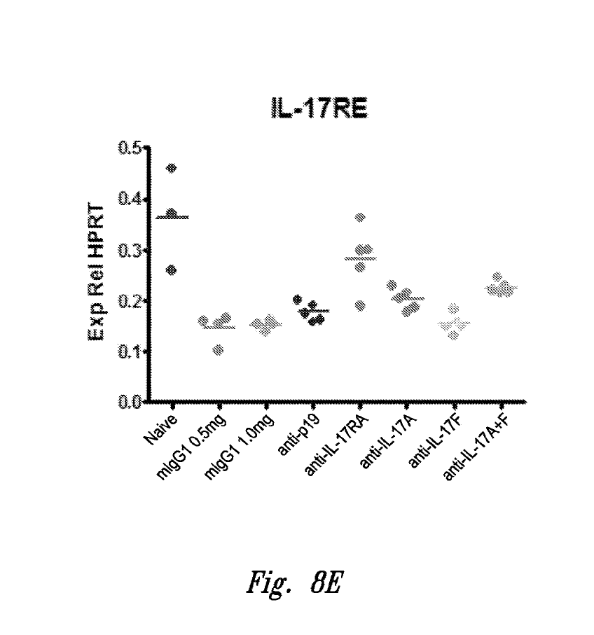

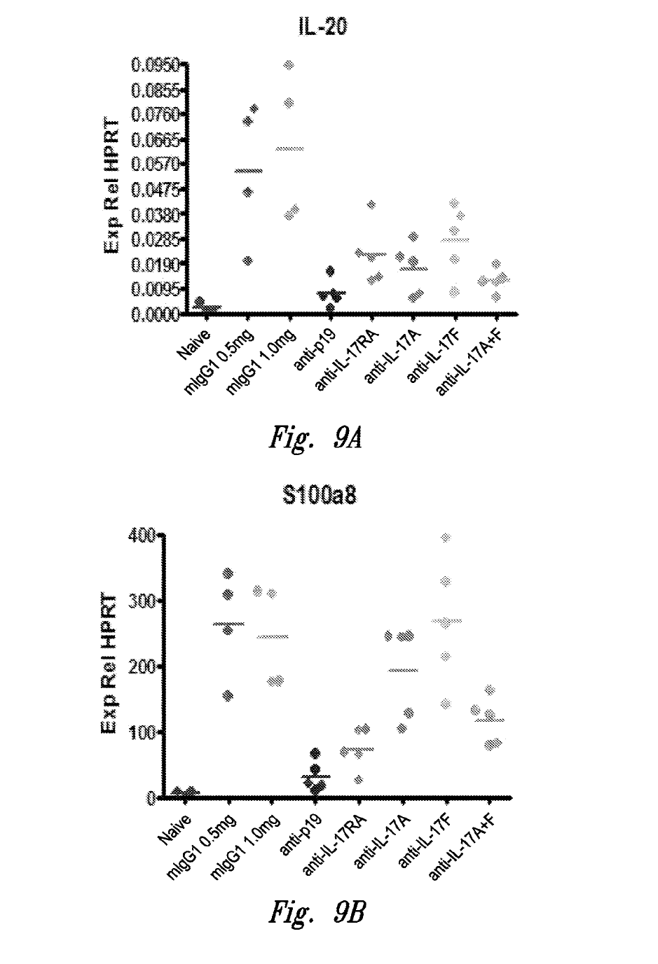

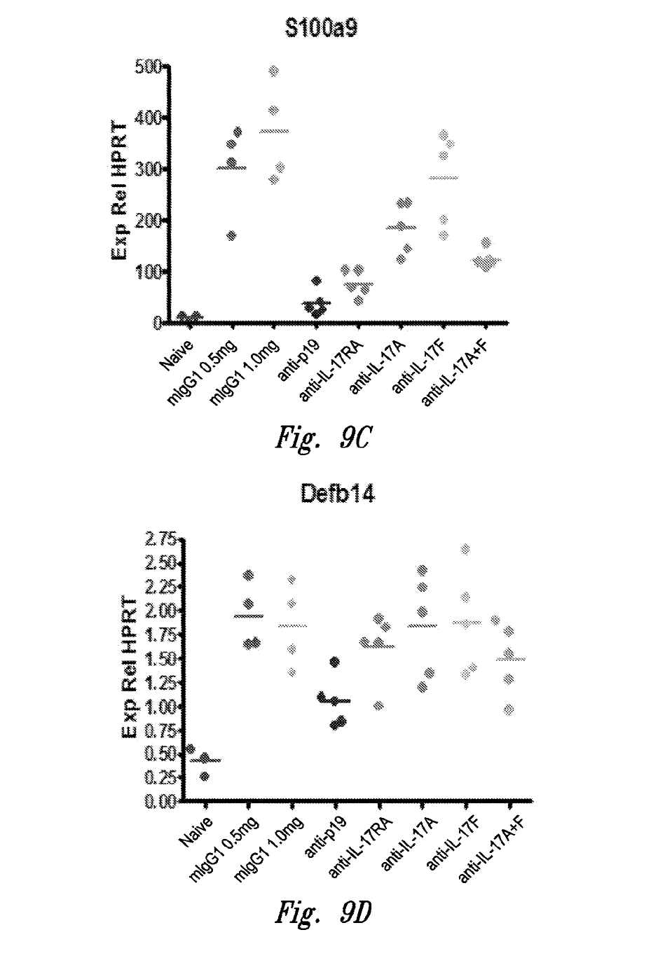

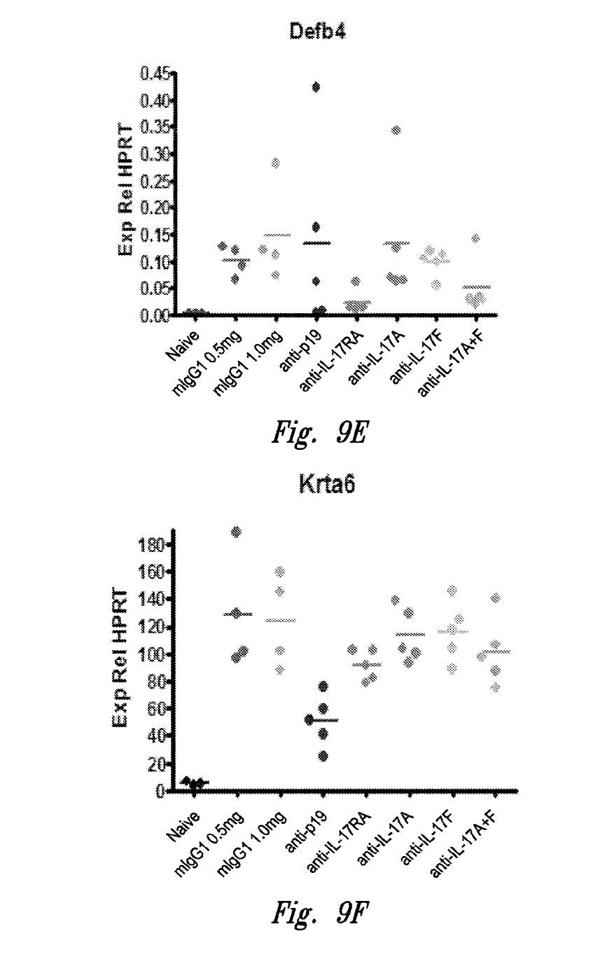

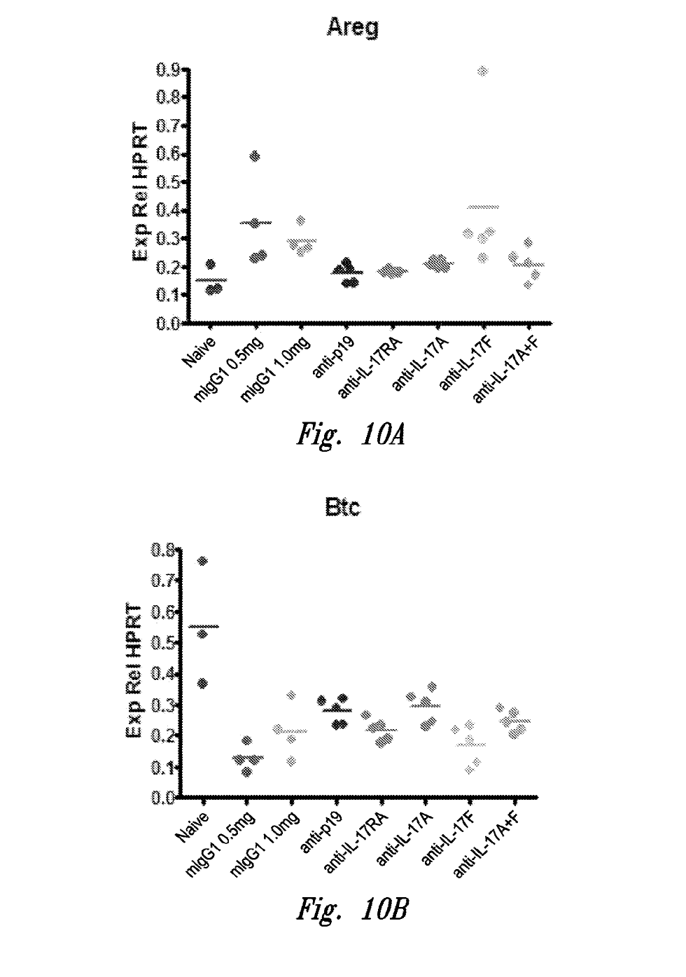

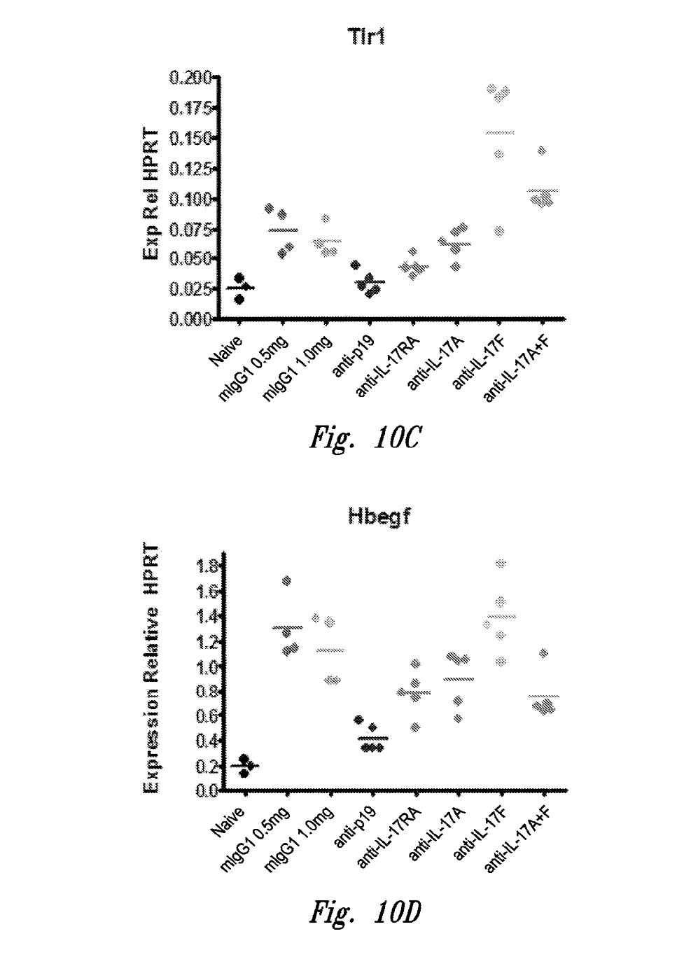

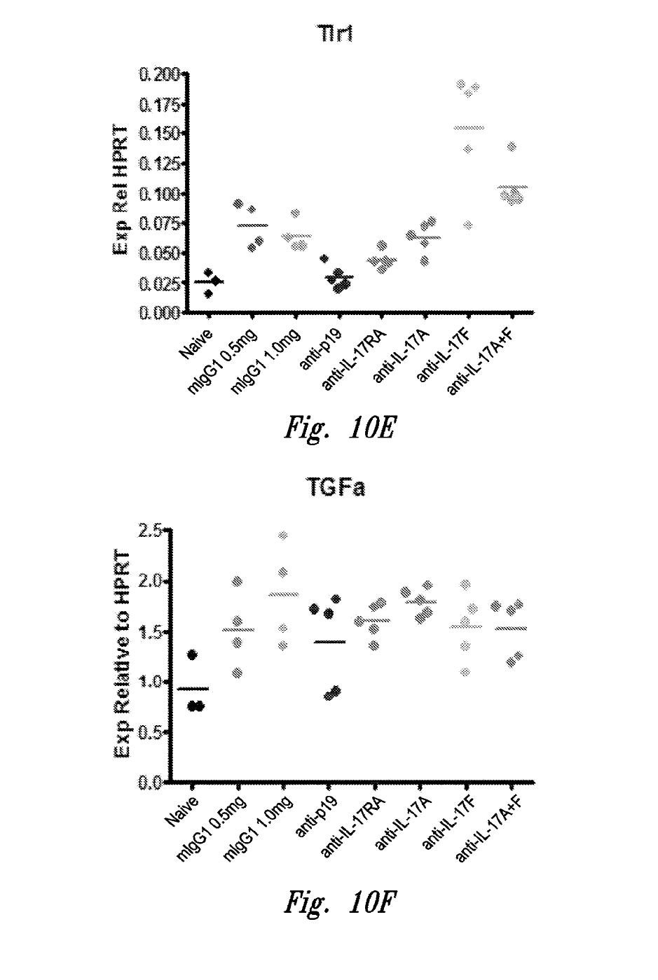

FVB.K14/IL-36α transgenic mice, (8-12 week-old males) were generated and used in a chemical-induced psoriasis model (for details on the TPA mouse model see Blumberg H, et al., J Exp Med. 2007 Oct. 29; 204(11):2603-14 and Blumberg H, et al. J Immunol. 2010 Oct. 1; 185(7):4354-62. Note: IL-1F6 has been renamed to IL-36α). FVB.K14/IL-36αtg mice treated with TPA have been shown to develop skin lesions with histological similarities to human psoriasis and also show an increase in CD31, cytokeratin 6, CD11c and CD3 staining in the skin by immunohistochemistry (Blumberg H, et al. J Immunol. 2010 Oct. 1; 185(7):4354-62). Genes induced in the FVB.K14/IL-36αtg+TPA model overlap many of those observed in human psoriatic lesional skin samples (see Bumberg, 2010, supra and FIGS. 3-10), and such as the genes highlighted as upregulated in human psoriatic skin lesions compared with non-lesional skin (FIG. 31).

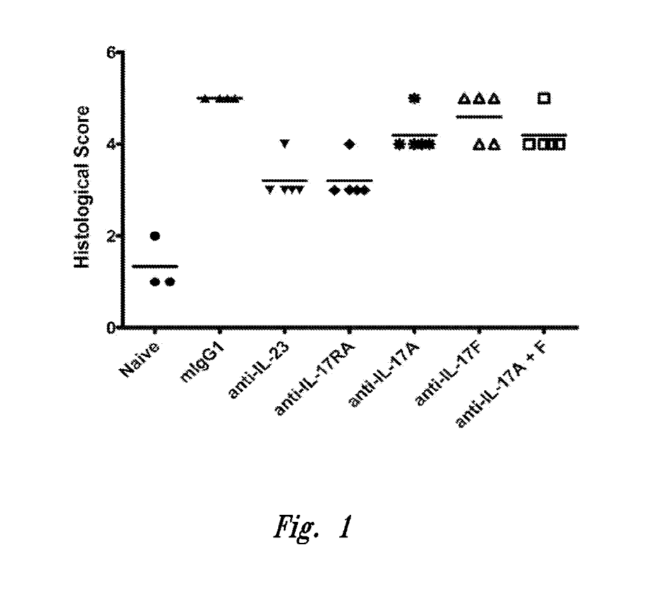

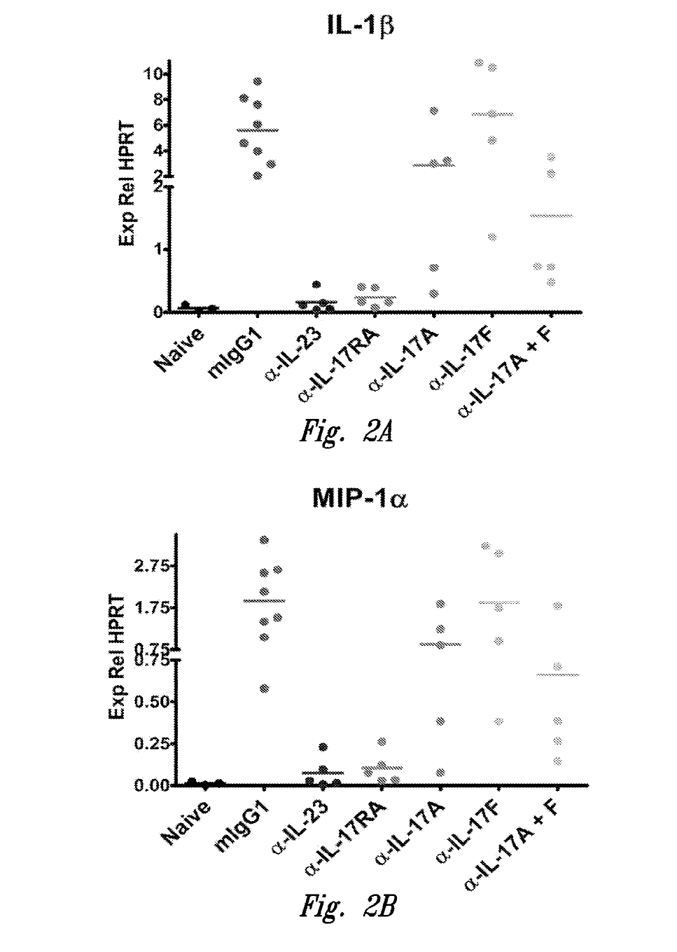

Dorsal hair was shaved 24 hrs prior to TP A (12-O-tetradecanoylphothol-13-acetate) administration. On days 0 and 4, mice (other than the naive control group) received 12.5 ug TPA (Sigma-Aldrich, St. Louis Mo.) in 200 ul acetone by topical administration to the shaved skin. One day prior to TP A administration, on days minus-one and day 3, mice received 500 ug (2.5 mg/ml diluted into PBS, injected 200 ul) of the various antibodies shown in FIGS. 1 and 2 via IP injection. The antibodies to IL-17A, IL-17F, and IL-17RA were known to be neutralizing antibodies. An irrelevant mouse IgG1 mAb was used as a control.

Anti-mouse IL-17RA monoclonal antibody: An IgG mAb to mouse IL-17RA (referred to in the literature as M751) was generated using standard hybridoma techniques in Lewis rats. Briefly, rats were immunized with recombinant mouse IL-17RA.Fc (R&D Systems, Minneapolis, Minn.), spleens and inguinal lymph node cells were fused with NS-1 mouse myeloma cells, and the resulting Ag-positive hybridomas were cloned by limiting dilution. IgG was purified from cell culture supernatants from subcloned hybridoma lines and tested for their ability to block IL-17A-induced IL-6 production from cultured NIH-3T3 cells. A mAb to mouse IL-17RA was chosen for in vivo use that completely blocked IL-17A-induced IL-6 production in the 3T3 cell assay. The chosen antibody was recombinantly cloned by standard techniques and then chimerized by fusing the variable region domain of the rat IgG to mouse IgG1 constant domains. The chimeric antibody was transfected into 293 cells and purified from cell supernatants and tested to confirm activity in the 3T3 assay. This mAb does not bind mouse IL-17RB or mouse IL-17RC by ELISA.

Anti-mouse IL-17A monoclonal antibody: An IgG mAb to mouse IL-17A (referred to in the literature as M210) was generated using standard hybridoma techniques in Lewis rats. Briefly, rats were immunized with recombinant mouse IL-17A.Fc (R&D Systems), spleens and inguinal lymph node cells were fused with NS-1 mouse myeloma cells, and the resulting Ag-positive hybridomas were cloned by limiting dilution. IgG was purified from cell culture supernatants from subcloned hybridoma lines and tested for their ability to block IL-17A-induced IL-6 production from cultured NIH-3T3 cells. A mAb to mouse IL-17A was chosen for in vivo use that completely blocked IL-17A-induced IL-6 production in the 3T3 cell assay. The chosen antibody was recombinantly cloned by standard techniques and then chimerized by fusing the variable region domain of the rat IgG to mouse IgG1 constant domains. The chimeric antibody was transfected into 293 cells and purified from cell supernatants and tested to confirm activity in the 3T3 assay.

Anti-mouse IL-17F monoclonal antibody: An IgG mAb to mouse IL-17RA (referred to in the literature as M850) was generated using standard hybridoma techniques in Lewis rats. Briefly, rats were immunized with recombinant mouse IL-17F.Fc (R&D Systems), spleens and inguinal lymph node cells were fused with NS-1 mouse myeloma cells, and the resulting Ag-positive hybridomas were cloned by limiting dilution. IgG was purified from cell culture supernatants from subcloned hybridoma lines and tested for their ability to block IL-17F-induced IL-6 production from cultured NIH-3T3 cells. A mAb to mouse IL-17F was chosen for in vivo use that completely blocked IL-17F induced IL-6 production in the 3T3 cell assay. The chosen antibody was recombinantly cloned by standard techniques and then chimerized by fusing the variable region domain of the rat IgG to mouse IgG1 constant domains. The chimeric antibody was transfected into 293 cells and purified from cell supernatants and tested to confirm activity in the 3T3 assay.

48 hrs after the second TPA administration (day 6), photos were taken for gross observation, and full-thickness back skin was excised and divided into three sections. The upper and middle portions of the skin samples were snap frozen in liquid nitrogen for RNA and protein analysis. The lower portion of the skin samples were preserved in 10% formalin buffer for histopathology. The skin samples were embedded in paraffin using standard techniques 5-mm sections were cut and mounted onto microscope slides. The slides were stained with using standard hematoxylin and eosin (H&E) methodology. The samples were evaluated through the skin inflammation score system provided below.

Histologic Score (H&E):

0=histologically unremarkable

1=minimal epidermal hyperplasia (focal-multifocal)

2=mild parakeratosis, acanthosis, microabscesses and/or inflammation

3=moderate parakeratosis, acanthosis, microabscesses and/or inflammation

4=marked parakeratosis, acanthosis, microabscesses and/or inflammation

5=severe parakeratosis, acanthosis, microabscesses, inflammation and/or ulceration

As shown in FIG. 1, IL-17RA inhibition was more efficacious than IL-17A inhibition. IL-17A inhibition provided only a partial effect. IL-17RA inhibition was comparable to IL-23 inhibition. Notably, IL-17RA inhibition with an anti-IL-17RA antibody was more effective than inhibiting both IL-17A and IL-17F with respective antibodies. Inhibition of IL-17F had little effect Inhibition of IL-17A or IL-17A plus IL-17F provided an intermediate effect between IL-17F inhibition and IL-17RA inhibition.

### B. Gene Expression Analysis: TPA (12-O-Tetradecanoylphorbol-13-Acetate) Induced IL-36α Transgenic Mouse Skin Inflammation Model

The skin samples that were snap-frozen in liquid nitrogen (see above) were prepared for RNA analysis. A rotor-stator type homogenizer was used to homogenize the skin tissue as follows: 1.5 mis of RLT buffer from the RN Easy® Mini kit (Qiagen, Germantown, Md.) was added to one of the skin tissue samples and was homogenized for approximately 30 seconds to 1 minute, then centrifuged for 10 minutes at 14000 rpm in a micro-centrifuge (4° C.), and the resulting supernatants transferred to new tubes for RNA purification. RNA purification was performed using the reagents and protocol provided in the RNeasy® Mini kit. RNA quantity was measured by NanoDrop™ technology, and quality was analyzed using an Agilent 2100 Bioanalyzer (Agilent Technologies, Palo Alto, Calif.). Only samples yielding intact 18S and 28S ribosomal RNA profiles were used for analysis. 2 ug of total RNA was used to synthesize cDNA from the RNA samples using the reagents and protocols provided in Applied Biosystems' High-Capacity cDNA Reverse Transcription Kits (Applied Biosystems, Foster City, Calif.). cDNA samples were analyzed by TaqMan® low density array (TLDA) using a customized TLDA card with 62 query genes and 2 control genes (i.e., HPRT and 18S). See details below. Custom TLDA cards were provided by Applied Biosystems. TLDA was performed using an Applied Biosystems 7900HT Fast Real Time PCR System, as per manufacturer's instructions. Results were normalized to the expression of the HPRT control gene. Graphs were generated using GraphPad Prism v5™ software.

Custom TLDA card v2 (64 genes):
MCP-1, MIP1-a, MIP1-b, KC(IL-8), IP-10, Foxp3, IL-17C, Rorc, STAT4, IL-17RC, Mmp-13, Mmp-9, Mmp-8, Nos2, Osm, EGF, IL-12rb2, IL-18rap, GMCSF, G-CSF, IL-17D, IL-12p35, IL-17RD, IL-17A, IL-17F, IL-19, IL-20, IL-22, IL-23p19, IL-24, IL-28b, IL-33, IL-17RE, IL-25, IL-6, TGF-a, TNF-α, OX40L, IL-1α, IL-1b, IL-17B, IL-1f5, IL-1f6, IL-1f8, IL-1f9, IL-17RA, IL-17RB, IL-1rn, SIGIRR, Areg, Btc, Defb14, Defb4, Hbegf, Krt6a, S100a8, S100a9, Tlr1, Tlr5, Tnfsf14, Tnfsf15, Zbtb16, HPRT and 18S

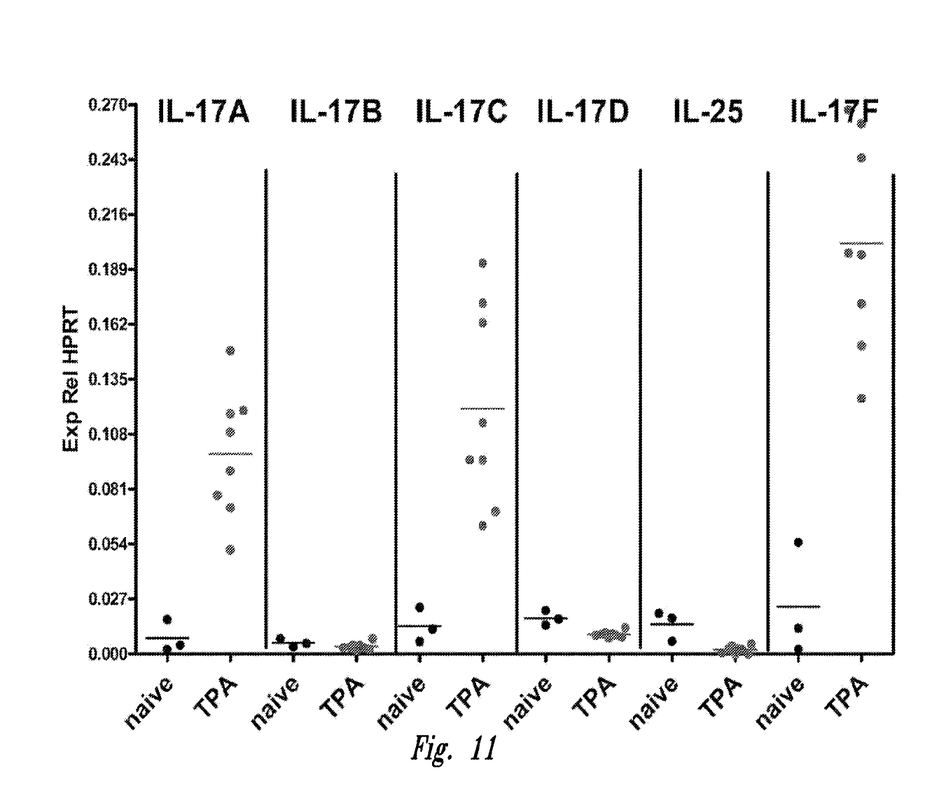

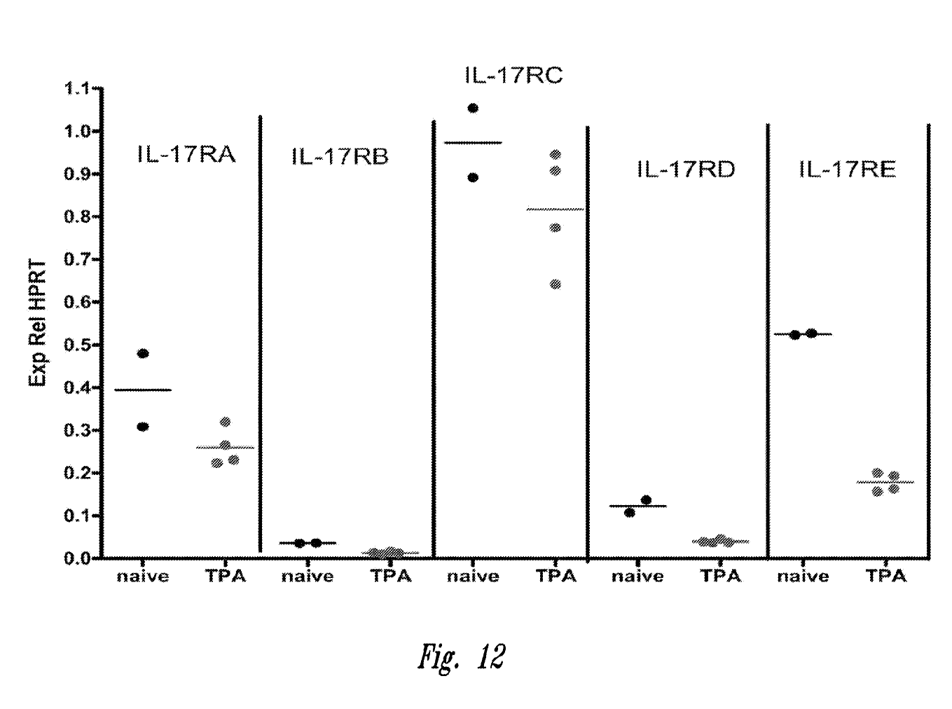

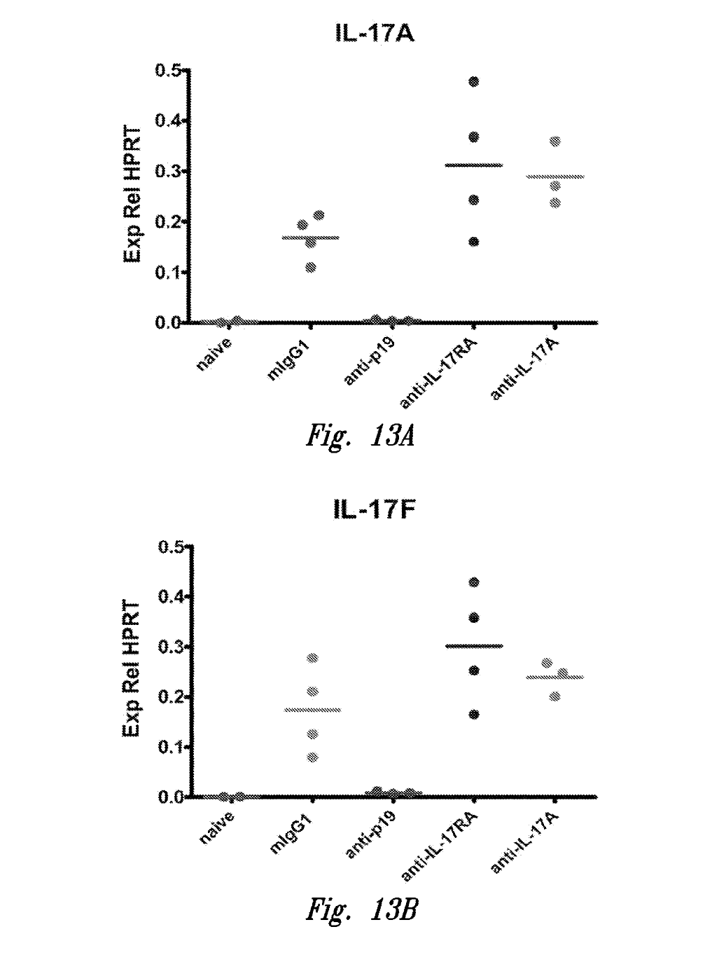

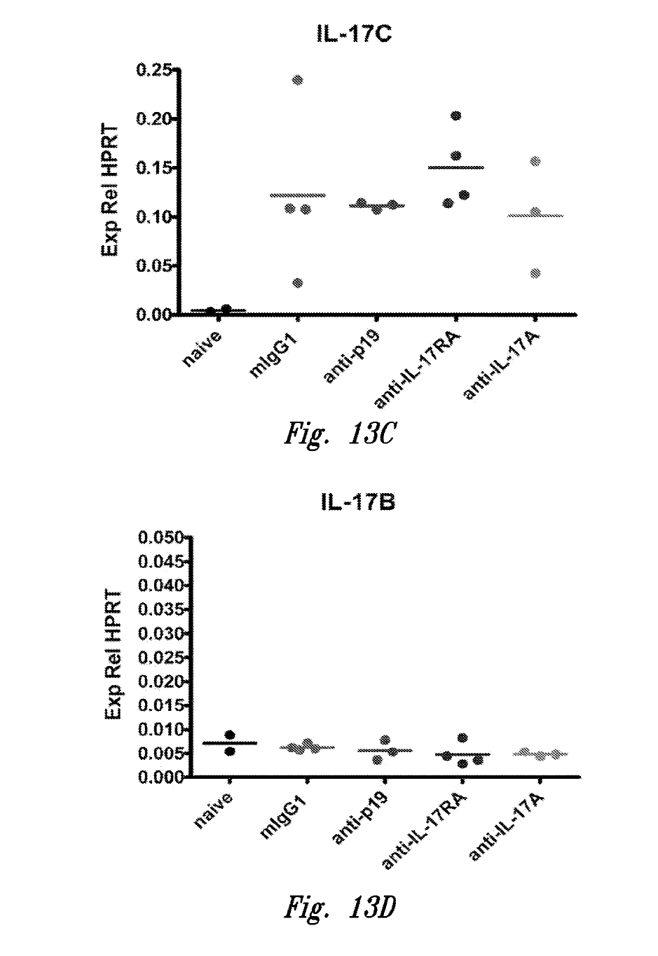

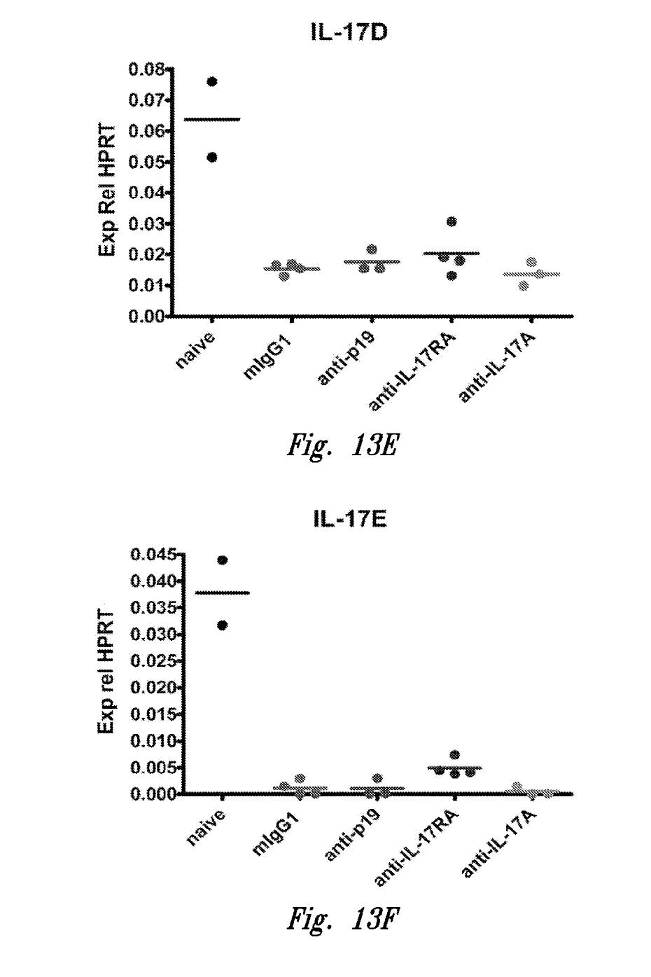

A neutralizing mAb to IL-17RA inhibited expression of many inflammatory genes in the skin, such as IL-1β and MIP-1α (FIG. 2) Similar results were seen with a number of cytokines, chemokines, MMPs and anti-microbial proteins, as shown in FIGS. 3-10. Importantly, the neutralizing mAb to IL-17RA inhibited expression of many genes to a greater extent than neutralizing mAbs to both IL-17A and IL-17F. As shown in FIG. 11, IL-17C was the only other IL-17 family member upregulated in the TP A skin inflammation model. Without being bound by theory, this data suggests that anti-IL-17RA inhibition of IL-17C in the skin inflammation model is the basis for the enhanced efficacy seen with IL-17RA inhibition compared to IL-17A and/or IL-17F inhibition. IL-17RA, IL-17RC, and IL-17RE are expressed in the skin of IL-1F6 Tg mice and the expression of these three receptors appears to decrease by TP A treatment (FIG. 12). Expression of IL-17A, IL-17F as well as IL-17C was increased in the skin of Tg mice treated with TPA, and thereby implicating these cytokines in the inflammation and pathogenesis of this psoriasis model (FIGS. 11 and 13).

### 2. EXPRESSION OF IL-17A, IL-17F AND IL-17C IN THE COLONS OF COLITIC MICE

Mdr1a^{-/-} Colitis Mouse Model: The Mdr1a^{-/-} colitis mouse model is an art-recognized model of IBD/colitis (see Panwala et al. 1998, J Immunol 5733-5744). Mdr1a^{-/-} mice (Taconic Farms, Hudson, N.Y., see Schinkel et al. 1994, Cell 77: 491-502) were gavaged with 1×10⁷ H. bilis bacteria twice, one week apart (provided by Dr. Maggio-Price of the University of Washington; note: strains of H. bilis bacteria are available from ATCC—American Type Culture Collection, Manassas, Va.). The mice were weighed once per week and monitored for clinical symptoms 2-3 times weekly for signs of colitis (e.g., inflammation). At the time of necropsy, sections from the proximal, middle, and distal colon were fixed in 10% neutral buffered formalin and hematoxylin and eosin stained or processed for immunohistochemistry (IHC) staining A portion of the proximal/middle portion of the colon was snap-frozen in liquid nitrogen for RNA isolation.

### A. Colon Tissue Gene Expression Analysis

Colon tissues approximately 0.25-0.5 cm in length were added to 0.8 mls of RLT buffer from Qiagen's RNeasy® mini kit. The tissues were homogenized twice using Qiagen's TissueLyser™ at a setting of 28 hz for 1.5 minutes. The homogenized tissue was centrifuged for 10 minutes at 14000 rpm in a microcentrifuge (4° C.). The resulting supernatants were transferred to new tubes for RNA purification. RNA purification, quantification, and qualification were performed as described above in Example 1. The TLDA analysis was performed using the same custom TLDA card provided by Applied Biosystems as described in Example 1. The data was analyzed and graphed as described in Example 1.

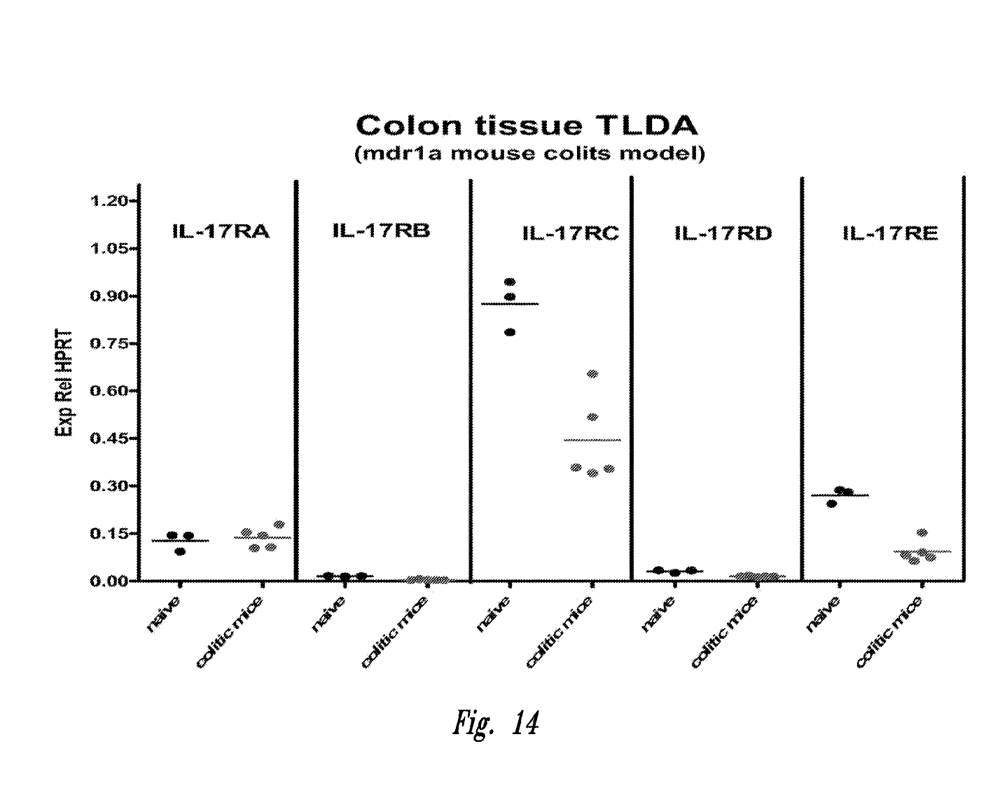

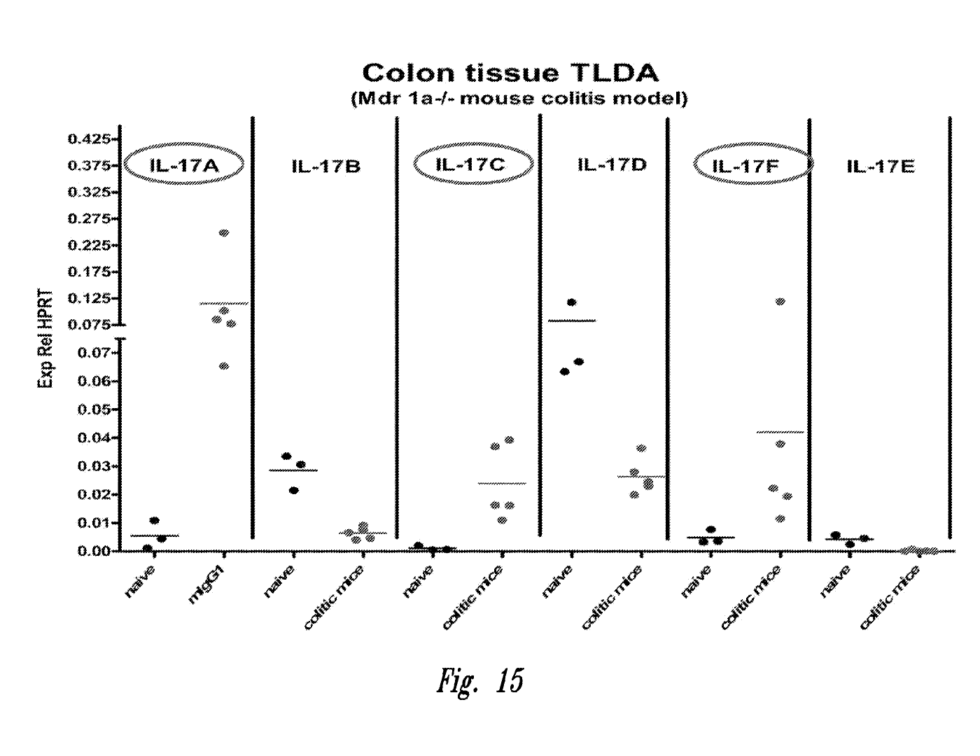

As shown in FIG. 14, IL-17RA, IL-17RE and IL-17RC are expressed in mouse colon tissue. FIG. 15 shows that the expression of IL-17A, IL-17F and IL-17C was increased in the colons of colitic mice compared to that of non-disease mice; that expression of IL-17B, IL-17D and IL-17E was decreased in the colons of colitic mice compared to that of non-disease mice; and that expression of IL-17E was undetectable.

This data shows that IL-17C expression is regulated under conditions of excess inflammation, such as in IBD.

### 3. Expression of IL-17 & IL-17R Family Members in NHEK Cells

NHEK Cell Culture:

Normal human epidermal keratinocytes (Lonza, Basel, CH) were cultured in KGM medium with supplements (KGM-Gold BullefTh₁ kit, Lonza, Basel, CH) at a culture concentration of approximately 1.5×10e4/ml following the manufacture's protocol. At passage 3-4, 1×10e5/ml cells were plated into 6 well tissue culture plates at 2 ml/well. After 24 hrs culturing under standard conditions, the culture medium was removed and replaced with new medium with or without cytokines (duplicate wells for each condition). The cells were cultured for an additional 24 hrs, whereupon the supernatants were harvested for analysis by ELISA and cells were harvested for RNA analysis.

1. Medium only
2. rhuTNF-α (R&D Systems) at 2 ng/ml, 20 ng/ml and 200 ng/ml
3. rhuIL-17A (R&D Systems) at 200 ng/ml
4. rhuIL-17A (R&D Systems) at 200 ng/ml+TNF-α
5. rhuIL-17F (R&D Systems) at 200 ng/ml
6. rhuIL-17F (R&D Systems) at 200 ng/ml+TNF-α
7. rhuIL-17C (R&D Systems) at 200 ng/ml
8. rhuIL-17C (R&D Systems) at 200 ng/ml+TNF-α

RNA purification and TLDA analysis: NHEK cells were lysed by adding 0.6 ml of RLT buffer from the RNeasy® Mini kit (Qiagen) to each well of the 6-well cell culture plate. The plates were placed on ice during processing. The cell lysates were transferred to new tubes for RNA purification. RNA purification was performed using the reagents and protocol provided in the RNeasy® Mini kit (Qiagen, Germantown, Md.). RNA quantity was measured by NanoDrop™ technology and quality was analyzed using an Agilent 2100 Bioanalyzer (Agilent Technologies, Palo Alto, Calif.) according of the manufacturer's protocol. Only samples yielding intact 18S and 28S ribosomal RNA profiles were used for analysis. 2 ug of total RNA was used to synthesize cDNA from the RNA samples using the reagents and protocols provided in Applied Biosystems' High-Capacity cDNA Reverse Transcription Kit (Applied Biosystems, Foster City, Calif.). cDNA samples were analyzed by TaqMan low density array (TLDA) using a customized TLDA plate with 45 query genes and 3 control genes (i.e., HPRT, 18S and GAPDH). See details below. TLDA was performed using an Applied Biosystems 7900HT Fast Real Time PCR System, as per manufacturer's instructions. Results were normalized to the expression of the HPRT control gene. Graphs were generated using GraphPad Prism v5™ software.

Human IL-17 focus TLDA card V 1.1:
CCL2, CCL20, CCL3, CC14, CSF2, CSF3, CXCL1, CXCL10, CXCL14, CXCL2, CXCL3, CXCL5, IL-17A, IL-17B, IL-17C, IL-17D, IL-17F, IL-17E, IL-17RA, IL-17RB, IL-17RC, IL-17RD, IL-17RE, IL-1b, IL-1R2, IL-IRN, iL-20, IL-22, IL-23A, IL-5, IL-6, IL-8, LCN2, MMP13, MMP3, MMP9, S100a8, S100a9, DEFB4, ICAM1, ICAM3, IFNg, TNFa, TNFRSF1a, TNFRSF1b, 18S, HPRT and GRAPH
   Human Inflammation v1 TLDA card:
MCP-1, CD40L, GM-CSF, EBB, FOXp3, GATA3, IFNg, IL-10, IL-12A, IL-12B, IL-13, IL-15, IL-17A, IL-17F, IL-18, IL-18 bp, IL-1a, IL-1b, IL-1R2, IL-RL1, IL-1ra, Il-21, Il-22, IL-23, IL-23R, IL-25, IL-27, IL-32, IL-33, IL-4, IL-5, IL-6, IL-8, IL-9, LTa, MMP13, MMP3, MMP7, MMP8, NOS2, TBX21, TIMP3, TNFα, TNFSF8, TSLP, 18S, GADPH and HPRT

ELISA Analysis of Protein Levels of G-CSF, BD2 and LCN2 from NHEK Cell Culture:

The supernatants from NHEK cell cultures were analyzed for G-CSF, LCN2 and BD2 protein expression through ELISA using the manufacturer's protocols and reagents (human G-CSF ELISA kit, cat# DY214, R&D Systems; human LCN2 ELISA kit, cat# DY1757, R&D Systems; and human BD2 ELISA kit, cat#900-K1172, PeproTech, Rocky Hill, N.J.). The concentrations of G-CSF, LCN2 and BD2 in NHEK cell culture supernatants were calculated from each standard curve respectively.

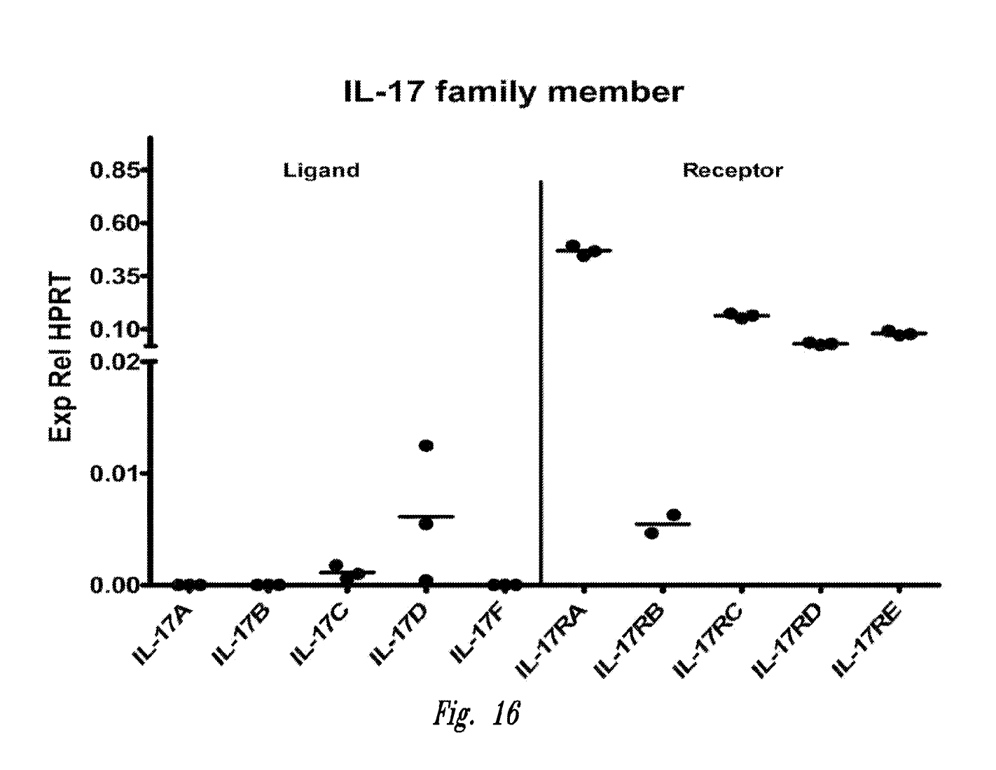

FIG. 16 shows the comparative expression levels of IL-17 and IL-17R family members in NHEK cells (NHEK cells with medium only). IL-17RA, IL-17RC, IL-17RD and IL-17RE are expressed on NHEK cells. IL-17C and IL-17D showed detectable expression.

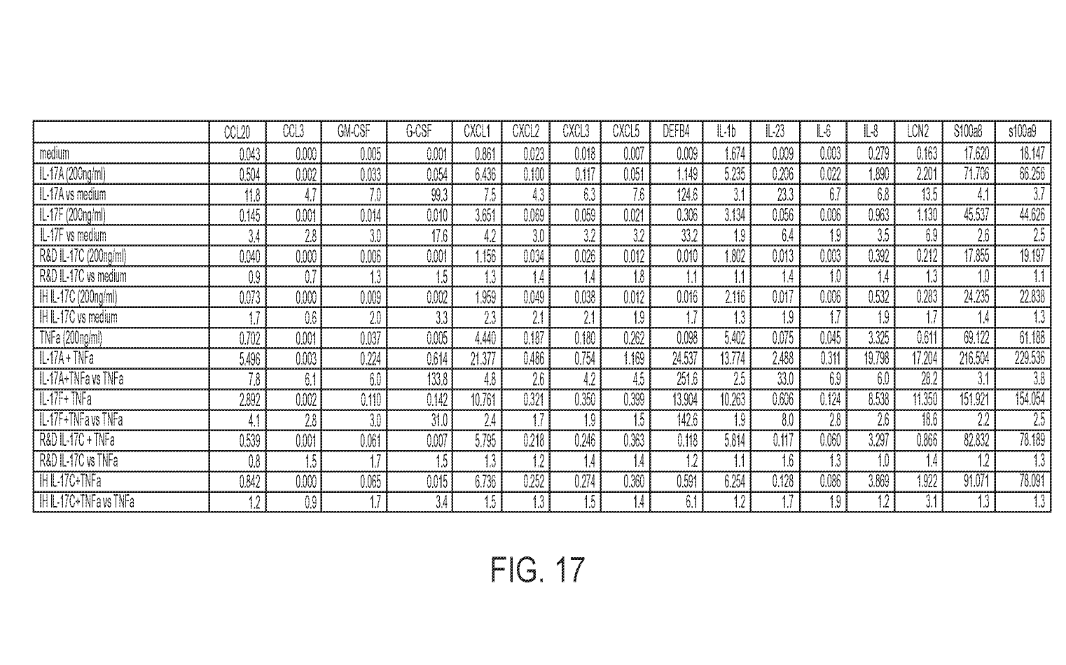

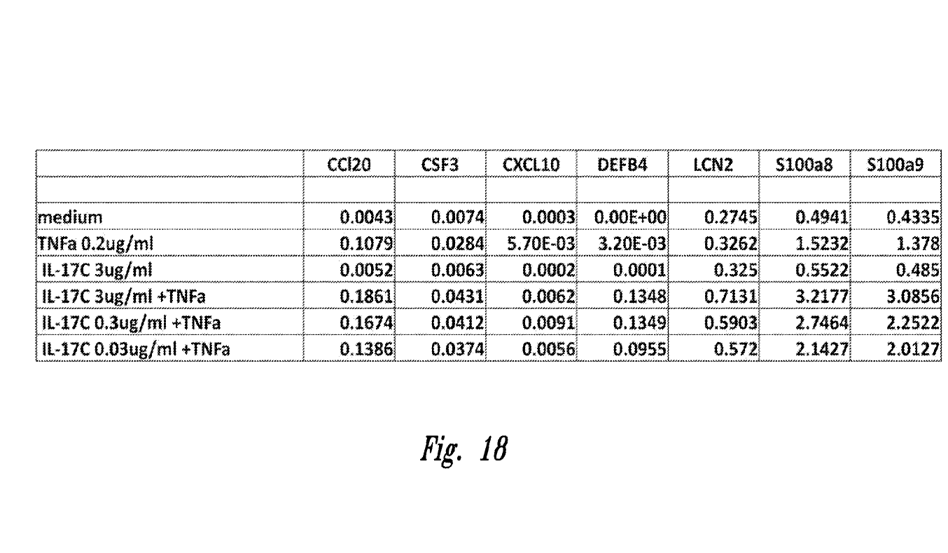

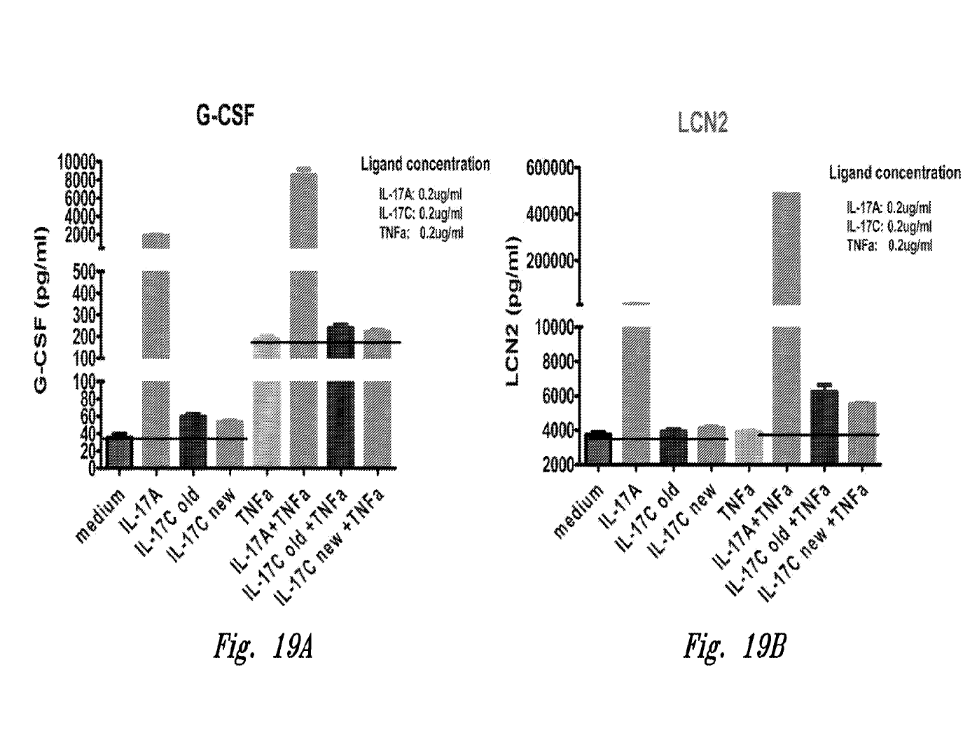

IL-17A and IL-17F induced expression of multiple genes and have strong synergistic effects with TNF-α in the induction of multiple cytokines, chemokines, and anti-microbial peptides from the NHEK cells as compared to IL-17C. The human IL-17 focus v1 was used for TLDA analysis, and the results are presented as the mean of the expression level relative to HPRT for the duplicates of each stimulation condition and as fold-change over medium or TNF-α alone (see FIG. 17). FIGS. 17 and 18 show that IL-17C showed synergistic effects with TNF-α in inducing expression of a limited set of genes such as DEFB4 (data not shown in Figures), lipocalin-2, G-CSF, S100a8 and S100a9 from NHEK cells, and that IL-17C showed small effect in induction of IL-6, IL-8 and GM-CSF from NHEK cells compared to IL-17A and IL-17F. IL-17C treatment resulted in a modest induction of DEFB4, G-CSF and LCN2 protein from NHEK cells and that IL-17C shows an additive effect with TNF-α (FIG. 19).

### 4. Biological Analysis of IL-17C

The prior Examples show that IL-17RA inhibition was highly efficacious in reducing the psoriasis-like skin pathology in the TPA model, while IL-17A-specific inhibition consistently provided a partial effect. The difference in efficacy did not appear to be due to inhibition of IL-17F in that an IL-17F-specific inhibitor had no effect in this model. In addition, adding IL-17A and IL-17F antibodies at the same time was comparable to IL-17A inhibition alone. IL-25 is not expressed in the skin, however it is possible that another IL-17 family cytokine signals through IL-17RA and contributes to the phenotype. IL-17C is the only other IL-17 family cytokine expressed in the skin in this model, and similar to IL-17A and IL-17F, IL-17C is elevated in human psoriatic lesional tissue. IL-17C is reported to bind to IL-17RE, but its biological activity is not well understood.

In order to explore the biological activities of IL-17C we over-expressed IL-17C in mice using a hydrodynamic DNA injection method. Mice over-expressing IL-17C exhibited elevated serum G-CSF concentrations. This IL-17C-induced G-CSF response was lost in mice lacking IL-17RA and was inhibited with an IL-17RA antibody. Antibodies blocking IL-17A, IL-17F, or IL-25 did not significantly affect IL-17C-induced G-CSF. These data suggest that IL-17RA is necessary for IL-17C-induced responses and inhibition of IL-17C in addition to IL-17A could be key to the increased efficacy seen with IL-17RA inhibition compared with IL-17A inhibition in treating psoriasis and potentially other disease states where IL-17C contributes to the pathogenesis.

Mice:

Female BALB/c wild type mice were obtained from Taconic Farms. Female C57BL/6 WT mice were obtained from Charles River Laboratories (Wilmington, Mass.). Female IL-17RA knockout mice were made as previously described (Ye, P., et al, 2001 J. Exp. Med. 194:519-527) and the colony was housed at Taconic Farms, Inc. (Hudson, N.Y.). Experiments were initiated using mice 8-12 weeks old.

Generation of Plasmid DNA Expression Constructs:

Mouse IL-17A (muIL-17A) sequence: NCBI accession number NM_010552.3 was used to make expression construct Igk1::muIL17A::huFC::Flag (note: the C-terminus of the muIL-17A sequence used in these experiments differs from NCBI NM_010552.3 by 2 amino acids. The sequence used in these experiments has a C-terminus ending with amino acids HAS, whereas the NCIBI NM_010552.3 entry dated Jun. 19, 2011 has QAA at the C-terminus). Sense primer 5′AGC TAG CTA ACC GGT GCA GCG ATC ATC CCT CAA 3′ and antisense primer 5′ ATG TGT GAG TTT TGT CGC TTC CAC CGC CTC CGG ACG CAT GGC GGA C3′ was used to generate PCR product (A) containing muIL 17A coding region excluding the leader sequence. Sense primer 5′GTC CGC CAT GCG TCC GGA GGC GGT GGA AGC GAC AAA ACT CAC ACA T3′ and antisense primer 5′CTA GCT AGC TGC GGC CGC CTA TTT ATC ATC ATC ATC TTT ATA ATC TTT ACC CGG AGA CA3′ were used to generate PCR product (B) containing the human FC and Flag tag sequences. Product A and B have complimentary sequences at their respective 3′ and 5′ ends. Final PCR using primers 5′AGC TAG CTA ACC GGT GCA GCG ATC ATC CCT CAA 3′ and 5′CTA GCT AGC TGC GGC CGC CTA TTT ATC ATC ATC ATC TTT ATA ATC TTT ACC CGG AGA CA3′ were used to generate the leaderless coding sequence muIL17A::huFC::Flag; PCR product C. Product C was digested with restriction enzymes Age1 and Not1 and cloned into pENIER1A (Gateway® entry vector, Invitrogen, San Diego, Calif.); cut with Sal1/Not1vector in a 3 way ligation which also included IgK1 sequence digested with Sal1/Age1. The correct clone was identified by DNA sequencing. pENTR1A::IgK1::muI117A::huFC::Flag was Gateway® cloned (Invitrogen, San Diego, Calif.) into pEF100G (destination vector).

Mouse IL-17C (muIL-17C) sequence: NCBI accession number NM_14834.3 was used to make expression construc Igk1::muIL17C::huFC::Flag. Sense primer 5′AGC TAG CAC CGG TGA TCC CCC3′ and antisense primer 5′ATG TGT GAG TTT TGT CGC TTC CAC CGC CTC CCT GTG TAG ACC TGG G3′ was used to generate PCR product (A) containing muIL-17C coding region excluding the leader sequence. Sense primer 5′CCC AGG TCT ACA CAG GGA GGC GGT GGA AGC GAC AAA ACT CAC ACA T3′ and antisense primer 5′CTA GCT AGC TGC GGC CGC CTA TTT ATC ATC ATC ATC TTT ATA ATC TTT ACC CGG AGA CA3′ were used to generate PCR product (B) containing the human FC and Flag tag sequences. Product A and B have complimentary sequences at their respective 3′ and 5′ ends. Final PCR using primers 5′AGC TAG CAC CGG TGA TCC CCC 3′ and 5′CTA GCT AGC TGC GGC CGC CTA TTT ATC ATC ATC ATC TTT ATA ATC TTT ACC CGG AGA CA3′ were used to generate the leaderless coding sequence muIL17C::huFC::Flag; PCR product C. Product C was digested with restriction enzymes Age1 and Not1 and cloned into pENTER1A (Gateway@ entry vector, Invitrogen, San Diego, Calif.); cut with Sal1/Not1vector in a 3 way ligation which also included IgK1 sequence digested with Sal1/Age1. The correct clone was identified by sequencing. pENTR1A::IgK1::muI117C::huFC::Flag was Gateway® cloned (Invitrogen, San Diego, Calif.) into pEF100G (destination vector). pEF100G was used as negative control DNA.

Purification of Plasmid DNA:

Plasmid DNA was recovered by alkaline lysis and subsequently purified by an anion exchange resin per manufacturer's instruction. (Qiagen, Valencia, Calif.). The purity of the plasmid preparations was checked by absorbancy at 260 and 280 nm and 1% agarose gel electrophoresis. Endotoxin was removed from plasmid DNA preparations using Miraclean® Buffer and EndoGO® Extraction kit (Mims Bio, LLC, Madison, Wis.).

Hydrodynamic DNA Injection Experiment:

Plasmid DNA was injected by hydrodynamic technique as previously described (see Liu F, et al., Gene Ther. 1999; 6(7): 1258-1266 and Zhang G, et al., Hum Gene Ther. 1999; 10(10):1735-1737). In brief, 10 μg of endotoxin-free plasmid DNA in sterile Lactated Ringer's Solution (Baxter, Deerfield, Ill.) was delivered in a volume equal to 10% of the mouse body weight by tail vein injection on day 0 (n=3/group/timepoint). Terminal bleeds were performed on days 1, 4, 7, and 11. Administration of the solution was performed in 7 seconds or less without extravasation. The DNA constructs muIL-17A.huFc.FLAG or muIL-17C.huFc.FLAG used an Igkappa leader in place of the native leader to improve expression. The vector pEF100G is Gateway® (invitrogen) adapted and used the human elongation factor 1α promoter. Endotoxin levels were kept below 2UEq/injection.

Blood Collection:

Blood was collected via cardiac puncture during a terminal procedure on days 1, 4, 7 and 11 following DNA injections (mice were injected on day 0). The blood was placed into serum separator tubes, allowed to coagulate, and centrifuged. Serum was stored in clean, labeled tubes at <−20° C. for further analysis.

Western Blot Analysis of muIL-17A.FLAG and muIL-17C.FLAG Proteins:

2.5 μL of serum was brought to 18 μl with the following: 9 μl 2× western sample buffer (Invitrogen LC2676), 1.8 id 10× sample reducing agent (Invitrogen NP0004) and 4.7 μl deionized H₂O. The prepared sera samples were heated 1 min at 95° C. and then separated on 4-20% reducing Tris-glycine gel (40 minutes at 225V), transferred to a PVDF membrane, and blocked overnight at 4° C. in TBST containing 3% nonfat milk After washing in TBST, the membranes were reacted with Clone M2 FLAG antibody conjugated to HRP (Sigma A8592, Sigma-Aldrich Co., St. Louis, Mo.) diluted 1:2000 into TBST for 3 hours at 25° C. After washing with TBST, the membranes were reacted 1 minute with chemiluminescence HRP substrate (ThermoScientific 1859700, Thermo-Fisher Scientific, Inc., Waltham, Mass.). PVDF with serum proteins from muIL-17A expressing mice was exposed to x-ray film for 2 seconds. PVDF with serum proteins from muIL-17C expressing mice was exposed to x-ray film for 5 minutes.

Serum Protein Analytes Analyses:

Serum samples were analyzed by ELISA for G-CSF concentrations using the per the manufacturer's protocols and reagents (R&D Systems, Minneapolis, Minn., human G-CSF ELISA kit, cat# DY214) and by ELISA for huFc concentrations (Syd Labs, Inc., Malden, Mass.). Serum samples were also analyzed using the RodentMAP® v2.0 antigens (Rules-Based Medicine, Austin, Tex.).

Splenocyte assays: Spleens were collected from DNA injected mice and single cell suspensions were prepared. 1 million cells per well were cultured in medium for 72 hours. Splenocyte supernatants were examined using a mouse cytokine 22-plex kit from Millipore (Billerica, Mass.) and analyzed on a Luminex 200 platform (Luminex Corporation, Austin, Tex.) per the manufacturer's recommendations. G-CSF concentrations were confirmed by ELISA (R&D Systems) per the manufacturer's recommendations.

Anti-Mouse IL-17RA Monoclonal Antibody:

An IgG mAb to mouse IL-17RA (referred to in the literature as M751) was generated using standard hybridoma techniques in Lewis rats. Briefly, rats were immunized with recombinant mouse IL-17RA.Fc (R&D Systems, Minneapolis, Minn.), spleens and inguinal lymph node cells were fused with NS-1 mouse myeloma cells, and the resulting Ag-positive hybridomas were cloned by limiting dilution. IgG was purified from cell culture supernatants from subcloned hybridoma lines and tested for their ability to block IL-17A-induced IL-6 production from cultured NIH-3T3 cells. A mAb to mouse IL-17RA was chosen for in vivo use that completely blocked IL-17A-induced IL-6 production in the 3T3 cell assay. The chosen antibody was recombinantly cloned by standard techniques and then chimerized by fusing the variable region domain of the rat IgG to mouse IgG1 constant domains. The chimeric antibody was transfected into 293 cells and purified from cell supernatants and tested to confirm activity in the 3T3 assay. This mAb does not bind mouse IL-17RB or mouse IL-17RC by ELISA.

Anti-Mouse IL-17RB Monoclonal Antibody:

An IgG mAb to mouse IL-17RB (referred to in the literature as M735) was generated in IL-17RB KO mice using standard hybridoma techniques. Briefly, IL-17RB KO mice were immunized with recombinant mouse IL-17RB.huFc (R&D Systems). Spleens and inguinal lymph nodes from mice with positive serum Ab titers to mouse IL-17RB were fused with equal numbers of NS-1 mouse myeloma cells. Ag-positive hybridomas were identified by ELISA and the resulting hybridomas were cloned by limiting dilution. IgG was purified from cell culture supernatants from subcloned hybridoma lines and tested for their ability to block IL-25-induced IL-5 production in a mouse splenocyte assay (Rickel E A, et al., J Immunol. 2008; 181(6):4299-4310). M735 completely blocked IL-25-induced IL-5 production in a mouse splenocyte assay.

Anti-Mouse IL-17A Monoclonal Antibody:

An IgG mAb to mouse IL-17A (referred to in the literature as M210) was generated using standard hybridoma techniques in Lewis rats. Briefly, rats were immunized with recombinant mouse IL-17A.F c (R&D Systems), spleens and inguinal lymph node cells were fused with NS-1 mouse myeloma cells, and the resulting Ag-positive hybridomas were cloned by limiting dilution. IgG was purified from cell culture supernatants from subcloned hybridoma lines and tested for their ability to block IL-17A-induced IL-6 production from cultured NIH-3T3 cells (Chu, C. Q., et al., 2007 Arthritis Rheum. 56: 1145-1151). M210 completely blocked IL-17A-induced IL-6 production in the 3T3 cell assay. The chosen antibody was recombinantly cloned by standard techniques and then chimerized by fusing the variable region domain of the rat IgG to mouse IgG1 constant domains. The chimeric antibody was transfected into 293 cells and purified from cell supernatants and tested to confirm activity in the 3T3 assay.

Anti-Mouse IL-25 Monoclonal Antibody:

An IgG mAb to mouse IL-25 (referred to in the literature as M819) was generated using standard hybridoma techniques in Lewis rats. Briefly, Lewis rats were immunized with mouse IL-25 (R&D Systems). Spleen and inguinal lymph node cells from rats with a positive serum Ab titer were fused with NS-1 mouse myeloma cells, and the resulting Ag-positive hybridomas were cloned by limiting dilution. IgG was purified from cell culture supernatants from subcloned hybridoma lines and tested for their ability to block IL-25-induced IL-5 production in a mouse splenocyte assay (Rickel E A, et al., J Immunol. 2008; 181(6):4299-4310). M819 completely blocked IL-25-induced IL-5 production in a mouse splenocyte assay. M819 was recombinantly cloned by standard techniques and then chimerized by fusing the variable region domain of the rat IgG to mouse IgG1 constant domains. The chimeric antibody was transfected into 293 cells and purified from cell supernatants and tested to confirm activity in the mouse splenocyte assay.

Anti-Mouse IL-17F Monoclonal Antibody:

An IgG mAb to mouse IL-17F (referred to in the literature as M850) was generated using standard hybridoma techniques in Lewis rats. Briefly, rats were immunized with recombinant mouse IL-17F.Fc (R&D Systems), spleens and inguinal lymph node cells were fused with NS-1 mouse myeloma cells, and the resulting Ag-positive hybridomas were cloned by limiting dilution. IgG was purified from cell culture supernatants from subcloned hybridoma lines and tested for their ability to block IL-17F-induced IL-6 production from cultured NIH-3T3 cells (Chu, C. Q., et al., 2007 Arthritis Rheum. 56: 1145-1151). M850 completely blocked IL-17F-induced IL-6 production in the 3T3 cell assay. M850 was recombinantly cloned by standard techniques and then chimerized by fusing the variable region domain of the rat IgG to mouse IgG1 constant domains. The chimeric antibody was transfected into 293 cells and purified from cell supernatants and tested to confirm activity in the 3T3 assay.

### Results

### Expression of Mouse IL-17C in Mice Results in Increased Serum G-CSF Concentrations

To investigate the activity of IL-17C in vivo, we developed plasmid DNA constructs that induced a transient expression of either mouse IL-17A or mouse IL-17C when injected into mice using a hydrodynamic DNA injection method (Liu et al, 1999, supra; Zhang et al, 1999, supra). Both mouse IL-17C and mouse IL-17A DNA constructs included FLAG and huFc tags. Western blot analysis using an anti-FLAG antibody revealed peak serum expression of both IL-17A and IL-17C proteins in DNA injected mice 4 days post injection and protein expression was detectable up to 10 days after injection.

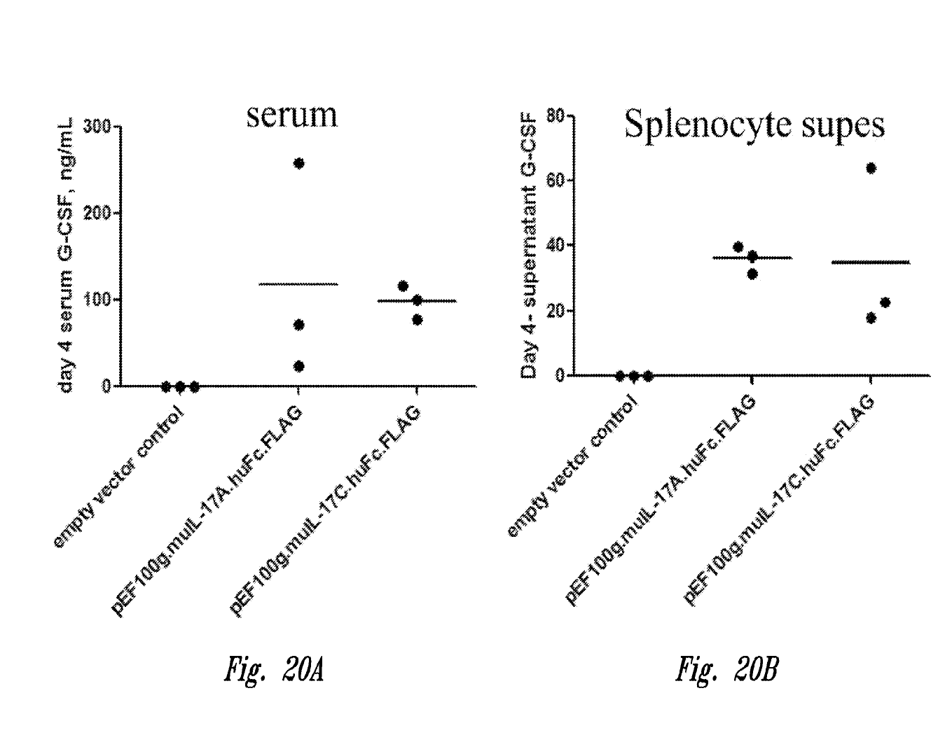

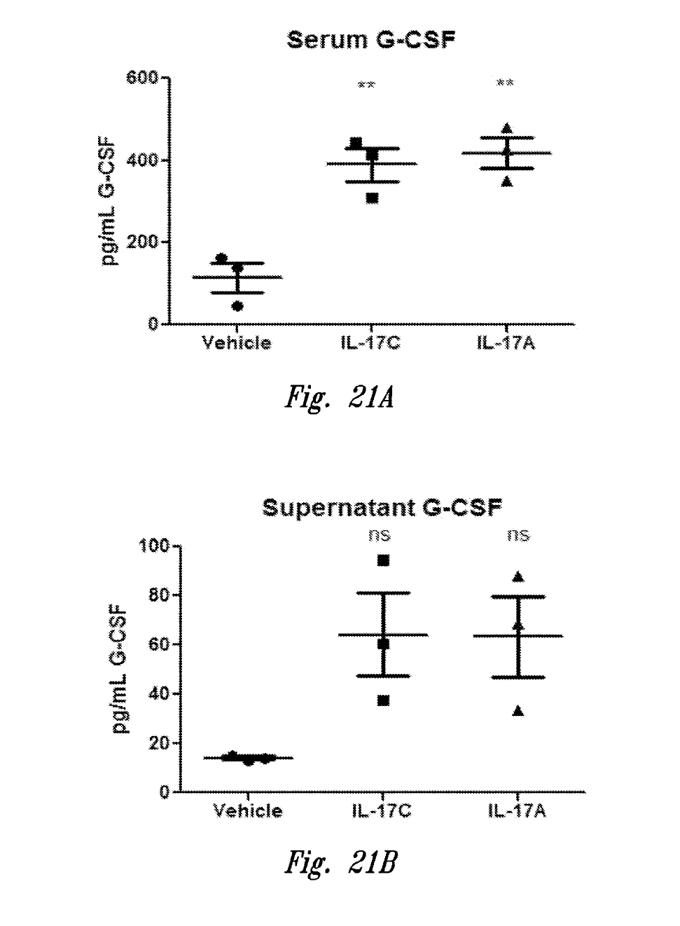

Serum concentrations of various analytes were measured using a Luminex 22-plex assay (described above) in mice that were injected with IL-17A or IL-17C DNA constructs 4 days after injection. Of the analytes measured in the Luminex 22-plex assay, G-CSF serum concentrations were the only analyte significantly increased in mice expressing either mouse IL-17C or mouse IL-17A compared with mice who received a negative control injection of empty DNA vector (pEF 1 OOG) (FIG. 20). The increased serum G-CSF serum concentrations in mice expressing mouse IL-17A or mouse IL-17C were confirmed by ELISA (FIG. 21). Expression of mouse IL-17C did not cause increased IL-17A serum concentrations in mice, providing evidence that IL-17C-induced GCSF is independent of IL-17A.

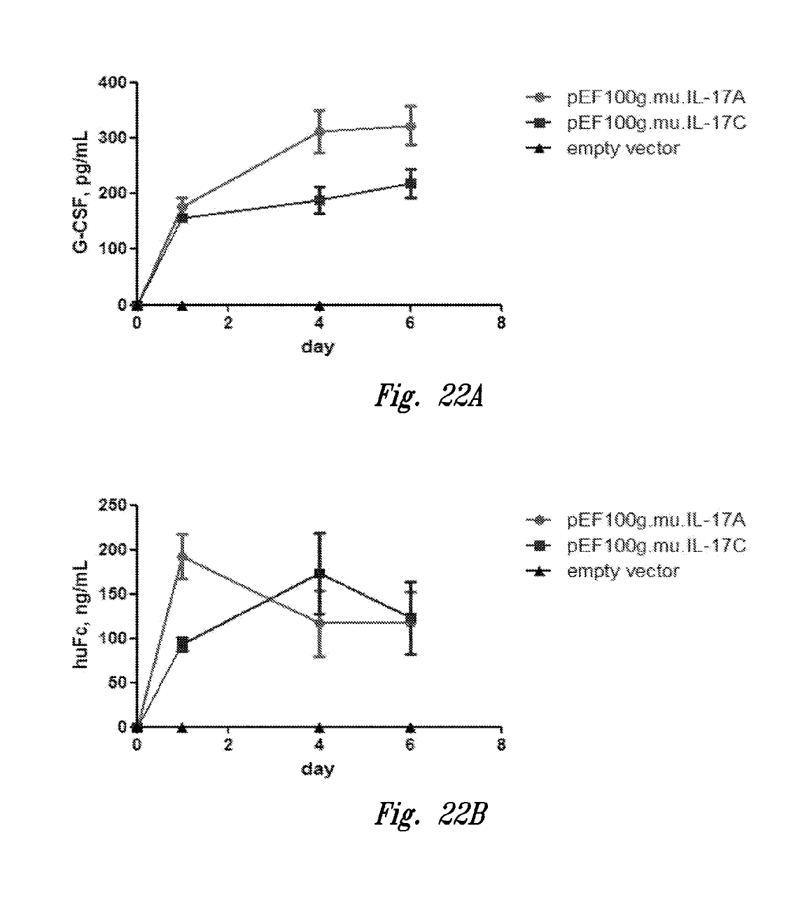

Serum G-CSF and huFc concentrations were both monitored in mice 1, 4, and 6 days following DNA injection. Peak huFc expression (protein tag on IL-17C and IL-17A) was seen on day 1 for IL-17A and on day 4 for IL-17C and persisted through day 6 (FIG. 22). G-CSF serum concentrations peaked for IL-17C on day 1 and persisted through day 6, while for IL-17A, peak GCSF serum concentrations were noted on day 4 and persisted through day 6 (FIG. 22). These data show that IL-17C and IL-17A protein expression is high within 1 day of DNA injection and G-CSF concentrations increase within the same time period of IL-17C or IL-17A protein expression.

Splenocytes Cultured In Vitro from Mice Expressing Mouse IL-17C Produced Increased Levels of G-CSF

Spleens from DNA injected mice were collected 4 days after DNA injection and single cell suspensions from the spleens were cultured in medium for 72 hours. G-CSF concentrations in the splenocyte cell culture supernatants from mice expressing mouse IL-17C or mouse IL-17A were significantly increased compared with splenocyte supernatants from mice who received a negative control DNA injection (FIG. 20).

### IL-17RA is Required for IL-17C-Induced G-CSF

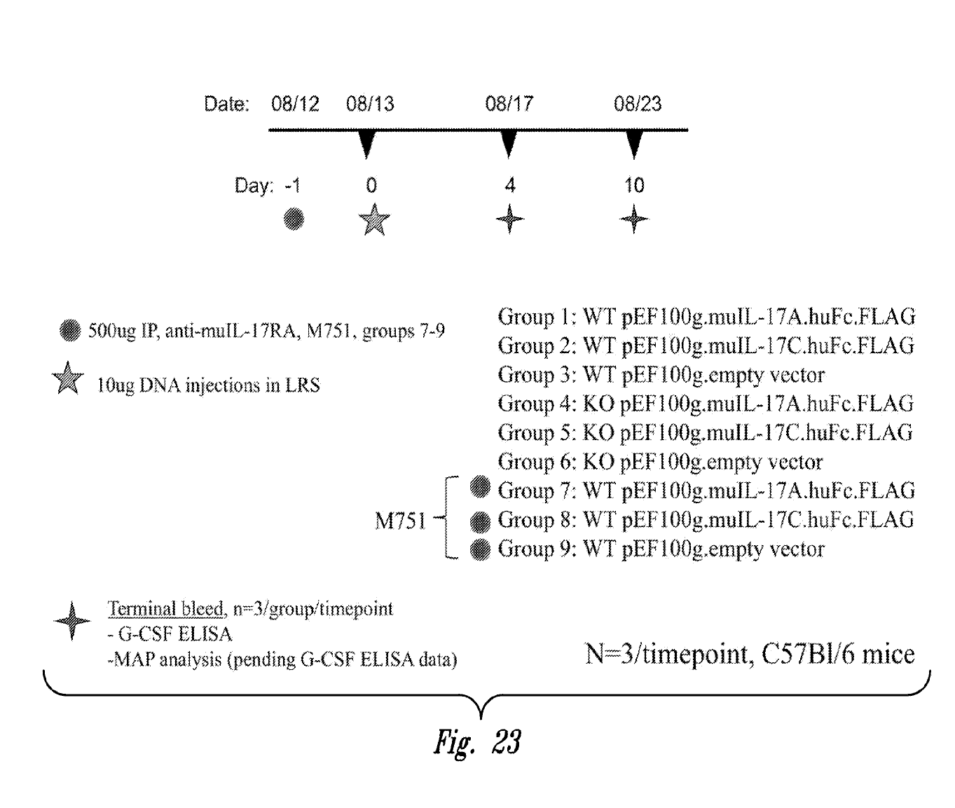

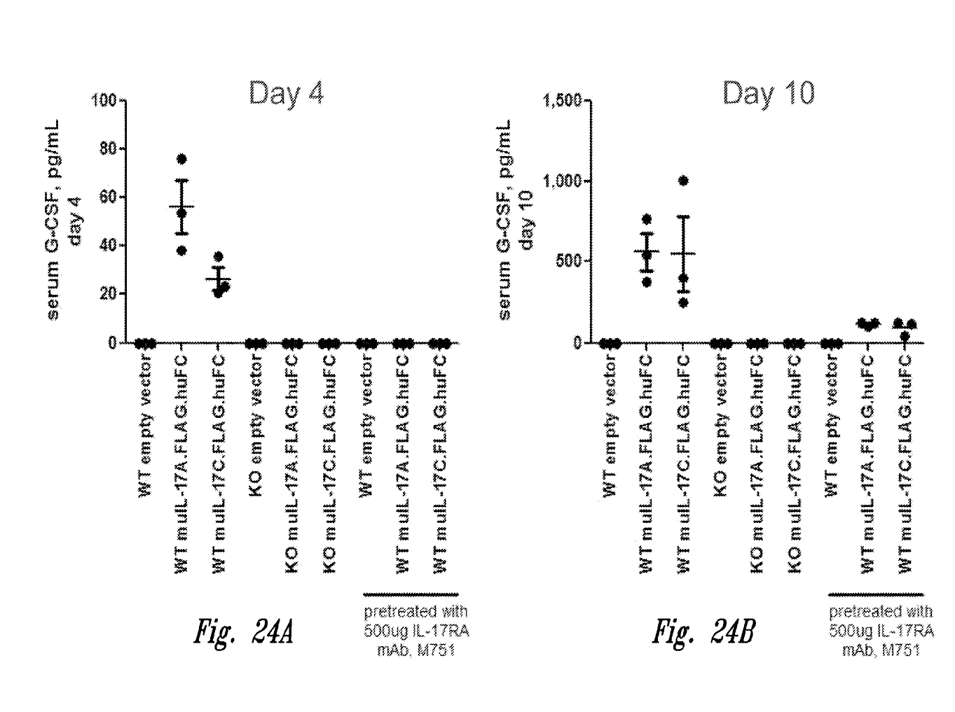

It has been demonstrated that IL-17A signals through a heteromeric receptor complex consisting of IL-17RA and IL-17RC while IL-25 signals through a heteromeric receptor consisting of IL-17RA and IL-17RB (Toy et al, 2006, supra; Rickel et al, 2008, supra). In order to determine if IL-17C also requires IL-17RA, we measured serum G-CSF concentrations from wild type C57B1/6 mice and IL-17RA knockout (KO) mice (also on a C57B1/6 background) that had been injected with either mouse IL-17C or mouse IL-17A DNA constructs (FIG. 23). To further test the role of IL-17RA in IL-17C-induced G-CSF in mice, groups of wild type mice injected with mouse IL-17C or mouse IL-17A DNA constructs were treated with a neutralizing antibody to mouse IL-17RA (M751) the day before DNA injection (FIG. 23) Similar to what was seen in previous experiments described above (FIGS. 20, 21, and 22), wild type C57B1/6 mice expressing mouse IL-17C or mouse IL-17A had significantly higher serum G-CSF concentrations compared with wild type mice receiving a negative control DNA injection (FIG. 24). IL-17RA KO mice expressing mouse IL-17C or mouse IL-17A did not produce any serum G-CSF concentrations that were detectable by ELISA, similar to that seen in mice injected with negative control DNA (FIG. 24). Furthermore, wild type mice treated with anti-IL-17RA mAb M751expressing mouse IL-17C or mouse IL-17A did not produce any serum GCSF concentrations that were detectable by ELISA (FIG. 24). These results provide the first data showing that IL-17RA is required for IL-17C-induced biological activities.

Serum samples from wild type C57B1/6 mice and IL-17RA knockout (KO) mice that had been injected with either mouse IL-17C or mouse IL-17A DNA constructs were analyzed 4 days and 10 days after DNA injection by multiplex analyte profiling for concentrations of 58 different proteins, as shown below.

### RodentMAP® v2.0 Antigens:

Apolipoprotein A-I, C-Reactive Protein, CD40, CD40 Ligand, Endothelin-I, Eotaxin, Epidermal Growth Factor Mouse, Factor VII, Fibrinogen, Fibroblast Growth Factor 9, Fibroblast Growth Factor basic, Glutathione S-Transferase alpha, Granulocyte Chemotactic Protein-2, Granulocyte-Macrophage Colony-Stimulating Factor, Growth-Regulated Alpha Protein, Haptoglobin, Immunoglobulin A, Interferon gamma, Interferon gamma Induced Protein 10, Interleukin-1 alpha, Interleukin-I beta, Interleukin-2, Interleukin-3, Interleukin-4, Interleukin-5, Interleukin-6, Interleukin-7, Interleukin-IO, Interleukin-11, Interleukin-12 Subunit p70, Interleukin-17A, Leukemia Inhibitory Factor, Lymphotactin, Macrophage Colony-Stimulating Factor-1, Macrophage-Derived Chemokine, Macrophage Inflammatory Protein-1 alpha, Macrophage Inflammatory Protein-1 beta, Macrophage Inflammatory Protein-1 gamma, Macrophage Inflammatory Protein-2, Macrophage Inflammatory Protein-3 beta, Matrix Metalloproteinase-9, Monocyte Chemotactic Protein 1, Monocyte Chemotactic Protein 3, Monocyte Chemotactic Protein-5, Myeloperoxidase, Myoglobin, Oncostatin-M, RANTES, Serum Amyloid P-Component, Serum Glutamic Oxaloacetic Transaminase, Stem Cell Factor, Thrombopoietin, Tissue Factor, Tissue Inhibitor of Metalloproteinases 1, Tumor Necrosis Factor alpha, Vascular Cell Adhesion Molecule-1, Vascular Endothelial Growth Factor A, von Willebrand factor

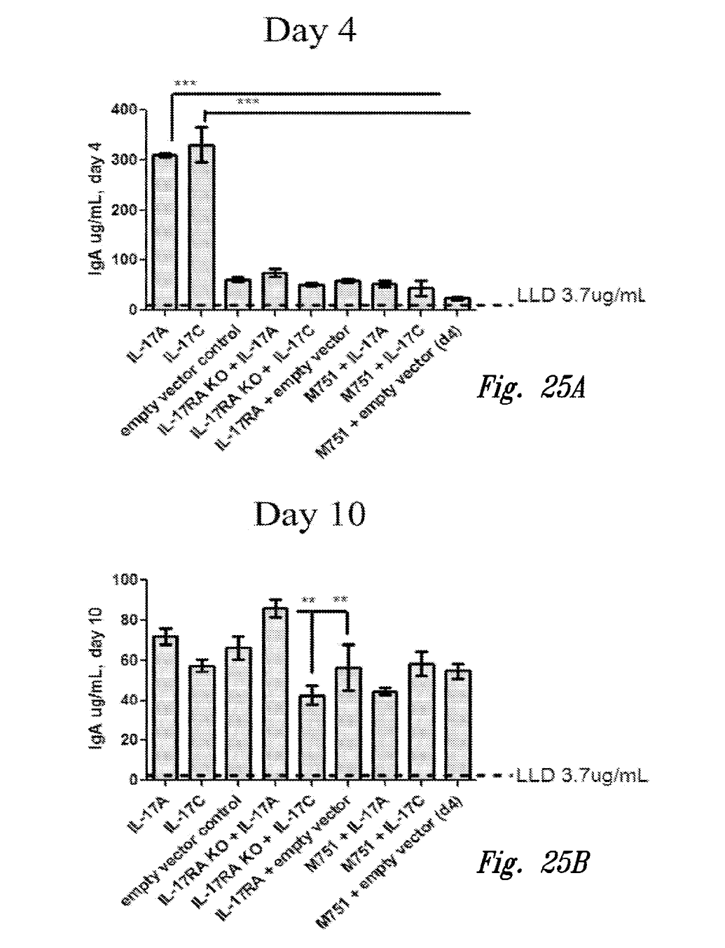

On day 4, wild type mice expressing mouse IL-17C or mouse IL-17A DNA produced significantly more IgA compared with wild type mice receiving a negative control DNA injection, and IgA concentrations were not elevated by either IL-17C or IL-17A expression in IL-17RA KO mice (FIG. 25). Day 4 serum samples from wild type mice treated with anti-IL-17RA mAb M751 prior to IL-17C or IL-17A DNA injection also had significantly less IgA (FIG. 25). Serum IgA concentrations were not significantly higher 10 days after DNA injection in IL-17C or IL-17A expressing mice compared with mice receiving a negative control DNA injection (FIG. 25). These results suggest that IL-17RA is required for IL-17C and IL-17A-induced IgA 4 days after DNA injection.

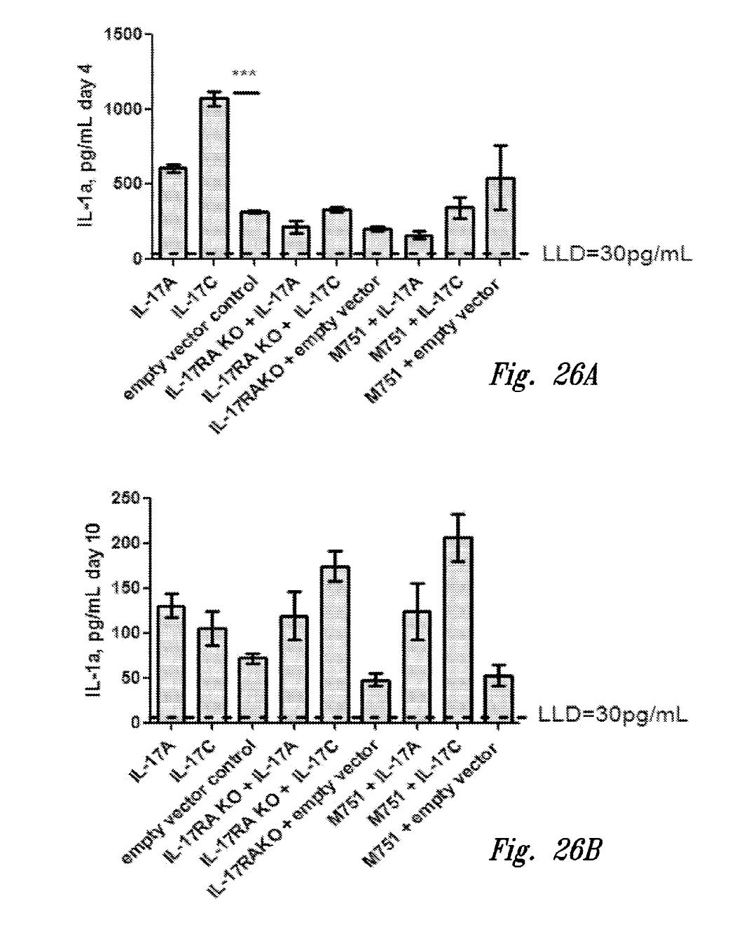

On day 4, wild type mice expressing mouse IL-17C or mouse IL-17A DNA produced significantly more IL-1α compared with wild type mice receiving a negative control DNA injection, and IL-1α concentrations were not elevated by either IL-17C or IL-17A expression in IL-17RA KO mice (FIG. 26). Day 4 serum samples from wild type mice treated with anti-IL-17RA mAb M75I prior to IL-17C or IL-17A DNA injection also had significantly less IL-1α (FIG. 26). Serum IL-1α concentrations were not significantly higher 10 days after DNA injection in IL-17C or IL-17A expressing mice compared with mice receiving a negative control DNA injection. These results suggest that IL-17RA is required for IL-17C and IL17A-induced IL-1α 4 days after DNA injection.

### IL-17a, IL-25, IL-17RB and IL-17F are not Required for IL-17C-Induced G-CSF

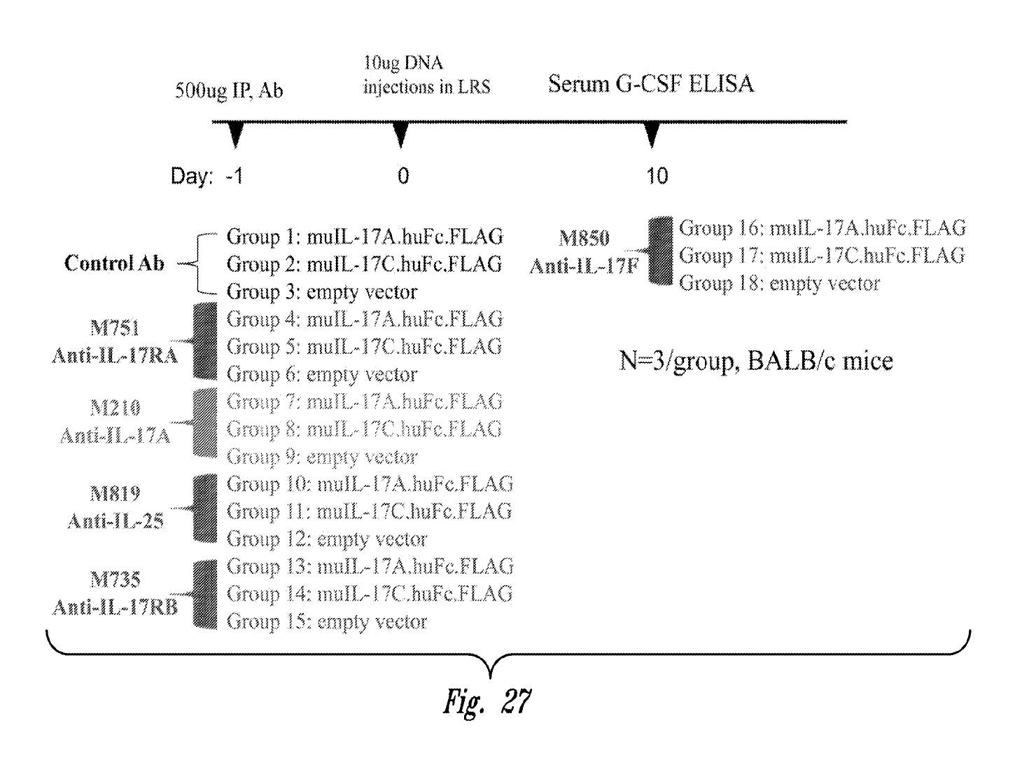

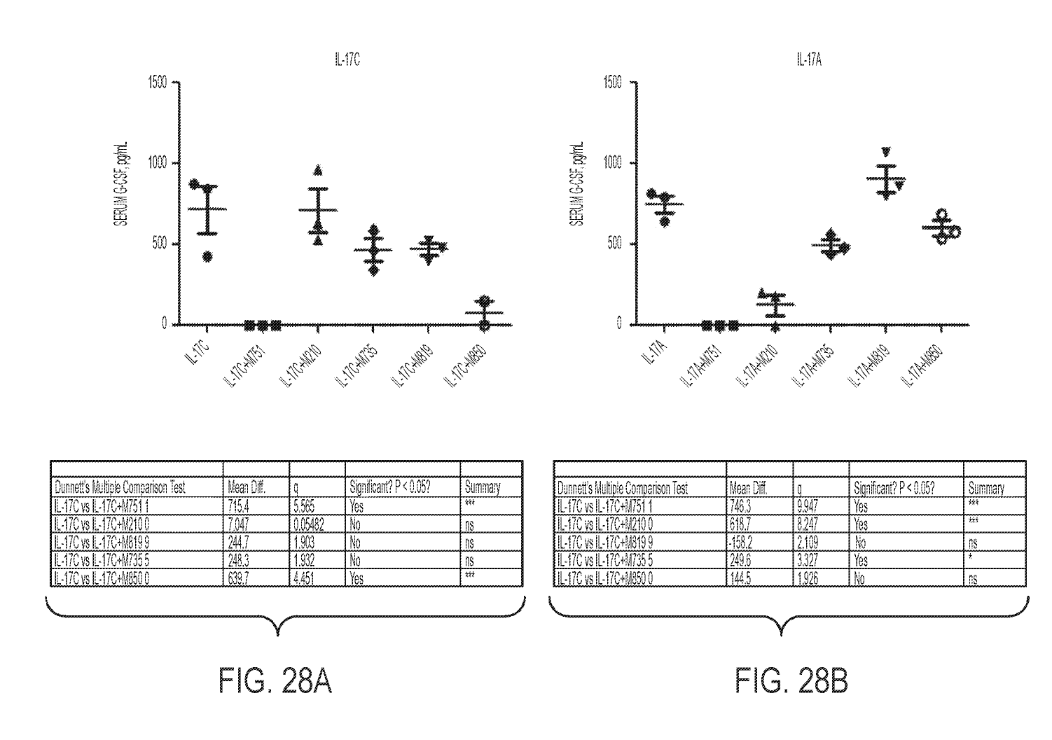

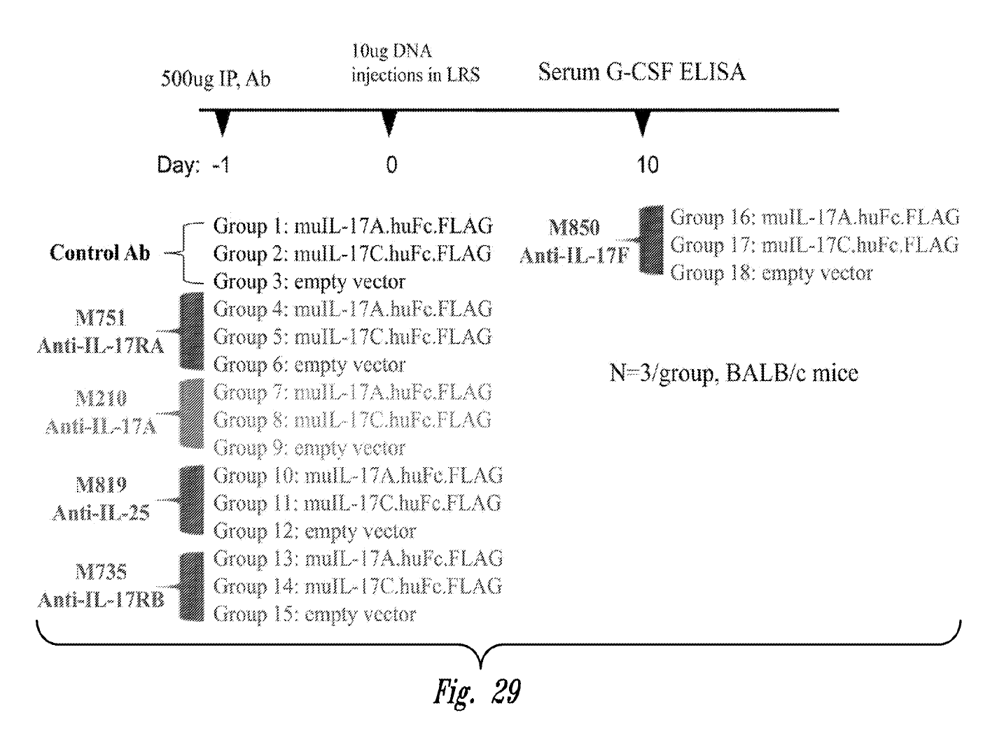

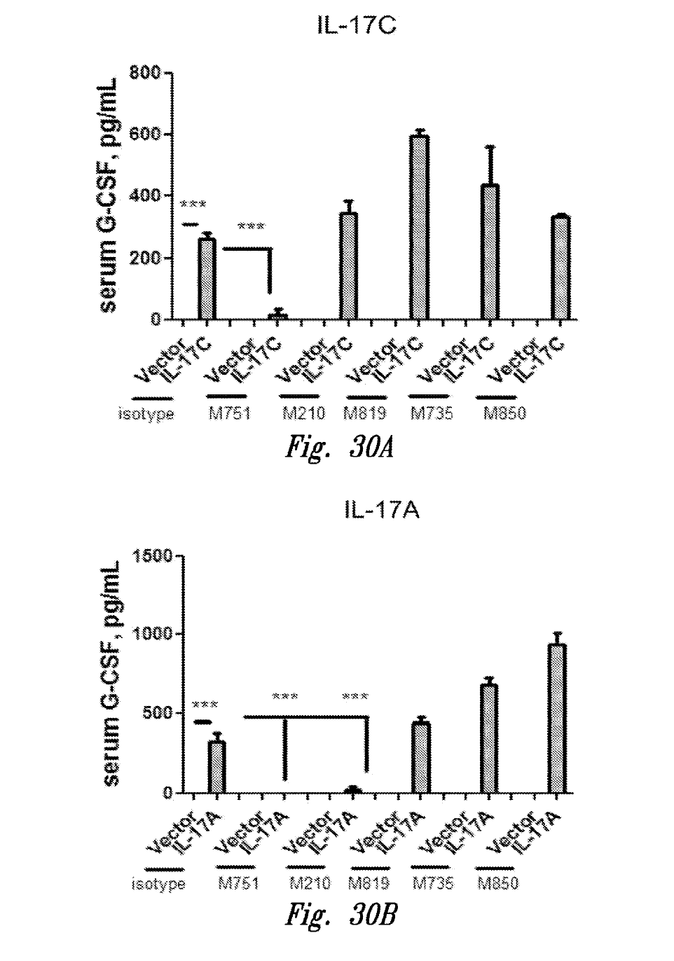

We next explored whether IL-17C-induced G-CSF was dependent on other IL-17 cytokine or receptor family members. Serum G-CSF concentrations were measured in mice 10 days after injection with mouse IL-17C DNA or IL-17A DNA (FIG. 27). The day before DNA injection, mice were injected with either neutralizing antibodies to mouse IL-17RA (M751), IL-17A (M210), IL-25 (M819), IL-17F (M850), or IL-17RB (M735) or a control mouse IgG1 antibody (FIG. 27). Treatment with anti-IL-17RA (M751) significantly reduced IL-17C and IL-17A-induced G-CSF serum concentrations (FIG. 28), similar to what was described above (FIG. 24). Treatment with anti-IL-17A (M210), anti-IL-25 (M819), or anti-IL-17RB (M735) had no significant effect in reducing IL-17C-induced serum G-CSF concentrations (FIG. 28). Treatment with anti-IL-17A (M210) significantly reduced IL-17A-induced G-CSF serum concentrations (FIG. 28). Treatment with anti-IL-25 (M819) or anti-IL-17RB (M735) had no significant effect in reducing IL-17A-induced serum G-CSF concentrations (FIG. 28). The data from mice treated with anti-IL-17F (M850) were not conclusive because of sampling problems (FIG. 28). The experiment was repeated and showed that anti-IL-17RA (M751) significantly reduced IL-17C and IL-17A-induced G-CSF serum concentrations while anti-IL-17A (M210), anti-IL-25 (M819), anti-IL-17F (M850), or anti-IL-17RB (M735) had no significant effect in reducing IL-17C-induced G-CSF serum concentrations (FIGS. 29 and 30). Treatment with anti-IL-17A (M210) significantly reduced IL-17A-induced G-CSF serum concentrations (FIG. 30). Treatment with anti-IL-25 (M819), anti-IL-17F (M850) or anti-IL-17RB (M735) had no significant effect in reducing IL-17A-induced serum G-CSF concentrations (FIG. 28). These data provide the first evidence that IL-17C-induced serum G-CSF concentrations are dependent on IL-17RA and are not dependent on IL-17A, IL-25, IL-17F or IL-17RB.

### 5. IL-17C binds IL-17RE

IL-17 family ligands and receptors were tested in pair wise combinations by Alphalisa™ in Perkin-Elmer's immunoassay buffer. Ligands were tagged with a hexa-histidine tag and receptors were tagged with a FC region.

Reagents: OptiPlate-384™, White Opaque 384-well MicroPlate; Perkin Elmer; Catalog #6007299; AlphaScreen™ Protein A Acceptor beads, 5 mg; Perkin Elmer; Catalog #6760137M; AlphaScreen® Nickel Chelate Donor beads 5 mg; Perkin Elmer; Catalog# AS101M; IL-17C-His-Avi (Amgen); IL-17A-His (Amgen); IL-17RE-FC (Amgen); and IL-17RA-FC (R&D Systems) Catalog#177-IR.

AlphaScreen™ protocol: Ligand and receptor dilutions were made in Perkin-Elmer ImmunoAssay Buffer. IL-17 ligands were diluted to a concentration range of 0.5 to 50 nM. IL-17 receptors were diluted to a concentration range of 0 to 300 nM. Donor and acceptor beads were held constant at 20 ug/mL. 2.5 ul of ligand was added to the 384 well OptiPlate™. 2.5 ul of receptor was added to the 384 well OptiPlate™. 5 uL of donor and acceptor beads were added to the 384 well OptiPlate™. The plate was sealed with adhesive according to manufacturer's protocol. The plate was incubated at room temperature for 3 hours and protected from ambient light. The plate was read using the 384 AlphaScreen Protocol on Envision Plate Reader (Perkin Elmer).

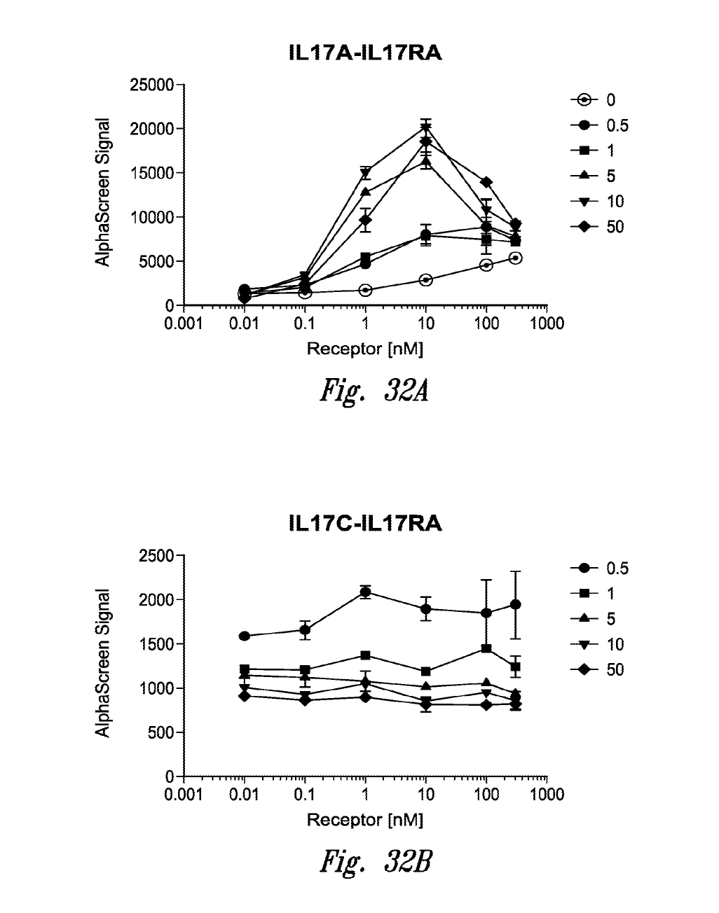

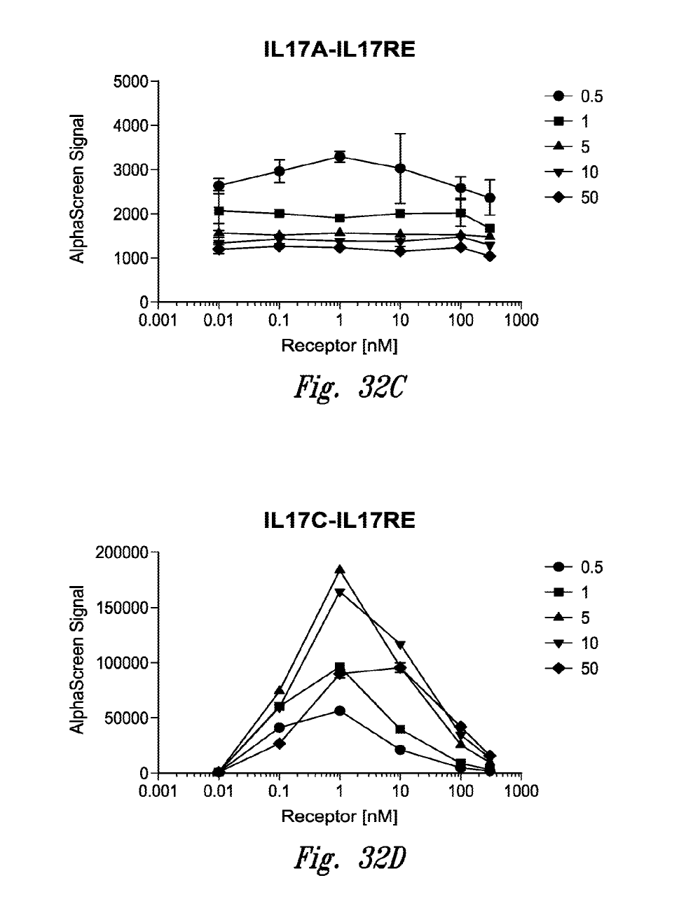

FIG. 32 shows that IL-17A bound to IL-17RA but not to IL-17RE, and that IL-17C bound to IL-17RE but not to IL-17RA.

### 6. Inhibition of IL-17C Activity

NHEK cell culture: Normal human epidermal keratinocytes (Lonza, Basel, CH) were cultured m KGM medium with supplements (KGM-Gold Bullet™ kit, Lonza, Basel, CH) at a culture concentration of approximately 1.5×10e4/ml following the manufacture's protocol. At passage 3-4, 1×10e5/ml cells were plated into 6 well tissue culture plates at 2 ml/well. After 24 hrs culturing under standard conditions, the culture medium was removed and replaced with new medium with or without cytokines (duplicate wells for each condition). The cells were cultured for an additional 24 hrs, whereupon the supernatants were harvested for analysis by ELISA and cells were harvested for RNA analysis.

### Reagents:

rhuTNFa: R&D Systems, cat#210-TA-050, 50 ug/ml in PBS
rhuIL-17A: R&D Systems, cat# cat#317-IL/CF, 50 ug/ml in PBS
rhuIL-17C: Amgen lot 102641-82, P60765.31, 1.1 mg/ml, MW-20.9KD, C-tag: 6×HIS, expressed in 293-6E, material has 70% monomer and 30% dimer
Anti-huIL-17C monoclonal antibody, R&D Systems, cat#MAB 177, lot#GBZ03
IL-17RE-FC (176-451): Amgen lot# P61681.17, 4.6 mg/ml, MW 122KD, Endotoxin, 0.16 EU/mg
Recombinant human IL-17RA-Fc: R&D Systems, cat#177-IR,
NHEK cells: cat#00192627 (NHEK-Ad in KGM-Gold), lot#9F3069, from Lonza
Culture medium: KBM-Gold, cat#00192151 with KGM-Gold SingleQuot kit cat#00192152 (supplements and growth Factors), lot#0000179118
TNS: Trypsin Neutralizing Solution, cat# cc-5002, lot#0000134410, from Lonza
TrypsiwEDTA: cat# cc-5012, lot#0000121055, from Lonza
High-Capacity cDNA Reverse Transcription kits: AB Applied Biosystems (Foster City, Calif.), Cat#4368814
Human DEFB4 Taqman primer: Cat# HSOO175474, from AB Applied Biosystems
Human HPRT Taqman primer: Cat# Hs99999909, from AB Applied Biosystems
NHEK cells were stimulated with IL-17C or IL-17C+TNF-alpha for 48 hrs and harvested for RNA.
DEBF4 expression was measured via Taqman™. TLDA and microarray data showed that IL-17C induced expression of multiple genes from NHEK cells (data not shown).

### Assay Format:

### Medium

TNF-α 0.1 ug/ml
IL-17C 0.5 ug/ml
IL-17C 0.5 ug/ml+TNFa
IL-17C 0.1 ug/ml+TNFa
IL-17C 0.02 ug/ml+TNFa
IL-17C 0.004 ug/ml+TNFa
IL-17C 0.0008 ug/ml+TNFa
IL-17C (0.1 ug/ml)+TNFa+MAB 177 50 ug/ml
IL-17C (0.1 ug/ml)+TNFa+MAB177 12.5 ug/ml
IL-17C (0.1 ug/ml)+TNFa+MAB177 4.125 ug/ml
IL-17C (0.1 ug/ml)+TNFa+MAB 177 1.04 ug/ml
IL-17C (0.1 ug/ml)+TNF a+MAB177 0 0.25 ug/ml
IL-17C (0.1 ug/ml)+TNFa+IL-17RE-Fc 50 ug/ml
IL-17C (0.1 ug/ml)+TNFa+IL-17RE-Fc 12.5 ug/ml
IL-17C (0.1 ug/ml)+TNFa+IL-17RE-Fc 4.125 ug/ml
IL-17C (0.1 ug/ml)+TNFa+IL-17RA-Fc 50 ug/ml
IL-17C (0.1 ug/ml)+TNFa+IL-17RA-Fc 12.5 ug/ml
IL-17RA-Fc 50 ug/ml
IL-17RE-Fc 50 ug/ml
MAB 177 50 ug/ml

RNA purification and Taqman™ analysis: 0.5 ml RLT buffer from RNeasy™ Mini kit (Qiagen) was added to each well, and plates were placed on ice during processing. Cell lysates were transferred to a new tube for RNA purification using the RNeasy™ Mini kit and following the manufacturer's protocol. 2 μg of total RNA was used for cDNA synthesis using High-Capacity cDNA Reverse Transcription Kits (Applied Biosystems, Foster City, Calif.), according to the manufacturer's instructions. cDNA samples were analyzed for expression of DEFB4 by Taqman. Taqman was performed using an Applied Biosystems 9600 Real Time PCR System. Results were normalized to the expression of HPRT and graphs were generated using Prism v5.

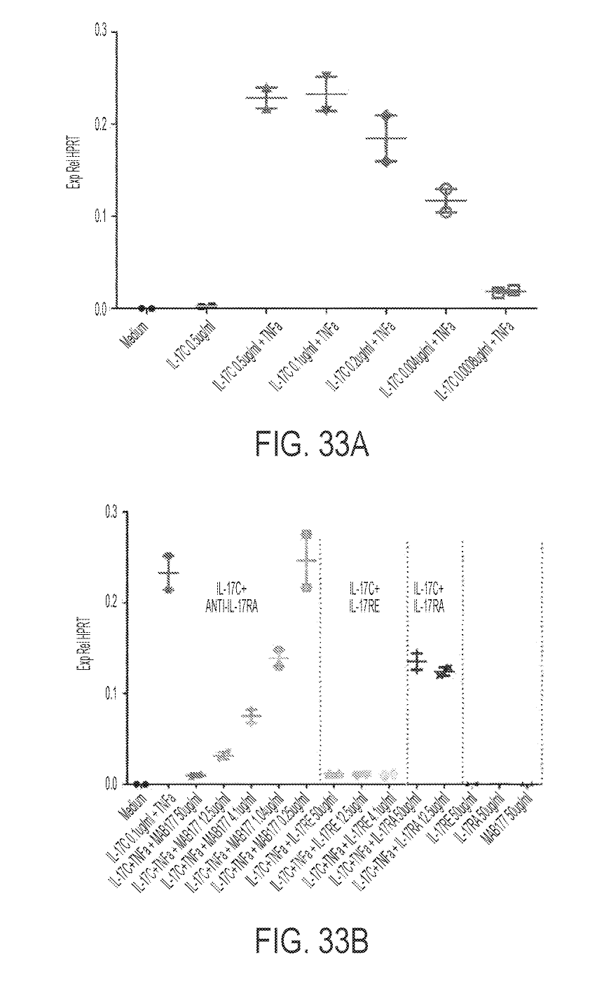

As shown in FIG. 33A, IL-17C in the presence of TNF-alpha induced DEFB4 gene expression in NHEK cells in a dose dependent manner. FIG. 33B shows that IL-17C biological activity (as measured by D EFB4 gene expression) is inhibited by IL-17RE-F c, IL-17RA-Fc and an anti-IL-17RA monoclonal antibody.

### 7. Human Antibodies to Human IL-17RA Inhibit the Biological Activity of IL-17C

The experiments described herein demonstrate that IL-17RA is necessary for IL-17C-induced responses, and that inhibition of IL-17C in addition to IL-17A could be key to the increased efficacy seen with IL-17RA inhibition compared with IL-17A inhibition (as shown in the mouse skin inflammation (psoriasis-like) model).

Human mAbs to human IL-17RA that are known to neutralize IL-17A, IL-17F, IL-17A/F, and IL-25 can be used to inhibit IL-17C. A specific embodiment, the human mAb AM14 (also referred to herein as AMG 827) was tested for its ability to inhibit the biological activity of IL-17C.

### Reagents:

rhuTNFa: R&D Systems, cat#210-TA-050, 50 ug/ml in PBS
rhuIL-17A: R&D Systems, cat# cat#317-IL/CF, 50 ug/ml in PBS
rhuIL-17C: Amgen lot 102641-82, P60765.31, 1.1 mg/ml, MW-20.9KD, C-tag: 6×HIS, expressed in 293-6E, material has 70% monomer and 30% dimer
Anti-huIL-17RA monoclonal antibody, R&D Systems, cat#MAB177, lot#GBZ03
Human anti-huIL-17RA monoclonal antibody AM14 (also referred to herein as AMG 827)
IL-17RE-FC (176-451): Amgen, lot# P61681.17, 4.6 mg/ml, MW 122KD, Endotoxin 0.16 EU/mg
Recombinant human IL-17RA-Fc: R&D Systems, cat#177-IR
NHEK cells: cat#00192627 (NHEK-Ad in KGM-Gold), lot#9F3069, from Lonza
Culture medium: KBM-Gold, cat#00192151 with KGM-Gold SingleQuot kit cat#00192152 (supplements and growth Factors), lot#0000179118
TNS: Trypsin Neutralizing Solution, cat# cc-5002, lot#0000134410, from Lonza
Trypsin/EDTA: cat# cc-5012, lot#0000121055, from Lonza
High-Capacity cDNA Reverse Transcription kits: AB Applied Biosystems, Cat#4368814
Human DEFB4 Taqman™ primer: Cat# HS00175474, from AB Applied Biosystems
Human HPRT Taqman™ primer: Cat# Hs99999909, from AB Applied Biosystems

### 1. Medium

2. TNF-α 0.1 ug/ml (R&D)
3. IL-17A 0.1 ug/ml (R&D)
4. IL-17A 0.1 ug/ml+TNFa
5. IL-17A 0.02 ug/ml+TNFa
6. IL-17A 0.004 ug/ml+TNFa
7. IL-17A 0.0008 ug/ml+TNFa
8. IL-17A 0.00016 ug/ml+TNFa
9. IL-17A 0.000032 ug/ml+TNFa
10. IL-17A 0.1 ug/ml+TNF a+MAB 177 50 ug/ml
11. IL-17A 0.1 ug/ml+TNFa+MAB177 12.5 ug/ml
12. IL-17A 0.1 ug/ml+TNFa+MAB 177 4.125 ug/ml
13. IL-17A 0.1 ug/ml+TNFa+MAB177 1.04 ug/ml
14. IL-17A 0.1 ug/ml+TNFa+MAB 177 0.25 ug/ml
15. IL-17A 0.1 ug/ml+TNF a+MAB177 0.06 ug/ml
16. IL-17A 0.1 ug/ml+TNFa+AMG827 150 ug/ml
17. IL-17A 0.1 ug/ml+TNFa+AMG827 37.5 ug/ml
18. IL-17A 0.1 ug/ml+TNFa+MAB177 9.3 ug/ml
19. IL-17A 0.1 ug/ml+TNFa+MAB177 2.3 ug/ml
20. IL-17A 0.1 ug/ml+TNFa+MAB177 0.58 ug/ml
21. IL-17A 0.1 ug/ml+TNFa+MAB 177 0.145 ug/ml
22. IL-17C 0.5 ug/ml (Amgen)
23. IL-17C 0.5 ug/ml+TNFa
24. IL-17C 0.1 ug/ml+TNFa
25. IL-17C 0.02 ug/ml+TNFa
26. IL-17C 0.004 ug/ml+TNFa
27. IL-17C 0.0008 ug/ml+TNFa
28. IL-17C 0.00016 ug/ml+TNFa
29. IL-17C (0. L ug/ml)+TNFa+MAB 177 50 ug/ml
30. IL-17C (0.1 ug/ml)+TNFa+MAB 177 12.5 ug/ml
31. IL-17C (0.1 ug/ml)+TNFa+MAB 177 4.125 ug/ml
32. IL-17C (0.1 ug/ml)+TNFa+MAB17 1.04 ug/ml
33. IL-17C (0.1 ug/ml)+TNFa+MAB177 0.25 ug/ml
34. IL-17C (0.1 ug/ml)+TNFa+MAB 177 0.06 ug/ml
35. IL-17C (0.1 ug/ml)+TNFa+AM14 150 ug/ml
36. IL-17C (0.1 ug/ml)+TNFa+AM14 37.5 ug/ml
37. IL-17C (0.1 ug/ml)+TNFa+AM14 9.3 ug/ml
38. IL-17C (0.1 ug/ml)+TNFa+AM14 2.3 ug/ml
39. IL-17C (0.1 ug/ml)+TNFa+AM14 0.58 ug/ml
40. IL-17C (0.1 ug/ml)+TNFa+AM14 0.15 ug/ml
41. IL-17C (0.1 ug/ml)+TNFa+IL-17RE-Fc 10 ug/ml
42. IL-17C (0.1 ug/ml)+TNFa+IL-17RA-Fc 50 ug/ml (R&D)
43. IL-17C (0.02 ug/ml)+TNFa+IL-17RA-FC 50 ug/ml (R&D)
44. MAB 177 50 ug/ml
45. AM14 150 ug/ml

NHEK cell culture: Normal human epidermal keratinocytes (Lonza, Portsmouth, N.H.) were cultured in KGM medium with supplements (KGM-Gold Bullet kit, manufacturer) following the manufacture's protocol. At passage 3-4, 1×10⁵/ml cells were plated into 6 well tissue culture plates, 2 ml per well. After 24 hrs in culture, medium was removed and replaced with new medium with/without cytokines. Cells were cultured for 48 hrs and then harvested for RNA analysis.

RNA purification and Taqman™ analysis: 0.5 ml RL T buffer from RNeasy Mini kit (Qiagen) was added to each well and plates were placed on ice during processing. Cell lysates were transferred to a new tube for RNA purification using the RNeasy kit following the manufacturer's protocol. 2 μg of total RNA was used for cDNA synthesis using High-Capacity cDNA Reverse Transcription Kits (Applied Biosystems, Foster City, Calif.) according to the manufacturer's instructions. cDNA samples were analyzed for expression of DEFB4 by Taqman™. Taqman™ was performed using an Applied Biosystems 9600 Real Time PCR System. Results were normalized to the expression of HPRT and graphs were generated using Prism v5.

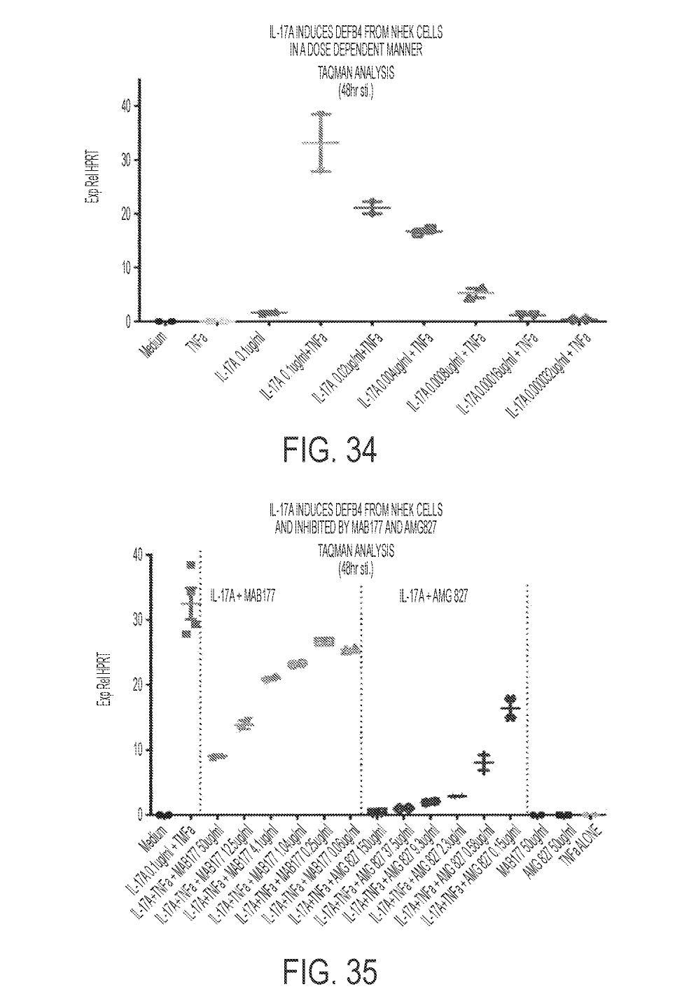

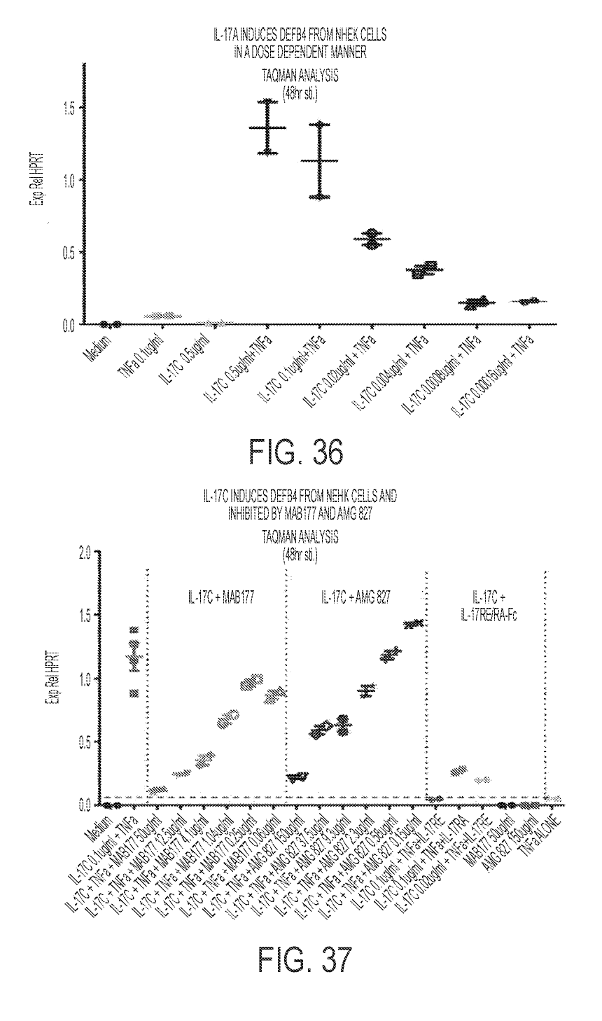

FIG. 34 shows that IL-17A induced DEFB4 from NHEK cells in a dose dependent manner. FIG. 35 shows that the biological activity of IL-17A on NHEK cells, as determined by DFEB4 expression, was inhibited by antibodies against IL-17RA, and in particular the monoclonal antibody AM14 (also referred to herein as AMG 827). FIG. 36 shows that IL-17C DEFB4 from NHEK cells in a dose dependent manner. FIG. 37 shows that the biological activity of IL-17C on NHEK cells, as determined by DFEB4 expression, was inhibited by antibodies against IL-17RA, and in particular the monoclonal antibody AM14 (also referred to herein as AMG 827).

This data provides evidence that select antibodies that bind IL-17RA have the ability to inhibit the biological activity of IL-17C.

Additional embodiments of human antibodies that specifically bind human IL-17RA and potentially inhibit IL-17C biological activity include AM12, AM16, AM17, AM19 and AM22, as well as antibodies, as variously defined herein, comprising the respective CDRs of these antibodies, as well as antibodies, as variously defined herein, comprising the respective variable heavy and/or light domains. These antibodies are IL-17RA-IL-17RE antagonists. Further embodiments of antibodies that may be used to inhibit the activity of IL-17C include the following:

| | | |
|---|---|---|
| Amino acid | SEQ ID NO: 1 | QVQLVQSGAEVKKPGASVKVSCKASGYTLT |
| sequence | | SYGISWVRQAPGQGLEWMGWISTYKGNTNY |
| AMH12Vh | | AQKLQGRVTMTTDTSTSTAYMELRSLRSDD |
| | | TAVYYCARKQLVFDYWGQGTLVTVSS |
| Amino acid | SEQ ID NO: 2 | EIVMTQSPATLSVSPGERATLSCRASQSISSSL |
| sequence | | AWYQQKPGQAPRLLIYGASTRATGIPARFSG |
| AML12 Vl | | SGSGTEFTLTISSLQSENFAVYYCQQYDNWP |
| | | LTFGGGTKVEIK |
| Amino acid | SEQ ID NO: 3 | QVQLVQSGAEVKKPGASVKVSCKASGYTFT |
| sequence | | RYGISWVRQAPGQGLEWMGWISTYSGNTNY |
| AM_{H}14 Vh | | AQKLQGRVTMTTDTSTSTAYMELRSLRSDD |
| | | TAVYYCARRQLYFDYWGQGTLVTVSS |
| Amino acid | SEQ ID NO: 4 | EIVMTQSPATLSVSPGERATLSCRASQSVSSN |
| sequence | | LAWFQQKPGQAPRPLIYDASTRATGVPARFS |
| AM_{L}14 Vl | | GSGSGTDFTLTISSLQSEDFAVYYCQQYDNW |
| | | PLTFGGGTKVEIK |
| Amino acid | SEQ ID NO: 5 | QVQLVQSGAEVKKPGASVKVSCKASGYTFT |
| sequence | | SYGISWVRQAPGQGLEWMGWISAYNGNTK |
| AMH16 Vh | | YAQKLQGRVTMTTDTSTSTVYMELRSLRSD |
| | | DTAVYYCARKQLVFDYWGQGTLVTVSS |
| Amino acid | SEQ ID NO: 6 | EIVMTQSPATLSVSPGERATLSCRASQSISTSL |
| sequence | | AWYQQKPGQAPRLLIYGTSTRATGIPARARFSG |
| AML16 Vl | | SGSGTEFTLTISSLQSEDFAVYFCQQYDIWPL |
| | | TFGGGTKVEIK |
| Amino acid | SEQ ID NO: 7 | QVQLVQSGAEVKKPGAAVKVSCKATGYTLT |
| sequence | | SYGISWVRQAPGQGLEWMGWISAYSGNTKY |
| AMH17Vh | | AQKLQGRVTMTTDTSTSTAYMELRSLRSDD |
| | | TAVYYCARKQLVFDYWGQGTLVTVSS |
| Amino acid | SEQ ID NO: 8 | EIVMTQSPATLSVSPGERATLSCRASQSVSSN |
| sequence | | LAWYQQKPGQAPRLLIYGASTRATGIPARFS |
| AML17Vl | | GSGSGTEFTLTISSLQSEDFAVYSCQQYDNW |
| | | PLTFGGGTKVEIK |
| Amino acid | SEQ ID NO: 9 | QVQLVQSGAEVKKPGASVKVSCKASGYTLT |
| sequence | | SYGISWVRQAPGQGLEWMGWISAYSGNTKY |
| AMH19Vh | | AQKFQGRVTMTTDTSTSTAYMELRSLRSDD |
| | | TAVYYCARRQLALDYWGQGTLVTVSS |
| Amino acid | SEQ ID NO: 10 | EIVMTQSPATLSVSPGERATLSCRASQSISSNL |
| sequence | | AWYQQKPGQAPRLLIYGASTRATGIPARFSD |
| AML19Vl | | NGSGTEFTLTISSLQSEDFAVYFCQQYDTWPL |
| | | TFGGGTKVEIK |
| Amino acid | SEQ ID NO: 11 | QVQLVQSGAEVKKPGASVKVSCKASGYTFT |
| sequence | | RYGISWVRQAPGQGLEWMGWISAYSGNTN |
| AMH22Vh | | YAQKLQGRVTMTTDTSTSTAYMELRSLRSD |
| | | DTAVYYCARRQLYFDYWGQGTLVTVSS |
| Amino acid | SEQ ID NO: 12 | EIVMTQSPATLSVSPGERVTLSCRASQSVSSN |
| sequence | | LAWFQQKPGQAPRPLIYDASTRAAGIPARFS |
| AML22Vl | | GSGSGTDFTLTISSLQSEDFAVYYCQQYDNW |
| | | PLTFGGGTKVEIK |
| Amino acid | SEQ ID NO: 13 | SYGIS |
| sequence of | | |
| CDR 1 of | | |
| AM_{H}12Vh | | |
| Amino acid | SEQ ID NO: 14 | WISTYKGNTNYAQKLQG |
| sequence of | | |
| CDR 2 of | | |
| AM_{H}12Vh | | |
| Amino acid | SEQ ID NO: 15 | KQLVFDY |
| sequence of | | |
| CDR 3 of | | |
| AM_{H}12Vh | | |
| Amino acid | SEQ ID NO: 16 | RYGIS |
| sequence of | | |
| CDR 1 of | | |
| AM_{H}14 Vh | | |
| Amino acid | SEQ ID NO: 17 | WISTYSGNTNYAQKLQG |
| sequence of | | |
| CDR 2of | | |
| AM_{H}14 Vh | | |
| Amino acid | SEQ ID NO: 18 | RQLYFDY |
| sequence of | | |
| CDR 3 of | | |
| AM_{H}14 Vh | | |
| Amino acid | SEQ ID NO: 19 | SYGIS |
| sequence of | | |
| CDR 1 of | | |
| AM_{H}16Vh | | |
| Amino acid | SEQ ID NO: 20 | WISAYNGNTKYAQKLQG |
| sequence of | | |
| CDR 2 of | | |
| AM_{H}16 Vh | | |
| Amino acid | SEQ ID NO: 21 | KQLVFDY |
| sequence of | | |
| CDR 3 of | | |
| AM_{H}16Vh | | |
| Amino acid | SEQ ID NO: 22 | SYGIS |
| sequence of | | |
| CDR 1 of | | |
| AM_{H}17Vh | | |
| Amino acid | SEQ ID NO: 23 | WISAYSGNTKYAQKLQG |
| sequence of | | |
| CDR 2of | | |
| AM_{H}17V | | |
| Amino acid | SEQ ID NO: 24 | KQLVFDY |
| sequence of | | |
| CDR3 of | | |
| AM_{H}17Vh | | |
| Amino acid | SEQ ID NO: 25 | SYGIS |
| sequence of | | |
| CDR 1 of | | |
| AM_{H}19 Vh | | |
| Amino acid | SEQ ID NO: 26 | WISAYSGNTKYAQKLQG |
| sequence of | | |
| CDR 2of | | |
| AM_{H}19Vh | | |
| Amino acid | SEQ ID NO: 27 | RQLALDY |
| sequence of | | |
| CDR3 of | | |
| AM_{H}19 Vh | | |
| Amino acid | SEQ ID NO: 28 | WISAYSGNTNYAQKLQG |
| sequence of | | |
| CDR 2 of | | |
| AM_{H}22Vh | | |
| Amino acid | SEQ ID NO: 29 | RQLYFDY |
| sequence of | | |
| CDR 3 of | | |
| AM_{H}22Vh | | |
| Amino acid | SEQ ID NO: 30 | RASQSISSSLA |
| sequence of | | |
| CDR 1 of | | |
| AM_{L}12Vl | | |
| Amino acid | SEQ ID NO: 31 | GASTRAT |
| sequence of | | |
| CDR 2 of | | |
| AM_{L}12 Vl | | |
| Amino acid | SEQ ID NO: 32 | QQYDNWPLT |
| sequence of | | |
| CDR 3 of | | |
| AM_{L}12 V1 | | |
| Amino acid | SEQ ID NO: 33 | RASQSVSSNLA |
| sequence of | | |
| CDR 1 of | | |
| AM_{L}14 Vl | | |
| Amino acid | SEQ ID NO: 34 | DASTRAT |
| sequence of | | |
| CDR 2of | | |
| AM_{L}14 Vl | | |
| Amino acid | SEQ ID NO: 35 | QQYDNWPLT |
| sequence of | | |
| CDR 3 of | | |
| AM_{L}14 Vl | | |
| Amino acid | SEQ ID NO: 36 | RASQSISTSLA |
| sequence of | | |
| CDR 1 of | | |
| AM_{L}16Vl | | |
| Amino acid | SEQ ID NO: 37 | GTSTRAT |
| sequence of | | |
| CDR 2 of | | |
| AM_{L}16 Vl | | |
| Amino acid | SEQ ID NO: 38 | QQYDIWPLT |
| sequence of | | |
| CDR 3 of | | |
| AM_{L}16 Vl | | |
| Amino acid | SEQ ID NO: 39 | RASQSVSSNLA |
| sequence of | | |
| CDR 1 of | | |
| AM_{L}17Vl | | |
| Amino acid | SEQ ID NO: 40 | GASTRAT |
| sequence of | | |
| CDR2of | | |
| AM_{L}17Vl | | |
| Amino acid | SEQ ID NO: 41 | QQYDNWPLT |
| sequence of | | |
| CDR 3 of | | |
| AM_{L}17Vl | | |
| Amino acid | SEQ ID NO: 42 | RASQSISSNLA |
| sequence of | | |
| CDR 1 of | | |
| AM_{L}19Vl | | |
| Amino acid | SEQ ID NO: 43 | GASTRAT |
| sequence of | | |
| CDR 2 of | | |
| AM_{L}19Vl | | |
| Amino acid | SEQ ID NO: 44 | QQYDTWPLT |
| sequence of | | |
| CDR 3 of | | |
| AM_{L}19Vl | | |
| Amino acid | SEQ ID NO: 45 | RASQSVSSNLA |
| sequence of | | |
| CDR 1 of | | |
| AM_{L}22Vl | | |
| Amino acid | SEQ ID NO: 46 | DASTRAA |
| sequence of | | |
| CDR2 of | | |
| AM_{L}22Vl | | |
| Amino acid | SEQ ID NO: 47 | QQYDNWPLT |
| sequence of | | |
| CDR 3 of | | |
| AM_{L}22 Vl | | |
| Amino acid | SEQ ID NO: 48 | MEWTWRVLFLVAAATGAHSQVQLVQSGAE |
| sequence | | VKKPGASVKVSCKASGYTFTRYGISWVRQA |
| AM_{H}14 | | PGQGLEWMGWISTYSGNTNYAQKLQGRVT |
| full- | | MTTDTSTSTAYMELRSLRSDDTAVYYCARR |
| length | | QLYFDYWGQGTLVTVSSASTKGPSVFPLAPC |
| heavy | | SRSTSESTAALGCLVKDYFPEPVTVSWNSGA |
| chain | | LTSGVHTFPAVLQSSGLYSLSSVVTVPSSNFG |
| | | TQTYTCNVDHKPSNTKVDKTVERKCCVECP |
| | | PCPAPPVAGPSVFLFPPKPKDTLMISRTPEVT |
| | | CVVVDVSHEDPEVQFNWYVDGVEVHNAKT |
| | | KPREEQFNSTFRVVSVLTVVHQDWLNGKEY |
| | | KCKVSNKGLPAPIEKTISKTKGQPREPQVYTL |
| | | PPSREEMTKNQVSLTCLVKGFYPSDIAVEWE |
| | | SNGQPENNYKTTPPMLDSDGSFFLYSKLTVD |
| | | KSRWQQGNVFSCSVMHEALHNHYTQKSLSL |
| | | SPGK |
| Amino acid | SEQ ID NO: 49 | EIVMTQSPATLSVSPGERATLSCRASQSVSSN |
| sequence | | LAWFQQKPGQAPRPLIYDASTRATGVPARFS |
| AM_{L}14 | | GSGSGTDFTLTISSLQSEDFAVYYCQQYDNW |
| full- | | PLTFGGGTKVEIKRTVAAPSVFIFPPSDEQLKS |
| length | | GTASVVCLLNNFYPREAKVQWKVDNALQSG |
| light | | NSQESVTEQDSKDSTYSLSSTLTLSKADYEK |
| chain | | HKVYACEVTHQGLSSPVTKSFNRGEC |

What is claimed is:

1.-16. (canceled)

17. A method of inhibiting release of at least one pathogenic mediator from a cell expressing an IL-17RA-IL-17RE heteromeric receptor complex, comprising exposing said cell expressing IL-17RA, IL-17RE, and IL-17RA-IL-17RE heteromeric receptor complex to an IL-17RA-IL-17RE antagonist such that IL-17C is partially or fully inhibited from activating said IL-17RA-IL-17RE heteromeric receptor complex.

18. The method of claim 17, wherein the IL-17RA-IL-17RE antagonist is an antibody.

19. The method of claim 18, wherein the antibody binds the IL-17RA-IL-17RE heteromeric receptor complex.

20. The method of claim 17, wherein the pathogenic mediator is selected from the group consisting of: IL-6, IL-8, G-CSF, GM-CSF, TNFα, lipoclin-2, DEFB4, S100a8, and S100a9.

21. A method of inhibiting IL-17RA-IL-17RE heteromeric receptor complex formation in vivo, comprising exposing a cell expressing IL-17RA, IL-17RE, and IL-17RA-IL-17RE heteromeric receptor complex to an IL-17RA-IL-17RE antagonist such that IL-17RA and IL-17RE are partially or fully inhibited from forming an IL-17RA-IL-17RE heteromeric receptor complex.

22. The method of claim 21, wherein the IL-17RA-IL-17RE antagonist is an antibody.

23. The method of claim 22, wherein the antibody binds the IL-17RA-IL-17RE heteromeric receptor complex.

24. A method of inhibiting IL-17RA-IL-17RE heteromeric receptor complex activation in vivo, comprising exposing a cell expressing IL-17RA, IL-17RC, and IL-17RA-IL-17RE heteromeric receptor complex to an IL-17RA-IL-17RE antagonist such that IL-17C is partially or fully inhibited from activating said IL-17RA-IL-17RE heteromeric receptor complex.

25. The method of claim 24, wherein the IL-17RA-IL-17RE antagonist is an antibody.

26. The method of claim 25, wherein the antibody binds the IL-17RA-IL-17RE heteromeric receptor complex.

27. A method of inhibiting release of at least one pathogenic mediator in vivo from a cell expressing an IL-17RA-IL-17RE heteromeric receptor complex, comprising exposing said cell expressing IL-17RA, IL-17RE, and IL-17RA-IL-17RE heteromeric receptor complex to an IL-17RA-IL-17RE antagonist such that IL-17C is partially or fully inhibited from activating said IL-17RA-IL17RE heteromeric receptor complex.

28. The method of claim 27, wherein the IL-17RA-IL-17RE antagonist is an antibody.

29. The method of claim 28, wherein the antibody binds the IL-17RA-IL-17RE heteromeric receptor complex.

30. The method of claim 27, wherein the pathogenic mediator is selected from the group consisting of: IL-6, IL-8, G-CSF, GM-CSF, TNFα, lipoclin-2, DEFB4, S100a8, and S100a9.

31. A method of reducing at least one pathogenic mediator in a patient having an autoimmune disease, comprising administering an IL-17RA-IL-17RE antagonist to said patient, wherein said patient's cells express IL-17RA, IL-17RE, and IL-17RA-IL-17RE heteromeric receptor complex, and wherein said antagonist partially or fully inhibits IL-17C from activating said IL-17RA-IL-17RE heteromeric receptor complex.

32. The method of claim 31, wherein the IL-17RA-IL-17RE antagonist is an antibody.

33. The method of claim 34, wherein the antibody binds the IL-17RA-IL-17RE heteromeric receptor complex.

34. The method of claim 31, wherein the pathogenic mediator is selected from the group consisting of: IL-6, IL-8, G-CSF, GM-CSF, TNFα, lipoclin-2, DEFB4, S100a8, and S100a9.

35. A method of reducing at least one pathogenic mediator in a patient having an inflammatory disease, comprising administering an IL-17RA-IL-17RE antagonist to said patient, wherein said patient's cells express IL-17RA, IL-17RE, and IL-17RA-IL-17RE heteromeric receptor complex, and wherein said antagonist partially or fully inhibits IL-17A and/or IL-17F from activating said IL-17RA-IL-17RE heteromeric receptor complex.

36. The method of claim 35, wherein the IL-17RA-IL-17RE antagonist is an antibody.

37. The method of claim 36, wherein the antibody binds the IL-17RA-IL-17RE heteromeric receptor complex.

38. The method of claim 35, wherein the pathogenic mediator is selected from the group consisting of: IL-6, IL-8, G-CSF, GM-CSF, TNFα, lipoclin-2, DEFB4, S100a8, and S100a9.

39. A method of reducing at least one pathogenic mediator in a patient having rheumatoid arthritis, comprising administering an IL-17RA-IL-17RE antagonist to said patient, wherein said patient's cells express IL-17RA, IL-17RE, and IL-17RA-IL-17RE heteromeric receptor complex, and wherein said antagonist partially or fully inhibits IL-17C from activating said IL-17RA-IL-17RE heteromeric receptor complex.

40. The method of claim 39, wherein the IL-17RA-IL-17RE antagonist is an antibody.

41. The method of claim 40, wherein the antibody binds the IL-17RA-IL-17RE heteromeric receptor complex.

42. The method of claim 39, wherein the pathogenic mediator is selected from the group consisting of: IL-6, IL-8, G-CSF, GM-CSF, TNFα, lipoclin-2, DEFB4, S100a8, and S100a9.

43. A method of reducing at least one pathogenic mediator in a patient having inflammatory bowel disease, comprising administering an IL-17RA-IL-17RE antagonist to said patient, wherein said patient's cells express IL-17RA, IL-17RE, and IL-17RA-IL-17RE heteromeric receptor complex, and wherein said antagonist partially or fully inhibits IL-17C from activating said IL-17RA-IL-17RE heteromeric receptor complex.

44. The method of claim 43, wherein the IL-17RA-IL-17RE antagonist is an antibody.

45. The method of claim 44, wherein the antibody binds the IL-17RA-IL-17RE heteromeric receptor complex.

46. The method of claim 43, wherein the pathogenic mediator is selected from the group consisting of: IL-6, IL-8, G-CSF, GM-CSF, TNFα, lipoclin-2, DEFB4, S100a8, and S100a9.

47. A method of reducing at least one pathogenic mediator in a patient having psoriasis, comprising administering an IL-17RA-IL-17RE antagonist to said patient, wherein said patient's cells express IL-17RA, IL-17RE, and IL-17RA-IL-17RE heteromeric receptor complex, and wherein said antagonist partially or fully inhibits IL-17C from activating said IL-17RA-IL-17RE heteromeric receptor complex.

48. The method of claim 47, wherein the IL-17RA-IL-17RE antagonist is an antibody.

49. The method of claim 48, wherein the antibody binds the IL-17RA-IL-17RE heteromeric receptor complex.

50. The method of claim 47, wherein the pathogenic mediator is selected from the group consisting of: IL-6, IL-8, G-CSF, GM-CSF, TNFα, lipoclin-2, DEFB4, S100a8, and S100a9.

51. A method of reducing at least one pathogenic mediator in a patient having asthma, comprising administering an IL-17RA-IL-17RE antagonist to said patient, wherein said patient's cells express IL17RA, IL-17RE, and IL-17RA-IL-17RE heteromeric receptor complex, and wherein said antagonist partially or fully inhibits IL-17A and/or IL-17F from activating said IL-17RA-IL-17RE heteromeric receptor complex.

52. The method of claim 51, wherein the IL-17RA-IL-17RE antagonist is an antibody.

53. The method of claim 52, wherein the antibody binds the IL-17RA-IL-17RE heteromeric receptor complex.

54. The method of claim 51, wherein the pathogenic mediator is selected from the group consisting of: IL-6, IL-8, G-CSF, GM-CSF, TNFα, lipoclin-2, DEFB4, S100a8, and S100a9.

55. A method of reducing at least one pathogenic mediator in a patient having psoriasis, comprising administering an IL-17RA-IL-17RE antagonist to said patient, wherein said patient's cells express IL-17RA, IL-17RE, and IL-17RA-IL-17RE heteromeric receptor complex, and wherein said antagonist partially or fully inhibits IL-17C from activating said IL-17RA-IL-17RE heteromeric receptor complex.

56. The method of claim 55, wherein the IL-17RA-IL-17RE antagonist is an antibody.

57. The method of claim 56, wherein the antibody binds the IL-17RA-IL-17RE heteromeric receptor complex.

58. The method of claim 55, wherein the pathogenic mediator is selected from the group consisting of: IL-6, IL-8, G-CSF, GM-CSF, TNFα, lipoclin-2, DEFB4, S100a8, and S100a9.

59.-64. (canceled)


SEQUENCE LISTING (61 sequences)

SEQ ID NO: 1 - protein, 116 aa, Homo sapiens
QVQLVQSGAEVKKPGASVKVSCKASGYTLTSYGISWVRQAPGQGLEWMGWISTYKGNTNYAQKLQGRVTMTTDTSTSTAYMELRSLRSDDTAVYYCARKQLVFDYWGQGTLVTVSS

SEQ ID NO: 2 - protein, 107 aa, Homo sapiens
EIVMTQSPATLSVSPGERATLSCRASQSISSSLAWYQQKPGQAPRLLIYGASTRATGIPARFSGSGSGTEFTLTISSLQSENFAVYYCQQYDNWPLTFGGGTKVEIK

SEQ ID NO: 3 - protein, 116 aa, Homo sapiens
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 4 - protein, 107 aa, Homo sapiens
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

SEQ ID NO: 5 - protein, 116 aa, Homo sapiens
QVQLVQSGAEVKKPGASVKVSCKASGYTFTSYGISWVRQAPGQGLEWMGWISAYNGNTKYAQKLQGRVTMTTDTSTSTVYMELRSLRSDDTAVYYCARKQLVFDYWGQGTLVTVSS

SEQ ID NO: 6 - protein, 107 aa, Homo sapiens
EIVMTQSPATLSVSPGERATLSCRASQSISTSLAWYQQKPGQAPRLLIYGTSTRATGIPARFSGSGSGTEFTLTISSLQSEDFAVYFCQQYDIWPLTFGGGTKVEIK

SEQ ID NO: 7 - protein, 116 aa, Homo sapiens
QVQLVQSGAEVKKPGAAVKVSCKATGYTLTSYGISWVRQAPGQGLEWMGWISAYSGNTKYAQKLQGRVTMTTDTSTSTAYMELRSLRSDDTAVYYCARKQLVFDYWGQGTLVTVSS

SEQ ID NO: 8 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYSCQQYDNWPLTFGGGTKVEIK

SEQ ID NO: 9 - protein, 116 aa, Homo sapiens
QVQLVQSGAEVKKPGASVKVSCKASGYTLTSYGISWVRQAPGQGLEWMGWISAYSGNTKYAQKFQGRVTMTTDTSTSTAYMELRSLRSDDTAVYYCARRQLALDYWGQGTLVTVSS

SEQ ID NO: 10 - protein, 107 aa, Homo sapiens
EIVMTQSPATLSVSPGERATLSCRASQSISSNLAWYQQKPGQAPRLLIYGASTRATGIPARFSDNGSGTEFTLTISSLQSEDFAVYFCQQYDTWPLTFGGGTKVEIK

SEQ ID NO: 11 - protein, 116 aa, Homo sapiens
QVQLVQSGAEVKKPGASVKVSCKASGYTFTRYGISWVRQAPGQGLEWMGWISAYSGNTNYAQKLQGRVTMTTDTSTSTAYMELRSLRSDDTAVYYCARRQLYFDYWGQGTLVTVSS

SEQ ID NO: 12 - protein, 107 aa, Homo sapiens
EIVMTQSPATLSVSPGERVTLSCRASQSVSSNLAWFQQKPGQAPRPLIYDASTRAAGIPARFSGSGSGTDFTLTISSLQSEDFAVYYCQQYDNWPLTFGGGTKVEIK

SEQ ID NO: 13 - protein, 5 aa, Homo sapiens
SYGIS

SEQ ID NO: 14 - protein, 17 aa, Homo sapiens
WISTYKGNTNYAQKLQG

SEQ ID NO: 15 - protein, 7 aa, Homo sapiens
KQLVFDY

SEQ ID NO: 16 - protein, 5 aa, Homo sapiens
RYGIS

SEQ ID NO: 17 - protein, 17 aa, Homo sapiens
WISTYSGNTNYAQKLQG

SEQ ID NO: 18 - protein, 7 aa, Homo sapiens
RQLYFDY

SEQ ID NO: 19 - protein, 5 aa, Homo sapiens
SYGIS

SEQ ID NO: 20 - protein, 17 aa, Homo sapiens
WISAYNGNTKYAQKLQG

SEQ ID NO: 21 - protein, 7 aa, Homo sapiens
KQLVFDY

SEQ ID NO: 22 - protein, 5 aa, Homo sapiens
SYGIS

SEQ ID NO: 23 - protein, 17 aa, Homo sapiens
WISAYSGNTKYAQKLQG

SEQ ID NO: 24 - protein, 7 aa, Homo sapiens
KQLVFDY

SEQ ID NO: 25 - protein, 5 aa, Homo sapiens
SYGIS

SEQ ID NO: 26 - protein, 17 aa, Homo sapiens
WISAYSGNTKYAQKFQG

SEQ ID NO: 27 - protein, 7 aa, Homo sapiens
RQLALDY

SEQ ID NO: 28 - protein, 17 aa, Homo sapiens
WISAYSGNTNYAQKLQG

SEQ ID NO: 29 - protein, 7 aa, Homo sapiens
RQLYFDY

SEQ ID NO: 30 - protein, 11 aa, Homo sapiens
RASQSISSSLA

SEQ ID NO: 31 - protein, 7 aa, Homo sapiens
GASTRAT

SEQ ID NO: 32 - protein, 9 aa, Homo sapiens
QQYDNWPLT

SEQ ID NO: 33 - protein, 11 aa, Homo sapiens
RASQSVSSNLA

SEQ ID NO: 34 - protein, 7 aa, Homo sapiens
DASTRAT

SEQ ID NO: 35 - protein, 9 aa, Homo sapiens
QQYDNWPLT

SEQ ID NO: 36 - protein, 11 aa, Homo sapiens
RASQSISTSLA

SEQ ID NO: 37 - protein, 7 aa, Homo sapiens
GTSTRAT

SEQ ID NO: 38 - protein, 9 aa, Homo sapiens
QQYDIWPLT

SEQ ID NO: 39 - protein, 11 aa, Homo sapiens
RASQSVSSNLA

SEQ ID NO: 40 - protein, 7 aa, Homo sapiens
GASTRAT

SEQ ID NO: 41 - protein, 9 aa, Homo sapiens
QQYDNWPLT

SEQ ID NO: 42 - protein, 11 aa, Homo sapiens
RASQSISSNLA

SEQ ID NO: 43 - protein, 7 aa, Homo sapiens
GASTRAT

SEQ ID NO: 44 - protein, 9 aa, Homo sapiens
QQYDTWPLT

SEQ ID NO: 45 - protein, 11 aa, Homo sapiens
RASQSVSSNLA

SEQ ID NO: 46 - protein, 7 aa, Homo sapiens
DASTRAA

SEQ ID NO: 47 - protein, 9 aa, Homo sapiens
QQYDNWPLT

SEQ ID NO: 48 - protein, 461 aa, Homo sapiens
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 49 - protein, 214 aa, Homo sapiens
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - DNA, 33 bp, Artificial Sequence
  Synthetic Primer
agctagctaaccggtgcagcgatcatccctcaa

SEQ ID NO: 51 - DNA, 46 bp, Artificial Sequence
  Synthetic Primer
atgtgtgagttttgtcgcttccaccgcctccggacgcatggcggac

SEQ ID NO: 52 - DNA, 46 bp, Artificial Sequence
  Synthetic Primer
gtccgccatgcgtccggaggcggtggaagcgacaaaactcacacat

SEQ ID NO: 53 - DNA, 59 bp, Artificial Sequence
  Synthetic Primer
ctagctagctgcggccgcctatttatcatcatcatctttataatctttacccggagaca

SEQ ID NO: 54 - DNA, 33 bp, Artificial Sequence; identical to SEQ ID NO: 50
  Synthetic Primer

SEQ ID NO: 55 - DNA, 59 bp, Artificial Sequence; identical to SEQ ID NO: 53
  Synthetic Primer

SEQ ID NO: 56 - DNA, 21 bp, Artificial Sequence
  Synthetic Primer
agctagcaccggtgatccccc

SEQ ID NO: 57 - DNA, 46 bp, Artificial Sequence
  Synthetic Primer
atgtgtgagttttgtcgcttccaccgcctccctgtgtagacctggg

SEQ ID NO: 58 - DNA, 46 bp, Artificial Sequence
  Synthetic Primer
cccaggtctacacagggaggcggtggaagcgacaaaactcacacat

SEQ ID NO: 59 - DNA, 59 bp, Artificial Sequence; identical to SEQ ID NO: 53
  Synthetic Primer

SEQ ID NO: 60 - DNA, 21 bp, Artificial Sequence
  Synthetic Primer
agctagcaccggtgatccccc

SEQ ID NO: 61 - DNA, 59 bp, Artificial Sequence; identical to SEQ ID NO: 53
  Synthetic Primer